(19) United States
(12) Patent Application Publication
(10) Pub. No.: US 2009/0186028 A1
(43) Pub. Date: Jul. 23, 2009

(54) Detection of mutations in a gene associated with resistance to viral infection

(75) Inventors: Shawn P. Iadonato, Seattle, WA (US); Charles L. Magness, Seattle, WA (US); Christina A. Scherer, Seattle, WA (US)

Correspondence Address: C. Rachal Winger, 925 Fourth Avenue, Suite 2900, Seattle, WA, 98104, US

(73) Assignee: Illumigen Biosciences, Inc., Seattle, WA (US)

(21) Appl. No.: 11/630,195

(22) PCT Filed: Jun. 27, 2005
(86) PCT No.: PCT/US05/22729

§ 371 Date: Jun. 10, 2008

Related U.S. Application Data
(60) Provisional application No. 60/583,503, filed on Jun. 28, 2004.

(51) Int. Cl. A61K 39/395; C12Q 1/68; C12Q 1/70; A61P 31/12

(52) U.S. Cl. 424/139.1; 435/6; 435/5

(57) ABSTRACT

A method for detecting a mutation related to the gene encoding LDLR. This and other disclosed mutations correlate with resistance of humans to viral infection including hepatitis C. Also provided is a therapeutic agent consisting of a protein or polypeptide encoded by the wild-type and mutated genes, or a polynucleotide encoding the protein or polypeptide. Inhibitors of human LDLR, including antisense oligonucleotides, methods, and compositions specific for human LDLR, are also provided.

### 1. TECHNICAL FIELD

The present invention relates to a method for detecting a mutation in a human low density lipoprotein receptor gene, wherein a mutation confers resistance to viral infection, including flavivirus infection, and including infection by hepatitis C virus. The invention also relates to treating hepatitis C and other viral infections by mimicking naturally occurring virus resistance mutations discovered in the human population.

### 2. BACKGROUND OF THE INVENTION

The hepatitis C virus (HCV) is a flavivirus that is responsible for infection of more than 4 million persons in the United States and more than 170 million people worldwide. HCV infection is the leading cause of liver disease necessitating liver transplantation in the United States. Eighty-five percent or more of subjects infected with HCV genotype 1, the most common genotype in the United States, develop a chronic infection with associated progressive liver disease. The only approved treatment for HCV infection, a combination of interferon and ribavirin, results in viral clearance in fewer than 50% of treated subjects, many of whom experience intolerable side-effects during therapy. Clearly additional novel therapeutic strategies are needed to treat this disease.

Hepatitis C research and drug development have been significantly hampered by the lack of a cell culture and small animal model of viral infection. To date, no reliable cell culture system for propagating the virus has been developed, and the only species susceptible to HCV infection aside from human is the chimpanzee (Pan troglodytes). However, chimpanzees develop an atypical HCV infection with little or no liver disease and are generally difficult and expensive to husband for animal research. Because of these difficulties, a detailed analysis of the biochemistry of the host-virus interaction has yet to yield much in the way of definitive results.

A principal deficiency in our understanding of critical host-virus interactions involves the definitive identification of the cell surface receptor through which HCV initiates infection. For example, several groups have provided evidence for an association of the hepatitis C virus particle or the HCV envelop protein E2 with the cell surface tetraspanin CD81 (Cormier, E G, et al. Proc Natl Acad Sci USA. 101(19):7270-4, 2004; Zhang, J, et al. J Virol. 78(3):1448-55, 2004; Sasaki, M, et al. J Gastroenterol Hepatol. 18(1):74-9, 2003; Allander, T, et al., J Gen Virol. 81(10):2451-9, 2000; Petracca, R, et al. J Virol. 74(10):4824-30, 2000; Flint, M, et al. J Virol. 73(8):6782-90, 1999; Pileri, P, et al. Science. 282(5390):938-41, 1998). While many groups have confirmed the interaction between E2 and CD81, the preponderance of the evidence suggests that CD81 by itself is not sufficient to mediate viral entry into permissive cells (Bartosch, B, et al. J Biol Chem. 278(43):41624-30, 2003; Masciopinto, F, et al. Virology. 304(2):187-96, 2002; Flint, M, et al. Clin Liver Dis. 5(4):873-93, 2001; Meola, A, et al. J Virol. 74(13):5933-8, 2000). Other alternative receptors have been suggested to mediate HCV virus binding and entry, principally among these, the LDL receptor (LDLR) (Flint, M, et al. Clin Liver Dis. 5(4):873-93, 2001; Monazahian, M, et al. J Med Virol. 57(3):223-9, 1999; Agnello V, et al., Arthritis Rheum. 40(11):2007-15, 1997; Agnello, V. Springer Semin Immunopathol. 19(1):111-29, 1997). Several groups have demonstrated that HCV particles can bind to LDLR and be internalized by receptor mediated endocytosis (Agnello, V, et al., Proc Natl Acad Sci USA. 96(22):12766-71, 1999; Wunschmann, S, et al. J Virol. 74(21):10055-62, 2000; Germi, R, et al. J Med Virol. 68(2):206-15, 2002). Furthermore, HCV nucleocapsid particles have been shown to sediment in low density serum fractions along with lipoprotein particles such as LDL cholesterol (LDL-C) (Wunschmann, S, et al. J Virol. 74(21):10055-62, 2000). These data have led to speculation that HCV binding and viral entry into human cells is mediated by an interaction with LDL-C, apolipoprotein B, the LDLR, or some combination of the three. However, a definitive demonstration of LDLR as the principal cell surface receptor for viral entry has not been established.

### BRIEF SUMMARY OF THE INVENTION

The present invention describes definitive proof that LDLR is the functional cell surface receptor for the hepatitis C virus and is responsible for mediating HCV viral entry and infection. We further describe naturally occurring genetic mutations in the LDLR that confer host resistance to infection with HCV. We describe methods of treating HCV and other flavivirus infections by developing drugs that mimic the beneficial effects of these HCV resistance mutations. We further describe methods of treating HCV and other flavivirus infections by infusing LDL receptor subcomponents into human subjects. We finally describe methods for screening patient populations for identification of genetically conferred resistance to HCV and other viral infections.

The present invention relates to detecting hepatitis C resistance-related mutations which are characterized as point mutations in the low density lipoprotein receptor gene.

In one embodiment, a human genetic screening method is contemplated. The method comprises assaying a nucleic acid sample isolated from a human for the presence of a low density lipoprotein receptor gene mutation characterized as: a base substitution at nucleotide position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506056, or 2506062; a base insertion at 2506013; or a three-base deletion at 2506029-2506031, for low density lipoprotein receptor (LDLR) with reference to Genbank Sequence Accession No. NT_{—}011295.10 (consecutive nucleotides 2,460,001-2,509,020 of which are shown in SEQUENCE:1 and in FIG. 1).

In a preferred embodiment, the method comprises treating, under amplification conditions, a sample of genomic DNA from a human with a polymerase chain reaction (PCR) primer pair for amplifying a region of human genomic DNA containing nucleotide position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of low density liproprotein receptor gene NT_{—}011295.10. The PCR treatment produces an amplification product containing the region, which is then assayed for the presence of a point mutation. One preferred method of assaying the amplification product is DNA sequencing.

In a further embodiment, the invention provides a protein encoded by a gene having at least one mutation at position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of NT_{—}011295.10, and use of the protein to prepare a diagnostic for resistance to viral infection, preferably flaviviral infection, most preferably hepatitis C infection. In specific embodiments, the diagnostic is an antibody.

In a still further embodiment, the invention provides a therapeutic compound for preventing or inhibiting infection by a virus, preferably a flavivirus, most preferably the hepatitis C virus, wherein the therapeutic compound is a protein encoded by the LDLR gene.

In a still further embodiment, the invention provides a therapeutic compound for preventing or inhibiting infection by a virus, preferably a flavivirus, most preferably the hepatitis C virus, wherein the therapeutic compound is a protein encoded by an LDLR gene having at least one mutation at position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of NT_{—}011295.10. In other embodiments the therapeutic compound is a polynucleotide, such as DNA or RNA, encoding the protein.

In a still further embodiment, the invention provides a therapeutic compound for preventing or inhibiting infection by a virus, preferably a flavivirus, most preferably a hepatitis C virus, wherein the therapeutic compound is a protein of the sequence: SEQUENCE:10 or SEQUENCE:11.

In a still further embodiment, the invention provides a therapeutic compound for preventing or inhibiting infection by a virus, preferably a flavivirus, most preferably a hepatitis C virus, wherein the therapeutic compound is a protein comprised of at least 10, 15, 20 or more consecutive amino acids of the polypeptides of sequence: SEQUENCE:10 or SEQUENCE:11.

In a still further embodiment, the invention provides a therapeutic compound for preventing or inhibiting infection by a virus, preferably a flavivirus, most preferably hepatitis C virus, wherein the therapeutic compound mimics the beneficial effects of at least one mutation at position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of NT_{—}011295.10. The therapeutic compound can be a small molecule, protein, peptide, DNA or RNA molecule, or antibody.

In a still further embodiments, the therapeutic compound is capable of inhibiting the activity of LDLR or at least one sub-region or sub-function of the entire protein, and such compounds are represented by small molecules, antisense molecules, ribozymes, and RNAi molecules capable of specifically binding to LDLR polynucleotides, and by antibodies and fragments thereof capable of specifically binding to LDLR proteins and polypeptides, and by LDLR ligands and fragments thereof capable of specifically binding to LDLR proteins and polypeptides.

The present invention provides, in another embodiment, inhibitors of LDLR. Inventive inhibitors include, but are not limited to, antisense molecules, ribozymes, RNAi, antibodies or antibody fragments, proteins or polypeptides as well as small molecules. Exemplary antisense molecules comprise at least 10, 15 or 20 consecutive nucleotides of, or that hybridize under stringent conditions to the polynucleotide of SEQUENCE:1 or SEQUENCE:3. More preferred are antisense molecules that comprise at least 25 consecutive nucleotides of, or that hybridize under stringent conditions to the sequence of SEQUENCE:1 or SEQUENCE:3.

In a still further embodiment, inhibitors of LDLR are envisioned that are comprised of antisense or RNAi molecules that specifically bind or hybridize to the polynucleotide of SEQUENCE:2 or SEQUENCE:4.

In a still further embodiment, inhibitors of LDLR are envisioned that specifically bind to the region of the protein defined by the polypeptide of SEQUENCE:10. Inventive inhibitors include but are not limited to antibodies, antibody fragments, small molecules, proteins, or polypeptides.

In a still further embodiment, inhibitors of viral infection are envisioned that are derived from the natural ligands of LDLR. Inventive inhibitors include but are not limited to the polypeptides of SEQUENCE:12 or SEQUENCE:13 comprising the APOB and APOE proteins respectively. More preferred are polypeptides that comprise at least 10, 15, 20, or 25 consecutive amino acids of the polypeptides of SEQUENCE:12 or SEQUENCE:13.

In further embodiments, compositions are provided that comprise one or more LDLR inhibitors in a pharmaceutically acceptable carrier.

Additional embodiments provide methods of decreasing LDLR gene expression or biological activity.

Additional embodiments provide methods for decreasing the fraction of available LDLR protein at the cell surface. Preferred embodiments are compounds that stimulate LDLR endocytosis or retention of LDLR in the endosomal or lysosomal compartments.

Additional embodiments provide for methods of specifically increasing or decreasing the expression of certain forms of the LDLR gene having at least one mutation at position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of NT_{—}011295.10.

The invention provides an antisense oligonucleotide comprising at least one modified internucleoside linkage.

The invention further provides an antisense oligonucleotide having a phosphorothioate linkage.

The invention still further provides an antisense oligonucleotide comprising at least one modified sugar moiety.

The invention also provides an antisense oligonucleotide comprising at least one modified sugar moiety which is a 2′-O-methyl sugar moiety.

The invention further provides an antisense oligonucleotide comprising at least one modified nucleobase.

The invention still further provides an antisense oligonucleotide having a modified nucleobase wherein the modified nucleobase is 5-methylcytosine.

The invention also provides an antisense compound wherein the antisense compound is a chimeric oligonucleotide.

The invention provides a method of inhibiting the expression of human LDLR in human cells or tissues comprising contacting the cells or tissues in vivo with an antisense compound or a ribozyme of 8 to 35 nucleotides in length targeted to a nucleic acid molecule encoding human LDLR so that expression of human LDLR is inhibited.

The invention further provides a method of decreasing or increasing expression of specific forms of LDLR in vivo, such forms being defined by having at least one mutation at position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of NT_{—}011295.10, using antisense or RNAi compounds or ribozymes.

The invention further provides a method of increasing expression of specific forms of LDLR in vivo by delivering a gene therapy vector containing the LDLR gene having at least one mutation at position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of NT_{—}011295.10. Preferred embodiments include lentivirus, retrovirus, and adenovirus—derived gene therapy vectors.

The invention further provides for a method of developing antibody inhibitors of virus infection by immunization of animals with 10, 15, 20, or more consecutive amino acids of the polypeptides of SEQUENCE:10 or SEQUENCE:11 or SEQUENCE:20. Inventive methods further include the development of antibody fragments or humanized antibodies.

The invention still further provides for identifying target regions of LDLR polynucleotides. The invention also provides labeled probes for identifying LDLR polynucleotides by in situ hybridization.

The invention provides for the use of an LDLR inhibitor according to the invention to prepare a medicament for preventing or inhibiting HCV infection. The invention further provides for the use of an LDLR inhibitor according to the invention to prepare a medicament for preventing or inhibiting viral infection.

The invention further provides for directing an LDLR inhibitor to specific regions of the LDLR protein or at specific functions of the protein; in a preferred embodiment, the inhibitor will be directed to the region of the protein defined by the polypeptide of SEQUENCE:10.

The invention also provides a pharmaceutical composition for inhibiting expression of LDLR, comprising an antisense oligonucleotide according to the invention in a mixture with a physiologically acceptable carrier or diluent.

The invention further provides a ribozyme capable of specifically cleaving LDLR RNA, and a pharmaceutical composition comprising the ribozyme.

The invention also provides small molecule inhibitors of LDLR wherein the inhibitors are capable of reducing the activity of LDLR or of reducing or preventing the expression of LDLR mRNA.

The invention further provides for inhibitors of LDLR that modify specific functions of the protein other than acting as a receptor for low density lipoprotein, such functions including interaction with other proteins such as hepatitis C viral proteins including the HCV E2 protein.

The invention further provides for compounds that modulate LDLR trafficking to the endosome or lysosome and thereby reduce the effective concentration of LDLR at the plasma membrane.

The invention further provides for compounds that alter post-translational modifications of LDLR including but not limited to glycosylation, meristoylation, and phosphorylation.

The invention further provides a human genetic screening method for identifying a low density lipoprotein receptor gene mutation comprising: (a) treating, under amplification conditions, a sample of genomic DNA from a human with a polymerase chain reaction (PCR) primer pair for amplifying a region of human genomic DNA containing nucleotide position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of low density lipoprotein receptor gene, said treatment producing an amplification product containing said region; and (b) detecting in the amplification product of step (a) the presence of a nucleotide mutation as described by any one of SEQUENCE:5-9, SEQUENCE:14-19, or SEQUENCE:45-61, thereby identifying said mutation.

In certain embodiments of this method, the region comprises a nucleotide sequence represented by a sequence selected from the group consisting of: SEQUENCE:5-9, SEQUENCE:14-19, or SEQUENCE:45-61. Also provided is a method of detecting, wherein the detecting comprises treating, under hybridization conditions, the amplification product of step (a) above with an oligonucleotide probe specific for the point mutation, and detecting the formation of a hybridization product. In certain embodiments of the method, the oligonucleotide probe comprises a nucleotide sequence from the group consisting of SEQUENCE:5-9, SEQUENCE:14-19, or SEQUENCE:45-61 or some derivative thereof. Also provided is an isolated LDLR inhibitor selected from the group consisting of an antisense oligonucleotide, a ribozyme, a small inhibitory RNA (RNAi), a protein, a polypeptide, an antibody or antibody fragment, and a small molecule. The isolated inhibitor may be an antisense molecule or the complement thereof comprising at least 15 consecutive nucleic acids of the sequence of SEQUENCE:1, SEQUENCE:2, SEQUENCE:3, or SEQUENCE:4. In other embodiments, the isolated LDLR inhibitor (antisense molecule or the complement thereof) hybridizes under high stringency conditions to the sequence of SEQUENCE:1, SEQUENCE:2, SEQUENCE:3, or SEQUENCE:4.

The isolated LDLR inhibitor may be selected from the group consisting of an antibody and an antibody fragment. Also provided is a composition comprising a therapeutically effective amount of at least one LDLR inhibitor in a pharmaceutically acceptable carrier.

The invention also relates to a method of inhibiting the expression of LDLR in a mammalian cell, comprising administering to the cell an LDLR inhibitor selected from the group consisting of an antisense oligonucleotide, a ribozyme, a protein, an RNAi, a polypeptide, an antibody, and a small molecule.

The invention further relates to a method of inhibiting the expression of LDLR gene expression in a subject, comprising administering to the subject, in a pharmaceutically effective vehicle, an amount of an antisense oligonucleotide which is effective to specifically hybridize to all or part of a selected target nucleic acid sequence derived from the LDLR gene.

The invention still further relates to a method of preventing infection by a flavivirus, or other virus, in a human subject susceptible to the infection, comprising administering to the human subject an LDLR inhibitor selected from a group consisting of an antisense oligonucleotide, a ribozyme, an RNAi, a protein, a polypeptide, an antibody, and a small molecule, wherein said LDLR inhibitor prevents infection by said flavivirus.

The invention still further relates to a method of preventing or curing infection by a flavivirus or other virus in a human subject susceptible to the infection, comprising administering to the human subject an LDLR inhibitor selected from the group consisting of an antisense oligonucleotide, a ribozyme, an RNAi, a protein, a polypeptide, an antibody, and a small molecule, wherein the LDLR inhibitor prevents infection by the flavivirus or other virus and wherein the LDLR inhibitor is directed at one or more specific forms of the protein defined by a mutation at position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of NT_{—}011295.10.

The invention still further relates to a method of preventing or curing infection by a flavivirus or any other virus in a human subject susceptible to the infection by administering one of the polypeptides of the sequence: SEQUENCE:10, SEQUENCE:11, SEQUENCE:12, SEQUENCE:13, or SEQUENCE:20.

The invention still further relates to a method of preventing or curing infection by a flavivirus or any other virus in a human subject susceptible to the infection by administering a polypeptide composed of 5 or more consecutive amino acids of the sequence: SEQUENCE:10, SEQUENCE:11, SEQUENCE:12, SEQUENCE:13 or SEQUENCE:20.

The invention further relates to a method of identifying antiviral compounds by measuring the ability of the compound to bind to a polypeptide composed of 5, 10, 15, 20 or more consecutive amino acids of the sequence: SEQUENCE:10, SEQUENCE:11, or SEQUENCE:20.

The invention further relates to a method of identifying antiviral compounds by (a) measuring the ability of the compound to bind to a polypeptide composed of 5, 10, 15, 20 or more consecutive amino acids of the sequence: SEQUENCE:10, SEQUENCE:11 or SEQUENCE:20, and (b) subsequently testing the compound for its ability to inhibit virus infection, preferably RNA virus infection, preferably positive strand RNA virus infection, preferably flavivirus infection, most preferably hepatitis C virus infection. Preferred embodiments include but are not limited to the use of high-throughput screening methods or compounds from small molecule libraries, antibodies, antibody fragments, hybridoma libraries, or polypeptides composed of 5, 10, 15, 20 or more consecutive amino acids of the sequence: SEQUENCE:12 or SEQUENCE:13. Preferred embodiments further include but are not limited to the use of cytopathic and noncytopathic viruses, virus replicons, hybrid viruses, cytotoxicity assays, cell viability assays, reverse transcriptase polymerase chain reaction, TaqMan, and western blotting of viral proteins to assess the inhibition of virus infection, replication or pathogenicity.

The invention further relates to a method of identifying antiviral compounds by (a) measuring the ability of the compound to bind to a polypeptide composed of 5, 10, 15, 20 or more consecutive amino acids of the sequence SEQUENCE:10, SEQUENCE:11 or SEQUENCE:20 while (b) further measuring the ability of the compound to inhibit the binding of LDL or VLDL to the LDLR protein. Preferred embodiments include methods that permit the identification of antiviral compounds that bind the polypeptides of SEQUENCE:10, SEQUENCE:11, or SEQUENCE:20 while preserving the ability of the LDLR protein to bind and internalize LDL and VLDL. Preferred embodiments further involve the use of high-throughput screening methods and libraries of small molecule compounds, antibodies, antibody fragments, hybridoma libraries, or polypeptides composed of 5, 10, 15, 20 or more consecutive amino acids of the sequence: SEQUENCE:12 or SEQUENCE:13. Further preferred embodiments involve the use of cells expressing the polypeptide of SEQUENCE:20 on their cell surface whereby inhibition of LDL or VLDL binding is measured through the use of radiolabeled or fluorescently labeled LDL or VLDL. In still further embodiments, LDLR protein expression can be the result of endogenous or transgenic expression of the nucleic acid sequence of SEQUENCE:1, SEQUENCE:2, SEQUENCE:3, or SEQUENCE:4 or a component thereof. In a still further embodiment, cells in culture that normally express the LDLR protein on their surface, including but not limited to hepatocellular carcinoma derived cell lines or primary hepatocytes, may be used for measuring inhibition of VLDL or LDL binding to the LDLR protein. In a still further preferred embodiment, LDL or VLDL binding to the LDLR protein can be measured along with receptor mediated endocytosis, ligand release, and receptor recycling to the plasma membrane. In a still further embodiment, endocytosis, ligand release, and receptor recycling can be measured using radiolabeled, fluorescently labeled, or colloidal gold labeled LDL or VLDL and analytical methods such as scintillation counting, fluorescent microscopy, cell sorting, or electron microscopy.

Also provided is a method for inhibiting expression of a LDLR target gene in a cell in vitro comprising introduction of a ribonucleic acid (RNA) into the cell in an amount sufficient to inhibit expression of the LDLR target gene, wherein the RNA is a double-stranded molecule with a first strand consisting essentially of a ribonucleotide sequence which corresponds to a nucleotide sequence of the LDLR target gene and a second strand consisting essentially of a ribonucleotide sequence which is complementary to the nucleotide sequence of the LDLR target gene, wherein the first and the second ribonucleotide strands are separate complementary strands that hybridize to each other to form the double-stranded molecule, and the double-stranded molecule inhibits expression of the target gene.

In certain embodiments of the method, the first ribonucleotide sequence comprises at least 20 bases which correspond to the LDLR target gene and the second ribonucleotide sequence comprises at least 20 bases which are complementary to the nucleotide sequence of the LDLR target gene. In still further embodiments, the target gene expression is inhibited by at least 10%.

In still further embodiments of the method, the double-stranded ribonucleic acid structure is at least 20 bases in length and each of the ribonucleic acid strands is able to specifically hybridize to a deoxyribonucleic acid strand of the LDLR target gene over the at least 20 bases.

Also provided is the use of any of the proteins consisting of SEQUENCE:10 or SEQUENCE:11 as a component of a therapeutic composition.

In a further embodiment, a nucleic acid encoding the LDLR protein, LDLR mutant protein, or LDLR polypeptide can be administered in the form of gene therapy.

### BRIEF DESCRIPTION OF THE FIGURES

FIG. 1 (SEQUENCE:1) is a polynucleotide sequence consisting of the consecutive nucleotide bases at positions 2,460,001-2,509,020 of NCBI Accession No. NT_{—}011295.10, LDLR.

FIG. 2 is a Table showing the locations of 28 mutations in the LDLR gene (identified as SEQUENCE:5-9, SEQUENCE:14-19, and SEQUENCE:45-61), the allelic variants (base substitutions), genomic surrounding sequence, coordinates of the mutation on the genomic sequence, and NCBI dbSNP ID if any.

FIG. 3 shows polynucleotide SEQUENCE:2 consisting of an RNA fragment of LDLR, SEQUENCE:4 consisting of the entire mutant mRNA sequence of LDLR, and polypeptides SEQUENCE:10, SEQUENCE:11, and SEQUENCE:20 consisting of polypeptide fragments of and the full length LDLR protein. FIG. 3 also shows SEQUENCE:12 consisting of the APOB polypeptide and SEQUENCE:13 consisting of the APOE polypeptide. For nucleotide sequences SEQUENCE:2 and SEQUENCE:4, bold, singly underlined degenerate nucleotides are as denoted as follows: Y denotes C or U; R denotes A or G; K denotes G or U; M denotes A or C; S denotes G or C; W denotes A or T. For amino acid sequences SEQUENCE:10-13 and SEQUENCE:20, X denotes the amino acid variants as follows: SEQUENCE 10: amino acid position 391 is amino acid A or T; SEQUENCE 11: amino acid position 391 is amino acid A or T; SEQUENCE 20: amino acid position 391 is amino acid A or T.

FIG. 4 (SEQUENCE:3) is a polynucleotide sequence consisting of the consecutive nucleotide bases at positions 2,460,001-2,509,020 of NCBI Accession No. NT_{—}011295.10, LDLR with mutations of the present invention. For the nucleotide SEQUENCE:3, bold, singly underlined degenerate nucleotides are represented as follows: R denotes A or G; Y denotes C or T; K denotes G or T; M denotes A or C; S denotes G or C; and W denotes A or T. Bold, doubly underlined nucleotides represent the following mutations: SEQUENCE:3 position 2506013, nucleotide(s) is “-” (deleted nucleotide), and an alternate mutation nucleotides is G (inserted nucleotide). SEQUENCE:3 position 2506029-2506031 is CTA, and alternate mutation nucleotides is “---” (three nucleotide deletion).

### DETAILED DESCRIPTION OF THE INVENTION

### Introduction and Definitions

This invention relates to novel mutations in the low density lipoprotein receptor gene, use of these mutations for diagnosis of susceptibility or resistance to viral infection, to proteins encoded by a gene having a mutation according to the invention, and to prevention or inhibition of viral infection using the proteins, antibodies, and related nucleic acids. These mutations correlate with resistance of the carrier to infection with viruses, particularly RNA viruses, particularly positive strand RNA viruses, particularly flavivirus, most particularly hepatitis C virus.

Much of current medical research is focused on identifying mutations and defects that cause or contribute to disease. Such research is designed to lead to compounds and methods of treatment aimed at the disease state. Less attention has been paid to studying the genetic influences that allow people to remain healthy despite exposure to infectious agents and other risk factors. The present invention represents a successful application of a process developed by the inventors by which specific populations of human subjects are ascertained and analyzed in order to discover genetic variations or mutations that confer resistance to disease. The identification of a sub-population segment that has a natural resistance to a particular disease or biological condition further enables the identification of genes and proteins that are suitable targets for pharmaceutical intervention, diagnostic evaluation, or prevention, such as prophylactic vaccination.

We have previously described a method of identifying novel drug targets and developing pharmaceutical products through the identification of beneficial mutations that occur naturally in the human population (U.S. patent application Ser. No. 09/707,576). We describe here another target identified from studying hepatitis C infection.

As one skilled in the art will appreciate, many populations have evolved genetic mutations that confer resistance to infectious disease. Pathogens that cause significant morbidity and mortality in the target population negatively impact the reproductive success of susceptible individuals. Individuals who carry naturally occurring gene mutations that confer protection from infection escape negative selective pressures, and over time, their beneficial alleles are enriched in the overall population.

Using this principal as our starting point, we investigated the possibility that human populations carry gene mutations that confer resistance to the hepatitis C virus. The purpose of this investigation was to identify resistance-conferring mutations and develop drugs that mimic their antiviral effects in susceptible, virus-infected populations.

The sub-population segment identified herein is comprised of individuals who, despite repeated exposure to hepatitis C virus (HCV) have nonetheless remained sero-negative, while other cohorts have become infected (sero-positive). The populations studied included hemophiliac patients subjected to repeated blood transfusions, and intravenous drug users who become exposed through shared needles and other risk factors. By comparing the genetic make-up of serially exposed seronegative subjects to HCV seropositive control subjects, we have identified several mutations in the LDL receptor that confer resistance to HCV infection. These mutations inhibit virus infection in our seronegative cohort by preventing the virus from attaching to LDLR and invading the host cell.

The low density lipoprotein receptor (LDLR) is a widely expressed mammalian receptor that is a critical regulator of cholesterol metabolism. LDLR functions to remove cholesterol carrying liposomes from the circulation by receptor mediated endocytosis (Hussain, M M. Front Biosci. 6:D417-28, 2001; Willnow, T E, J Mol Med. 77(3):306-15, 1999). LDLR specifically binds to ligands containing apolipoprotein B (APOB) and apolipoprotein E (apoE), both of which serve as carries of serum cholesterol. Mutations in the LDLR are responsible for numerous forms of familial hypercholesterolemia (FH) (Hobbs, H H, et al. Hum Mutat. 1(6):445-66, 1992). FH is a dominant genetic disorder characterized by severe elevations in total serum cholesterol and LDL cholesterol resulting in premature atherosclerosis, early onset coronary artery disease, and death. To date more than 700 mutations have been identified in the LDL receptor that are responsible for inherited forms of hypercholesterolemia (http://www.ucl.ac.uk/fh/).

In view of this complex role of the LDLR gene, it is of significant interest that the present invention has identified a strong correlation between mutations in the LDLR gene, and resistance to HCV infection in carriers of these mutations. The present invention therefore will permit further elucidation of the role of LDLR in HCV viral entry, persistence, and resistance. The present invention further provides a method for treating HCV and related flaviviral infections by the development of therapeutic strategies designed to mimic the biochemical effects of LDLR resistance mutations. In reference to the detailed description and preferred embodiment, the following definitions are used:

A: adenine; C: cytosine; G: guanine; T: thymine (in DNA); and U: uracil (in RNA)

Allele: A variant of DNA sequence of a specific gene. In diploid cells a maximum of two alleles will be present, each in the same relative position or locus on homologous chromosomes of the chromosome set. When alleles at any one locus are identical the individual is said to be homozygous for that locus, and when they differ the individual is said to be heterozygous for that locus. Since different alleles of any one gene may vary by only a single base, the possible number of alleles for any one gene is very large. When alleles differ, one is often dominant to the other, which is said to be recessive. Dominance is a property of the phenotype and does not imply inactivation of the recessive allele by the dominant. In numerous examples the normally functioning (wild-type) allele is dominant to all mutant alleles of more or less defective function. In such cases the general explanation is that one functional allele out of two is sufficient to produce enough active gene product to support normal development of the organism (i.e., there is normally a two-fold safety margin in quantity of gene product).

Haplotype: One of many possible pluralities of Alleles, serially ordered by chromosomal localization and representing that set of Alleles carried by one particular homologous chromosome of the chromosome set.

Nucleotide: A monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose it is referred to as a nucleotide. A sequence of operatively linked nucleotides is typically referred to herein as a “base sequence” or “nucleotide sequence”, and their grammatical equivalents, and is represented herein by a formula whose left to right orientation is in the conventional direction of 5′-terminus to 3′-terminus.

Base Pair (bp): A partnership of adenine (A) with thymine (T), or of cytosine (C) with guanine (G) in a double stranded DNA molecule. In RNA, uracil (U) is substituted for thymine.

Nucleic Acid: A polymer of nucleotides, either single or double stranded.

Polynucleotide: A polymer of single or double stranded nucleotides. As used herein “polynucleotide” and its grammatical equivalents will include the full range of nucleic acids. A polynucleotide will typically refer to a nucleic acid molecule comprised of a linear strand of two or more deoxyribonucleotides and/or ribonucleotides. The exact size will depend on many factors, which in turn depends on the ultimate conditions of use, as is well known in the art. The polynucleotides of the present invention include primers, probes, RNA/DNA segments, oligonucleotides or “oligos” (relatively short polynucleotides), genes, vectors, plasmids, and the like.

Gene: A nucleic acid whose nucleotide sequence codes for an RNA or polypeptide. A gene can be either RNA or DNA.

Duplex DNA: A double-stranded nucleic acid molecule comprising two strands of substantially complementary polynucleotides held together by one or more hydrogen bonds between each of the complementary bases present in a base pair of the duplex. Because the nucleotides that form a base pair can be either a ribonucleotide base or a deoxyribonucleotide base, the phrase “duplex DNA” refers to either a DNA-DNA duplex comprising two DNA strands (ds DNA), or an RNA-DNA duplex comprising one DNA and one RNA strand.

Complementary Bases: Nucleotides that normally pair up when DNA or RNA adopts a double stranded configuration.

Complementary Nucleotide Sequence: A sequence of nucleotides in a single-stranded molecule of DNA or RNA that is sufficiently complementary to that on another single strand to specifically hybridize to it with consequent hydrogen bonding.

Conserved: A nucleotide sequence is conserved with respect to a preselected (reference) sequence if it non-randomly hybridizes to an exact complement of the preselected sequence.

Hybridization: The pairing of substantially complementary nucleotide sequences (strands of nucleic acid) to form a duplex or heteroduplex by the establishment of hydrogen bonds between complementary base pairs. It is a specific, i.e. non-random, interaction between two complementary polynucleotides that can be competitively inhibited.

Nucleotide Analog: A purine or pyrimidine nucleotide that differs structurally from A, T, G, C, or U, but is sufficiently similar to substitute for the normal nucleotide in a nucleic acid molecule.

DNA Homolog: A nucleic acid having a preselected conserved nucleotide sequence and a sequence coding for a receptor capable of binding a preselected ligand.

Upstream: In the direction opposite to the direction of DNA transcription, and therefore going from 5′ to 3′ on the non-coding strand, or 3′ to 5′ on the mRNA.

Downstream: Further along a DNA sequence in the direction of sequence transcription or read out, that is traveling in a 3′- to 5′-direction along the non-coding strand of the DNA or 5′- to 3′-direction along the RNA transcript.

Stop Codon: Any of three codons that do not code for an amino acid, but instead cause termination of protein synthesis. They are UAG, UAA and UGA and are also referred to as a nonsense or termination codon.

Reading Frame: Particular sequence of contiguous nucleotide triplets (codons) employed in translation. The reading frame depends on the location of the translation initiation codon.

Intron: Also referred to as an intervening sequence, a noncoding sequence of DNA that is initially copied into RNA but is cut out of the final RNA transcript.

Resistance: As used herein with regard to viral infection, resistance specifically includes all degrees of enhanced resistance or susceptibility to viral infection as observed in the comparison between two or more groups of individuals.

In one example of a human genetic screening method for identifying a low density lipoprotein receptor gene (LDLR) mutation comprising detecting in a nucleic acid sample the presence or absence of at least one LDLR mutation selected from the group consisting of: substitution of a non-reference nucleotide for a reference nucleotide at nucleotide position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506056, and 2506062; a base insertion at nucleotide position 2506013; or a three-base deletion at nucleotide position 2506029-2506031, the term “reference nucleotide” is understood to mean with reference to NT_{—}011295.1 or SEQUENCE:1. By “non-reference nucleotide” is understood to mean any nucleotide(s) other than the reference nucleotide at that position, including but not limited to mutations as described in SEQUENCES:5-9, 14-19, and 45-61.

Modes of Practicing the Invention

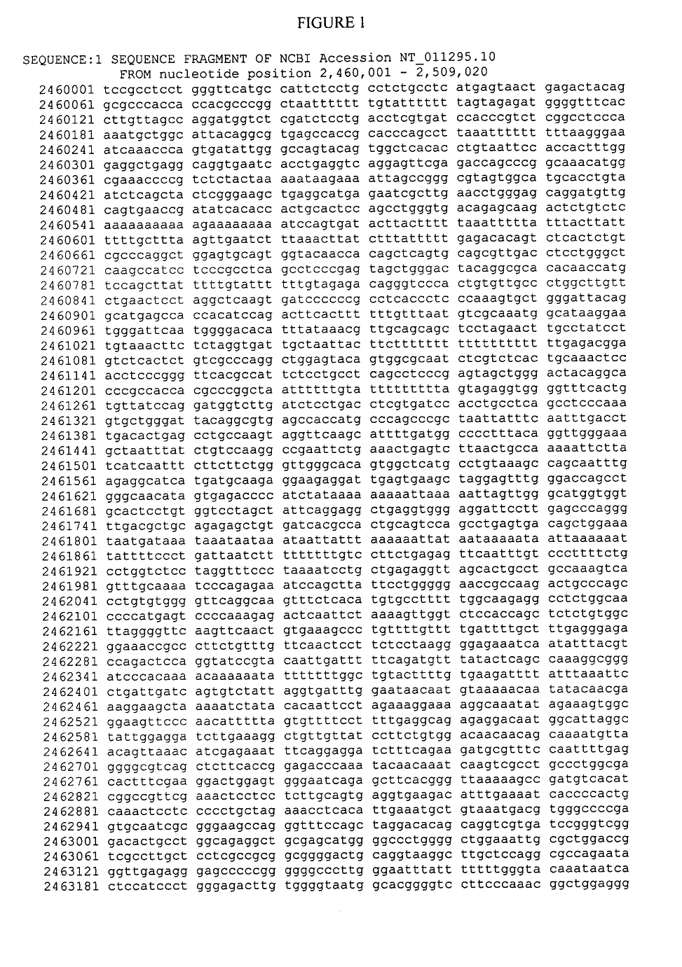

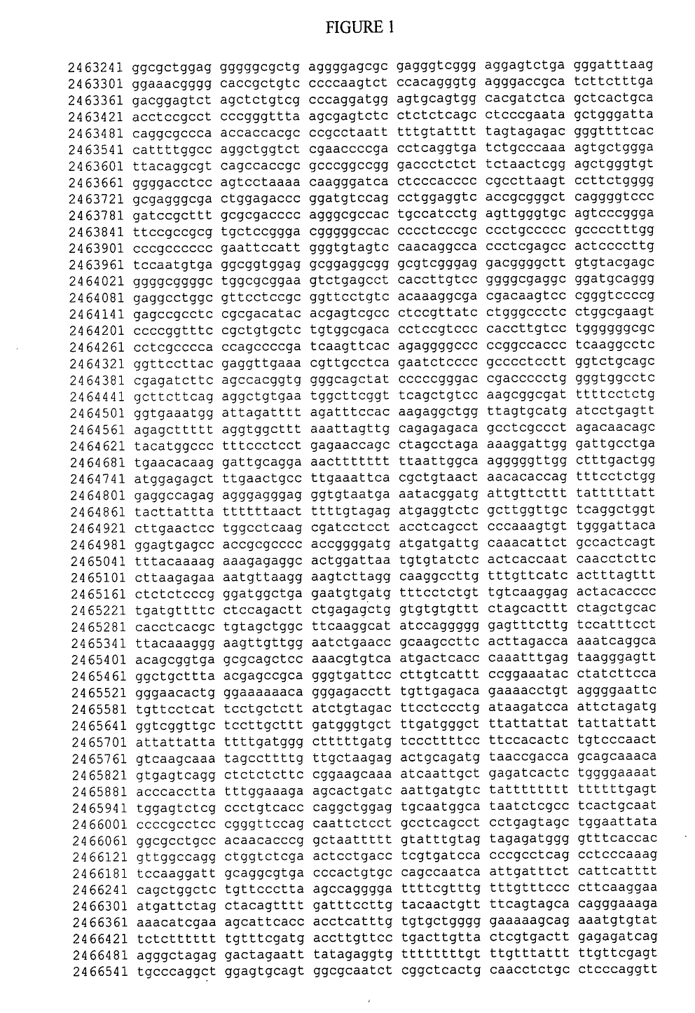

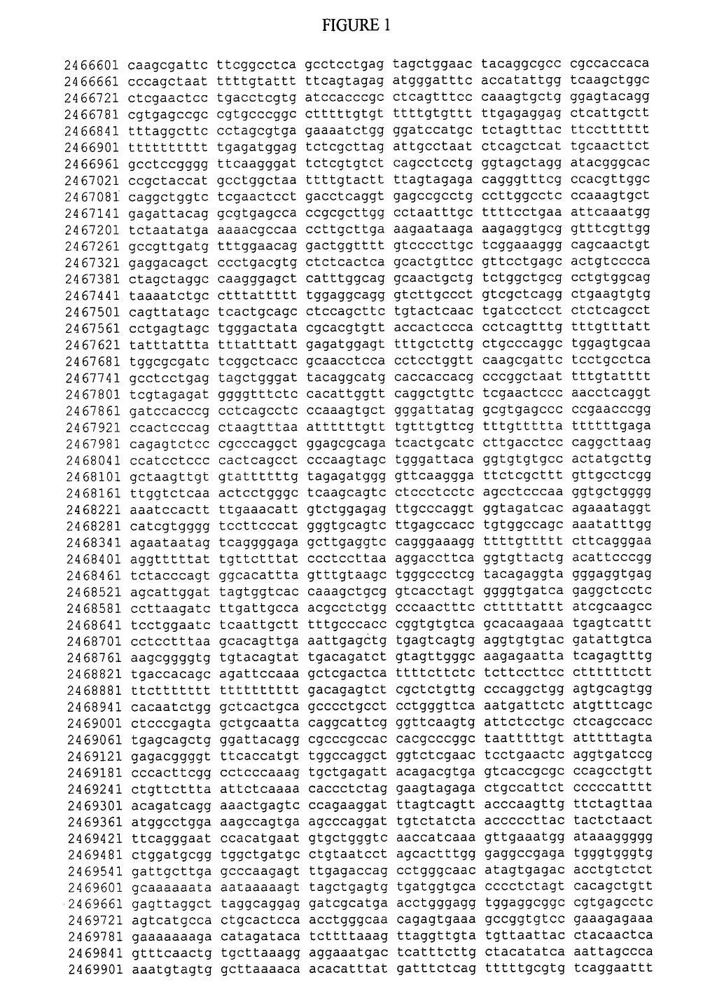

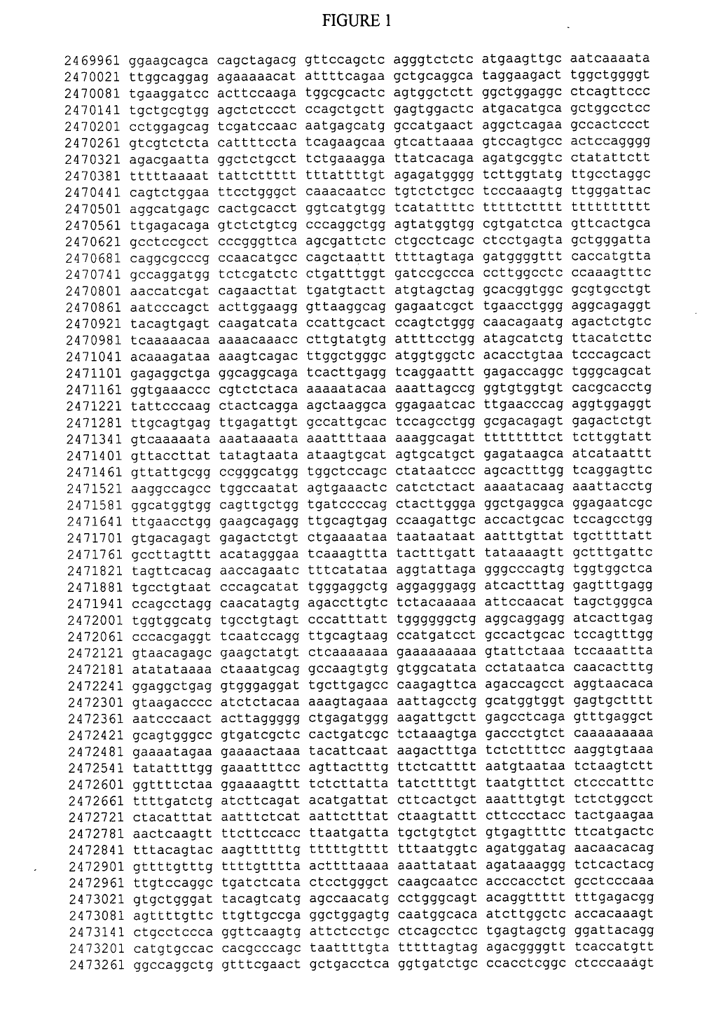

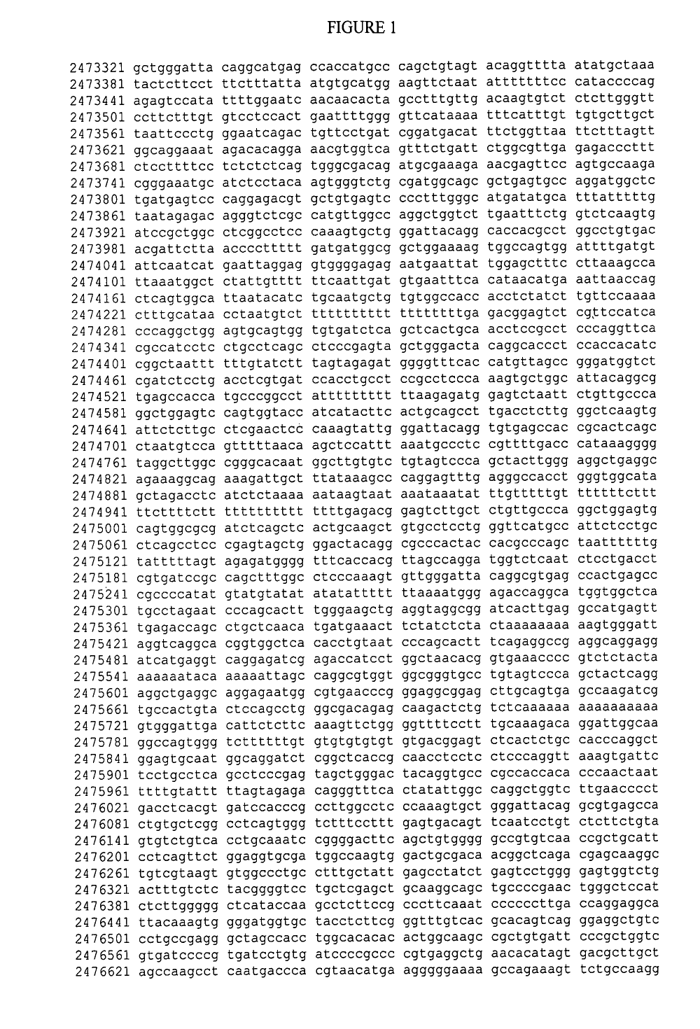

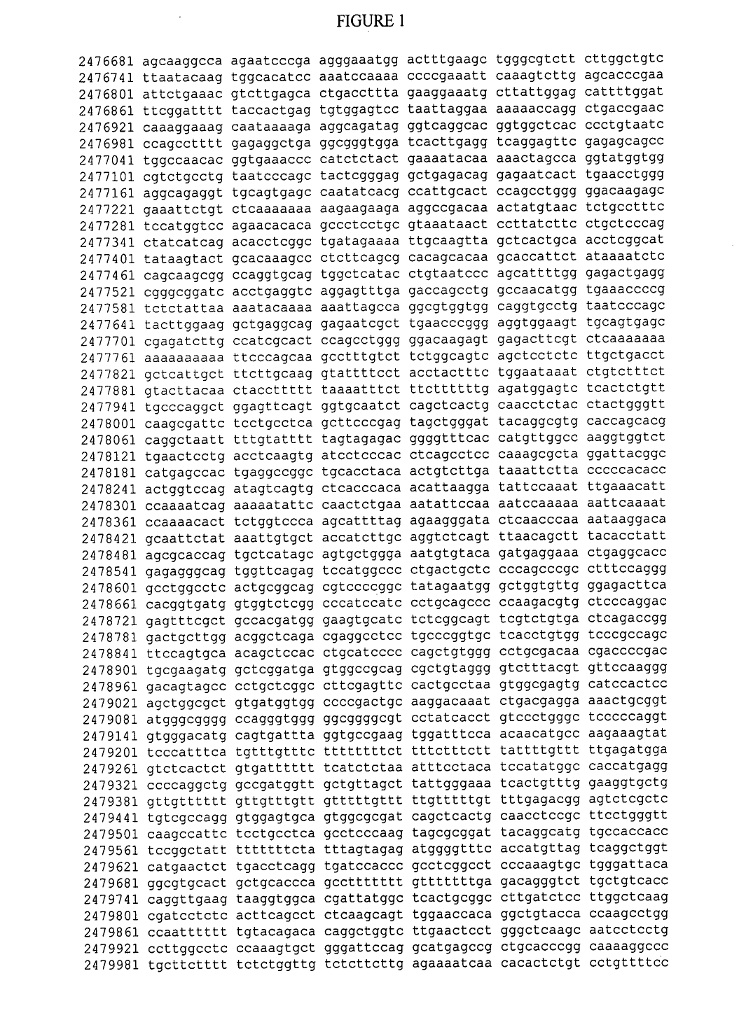

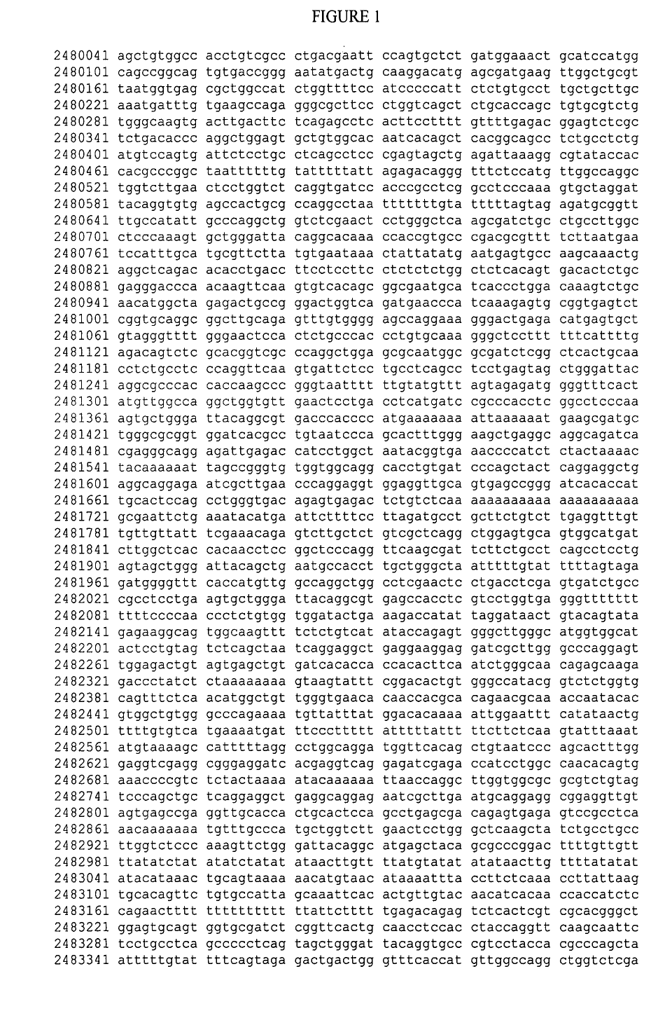

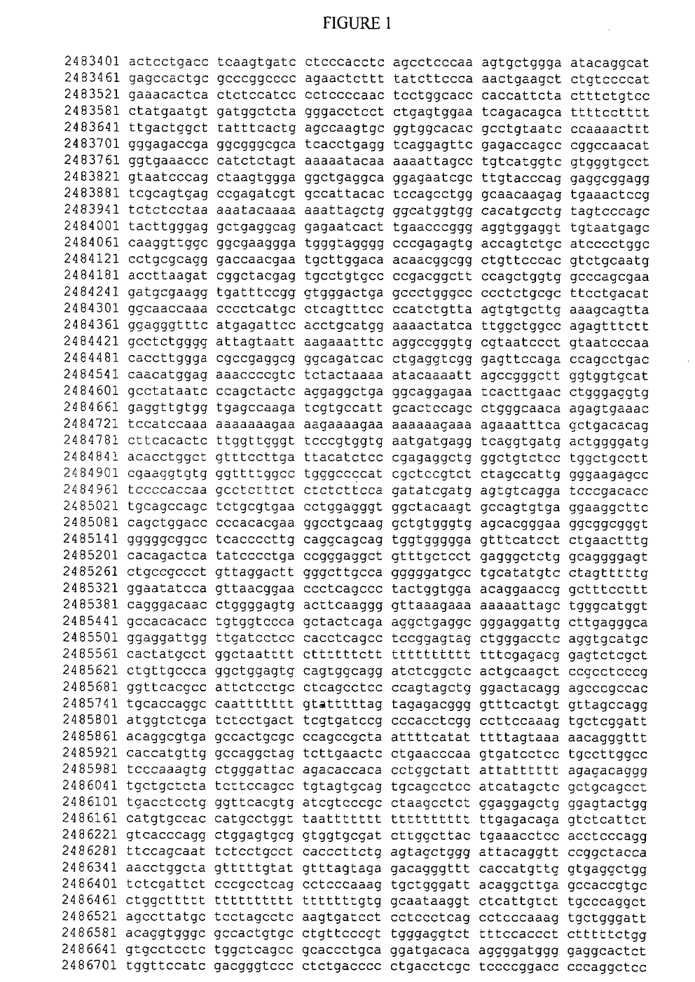

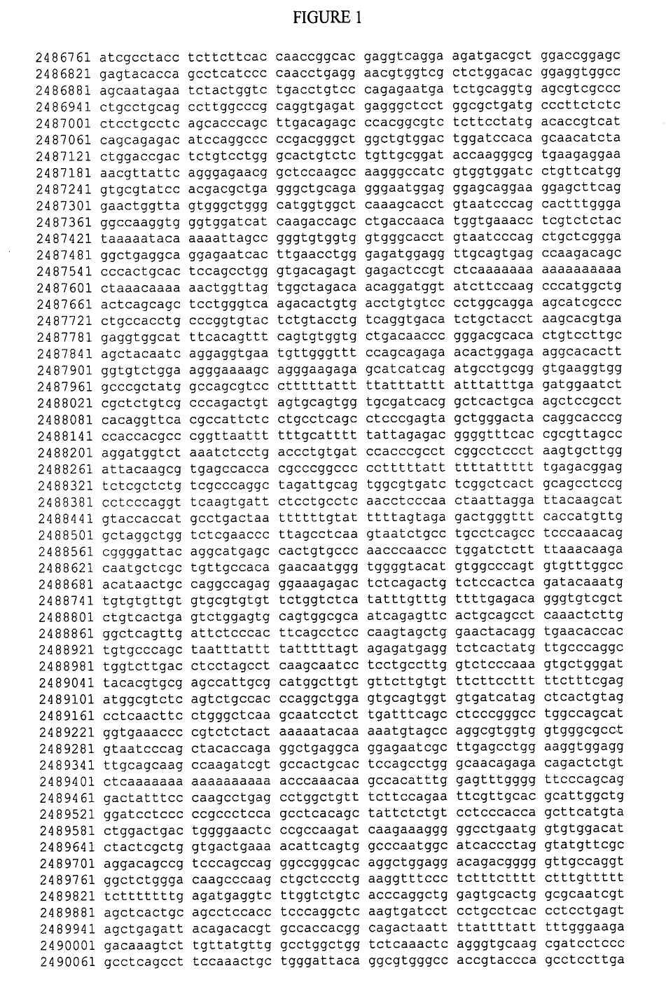

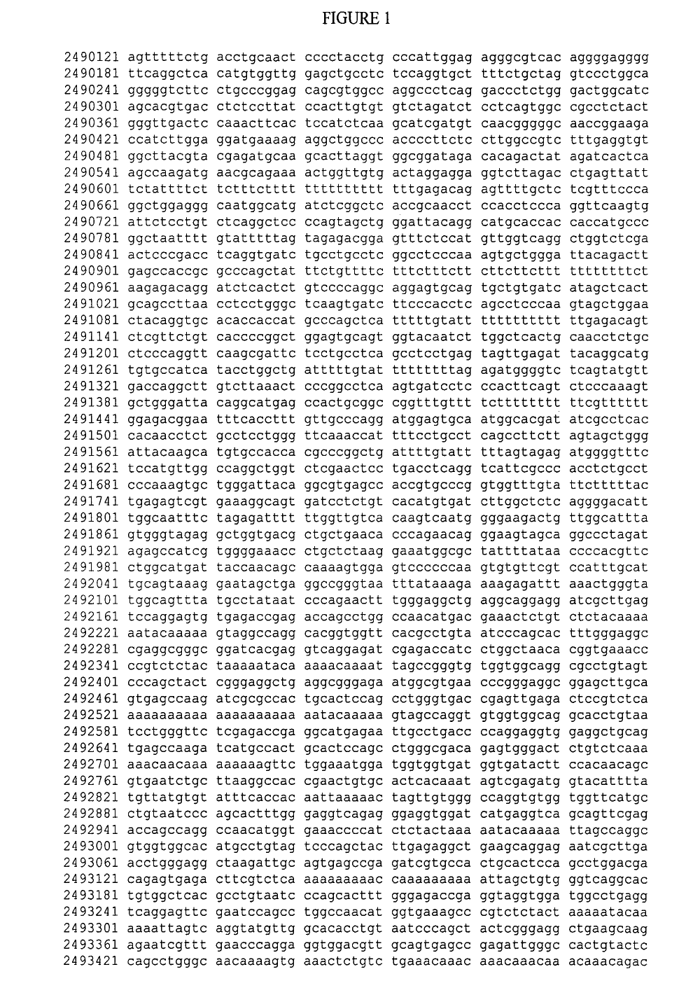

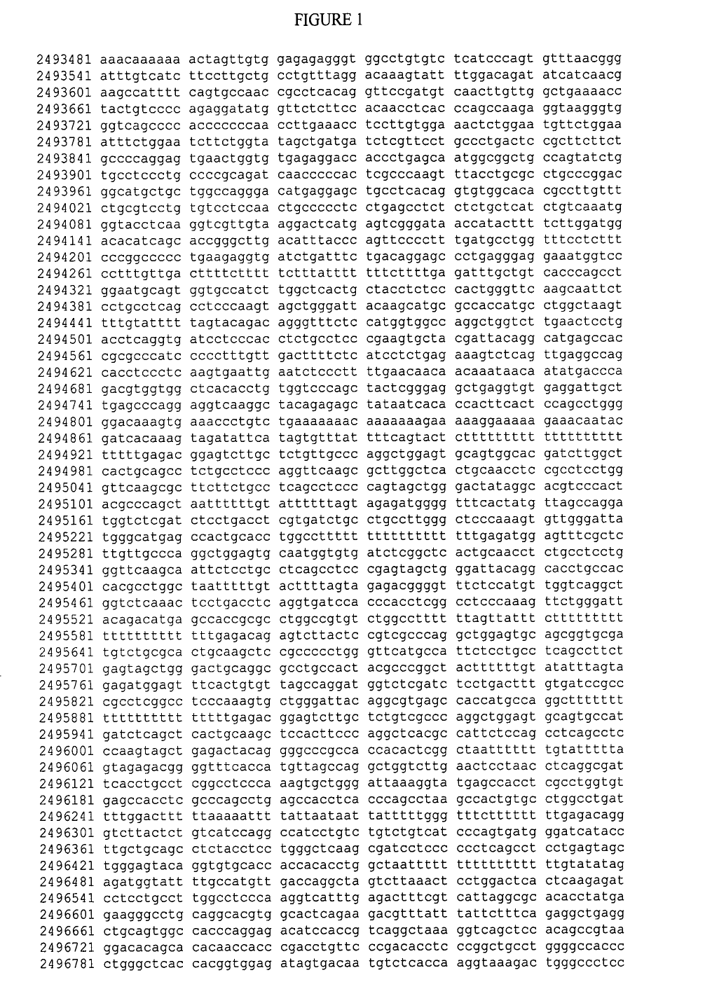

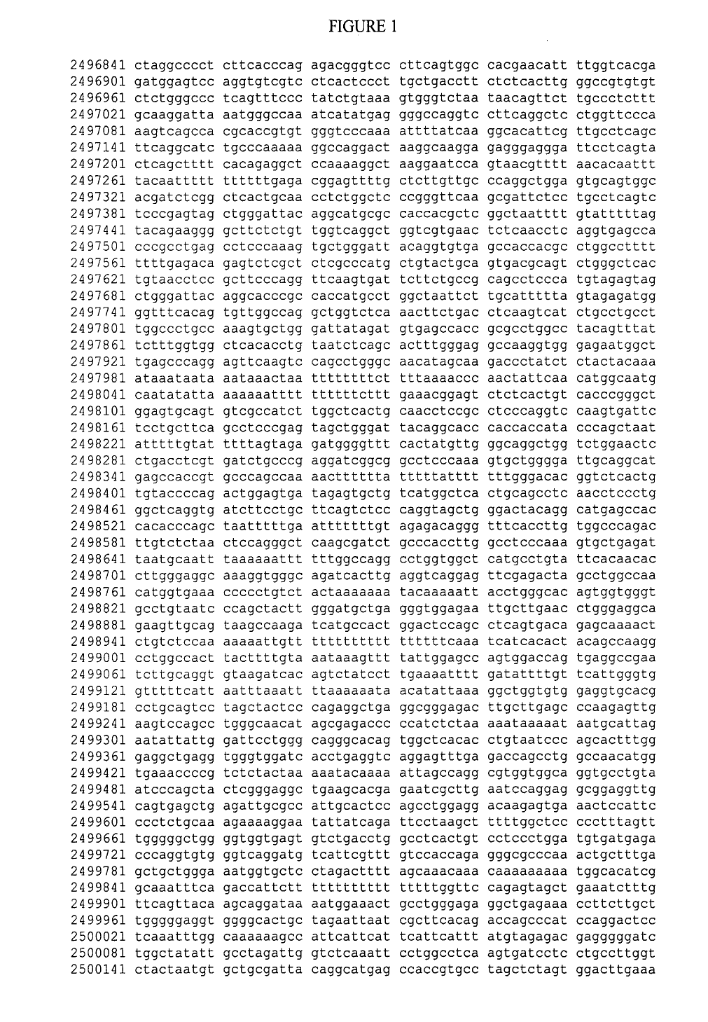

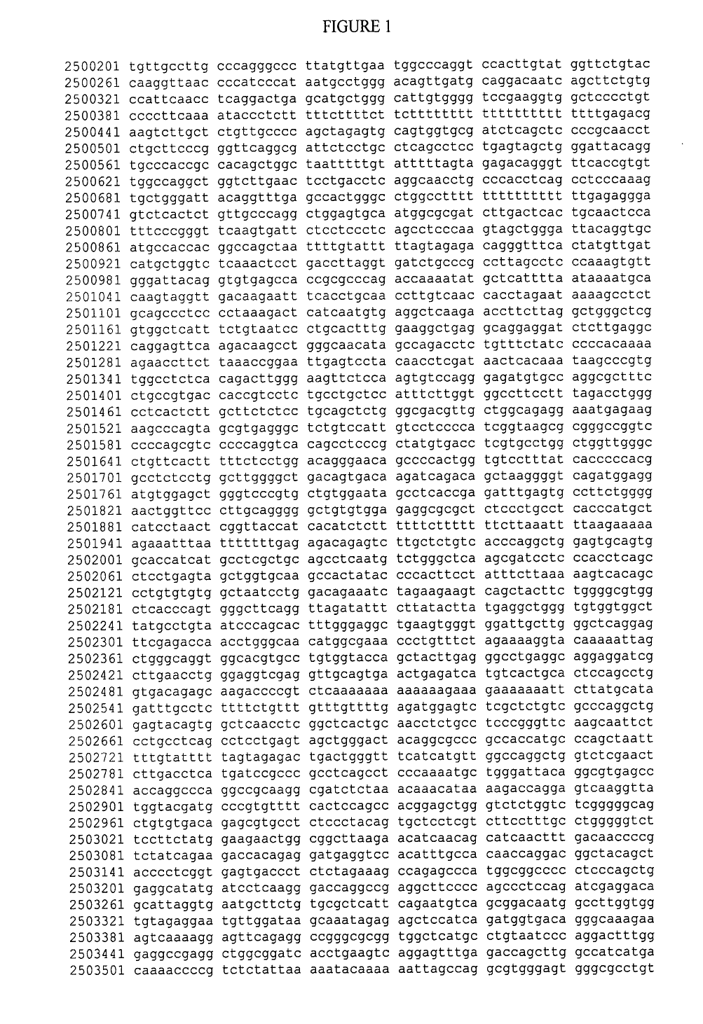

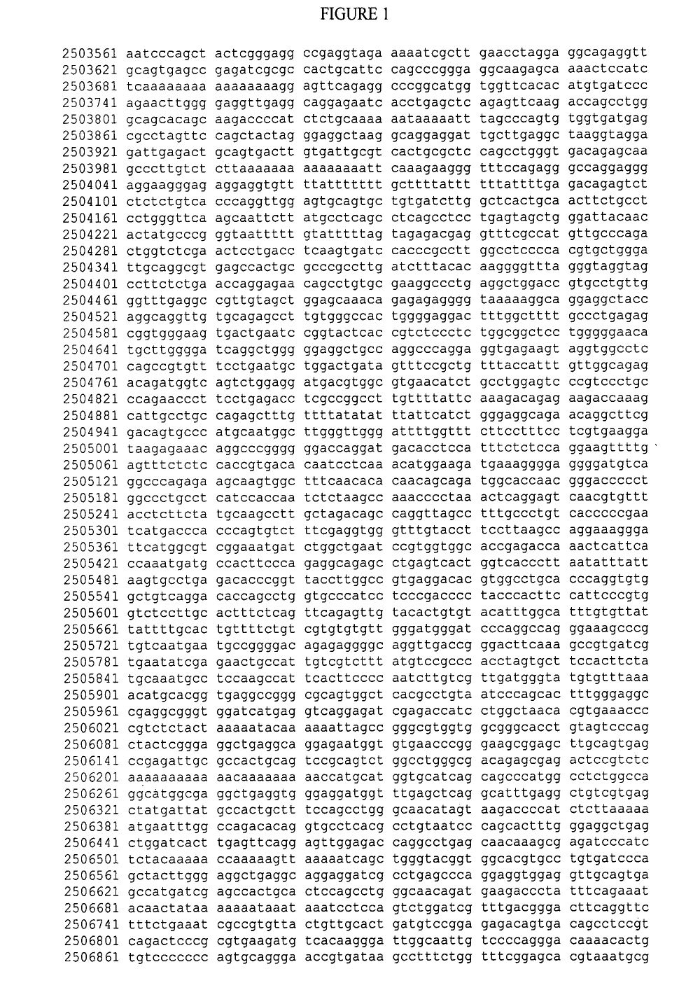

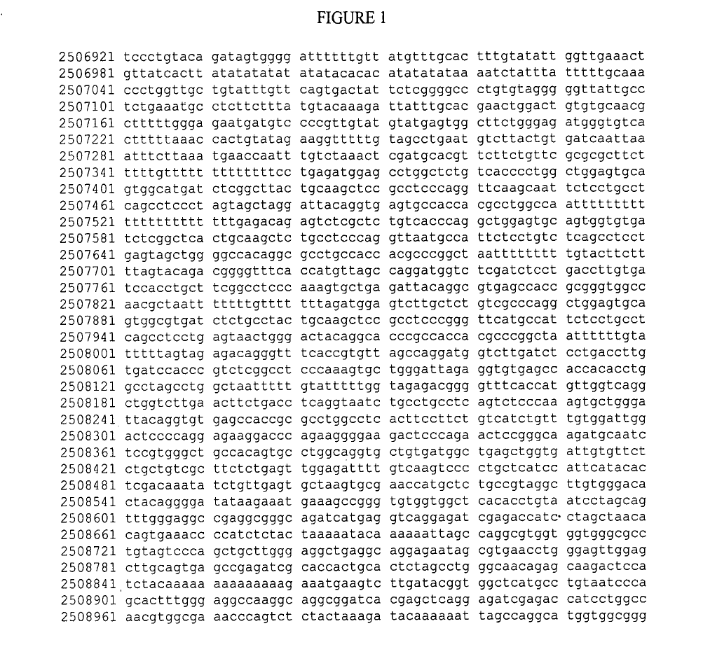

As known to those skilled in the art, multiple experimental and analytical approaches are applied to the study design of the present invention. Without limiting the scope of the present invention, several preferred modes are presented below and in the Examples. The present invention provides a novel method for screening humans for low density lipoprotein receptor alleles and haplotypes associated with resistance to infection by a virus, particularly a flavivirus, most particularly hepatitis C. The invention is based on the discovery that such resistance is associated with the particular base(s) encoded at a site of mutation (as further described herein) in the low density lipoprotein receptor gene DNA sequence at nucleotide position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of Genbank Accession No. NT_{—}011295.10 (consecutive bases 2,460,001-2,509,020 of which are provided as SEQUENCE:1 in FIG. 1), which encodes the human LDLR gene.

This invention discloses the results of a study that identified populations of subjects resistant or partially resistant to infection with the hepatitis C virus (HCV) and that further identified genetic mutations that confer this beneficial effect. Several genetic mutations in the low density lipoprotein receptor gene are identified, that are significantly associated with resistance to HCV infection. The study design used was a case-control, allele association analysis. Cases had serially documented or presumed exposure to HCV, but did not develop infection as documented by the development of antibodies to the virus (i.e. HCV seronegative). Control subjects were serially exposed subjects who did seroconvert to HCV positive. Case and control subjects were recruited from three populations, hemophilia patients from Vancouver, British Columbia, Canada; hemophilia patients from Northwestern France; and injecting drug users from the Seattle metropolitan region.

Case and control definitions differed between the hemophilia and IDU groups and were based upon epidemiological models of infection risk published in the literature and other models developed by the inventors, as described herein. For the hemophilia population, control subjects were documented to be seropositive for antibodies to HCV using commercial diagnostics laboratory testing. Case subjects were documented as being HCV seronegative, having less than 5% of normal clotting factor, and having received concentrated clotting factors before January 1987. Control injecting drug users were defined as documented HCV seropositive. Case injecting drug users were defined as documented HCV seronegative, having injected drugs for more than ten years, and having reported engaging in one or more additional risk behaviors. Additional risk behaviors include the sharing of syringes, cookers, or cottons with another IDU. 47 cases and 115 controls were included in this study population.

Selection of case and control subjects was performed essentially as described in U.S. patent application Ser. No. 09/707,576 using the population groups at-risk affected (“controls”) and at-risk unaffected (“cases”).

The present inventive approach to identifying gene mutations associated with resistance to HCV infection involved the selection of candidate genes. Approximately 50 candidate genes involved in viral binding to the cell surface, viral propagation within the cell, the interferon response, and aspects of the innate immune system and the antiviral response, were interrogated. Candidate genes were sequenced in cases and controls by using the polymerase chain reaction to amplify target sequences from the genomic DNA of each subject. PCR products from candidate genes were sequenced directly using automated, fluorescence-based DNA sequencing and an ABI3730 automated sequencer.

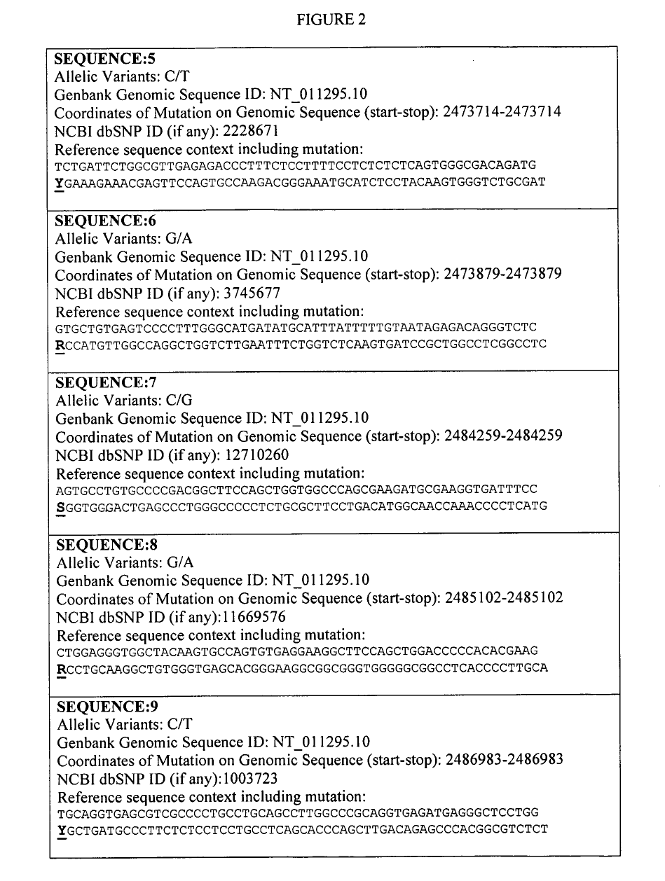

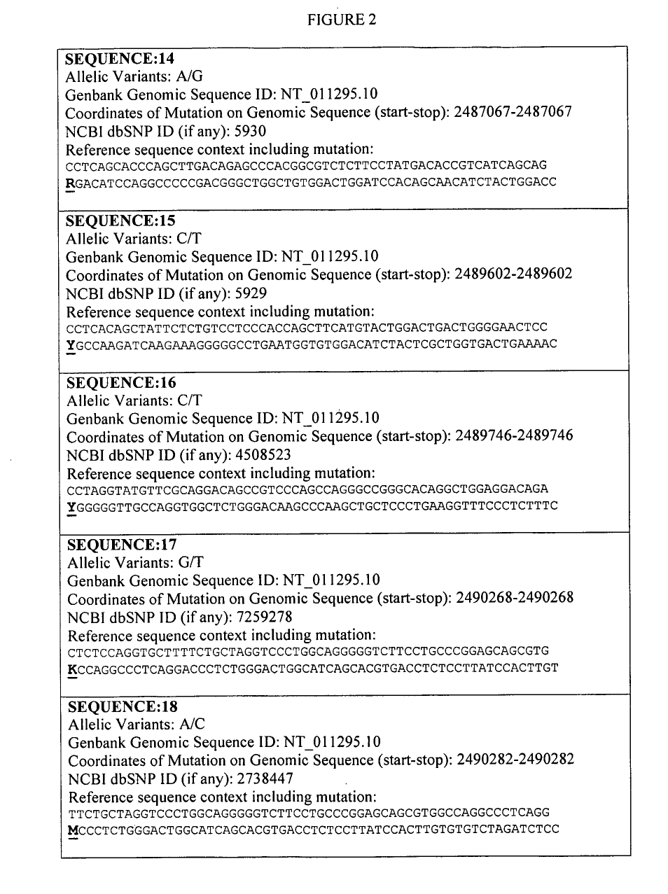

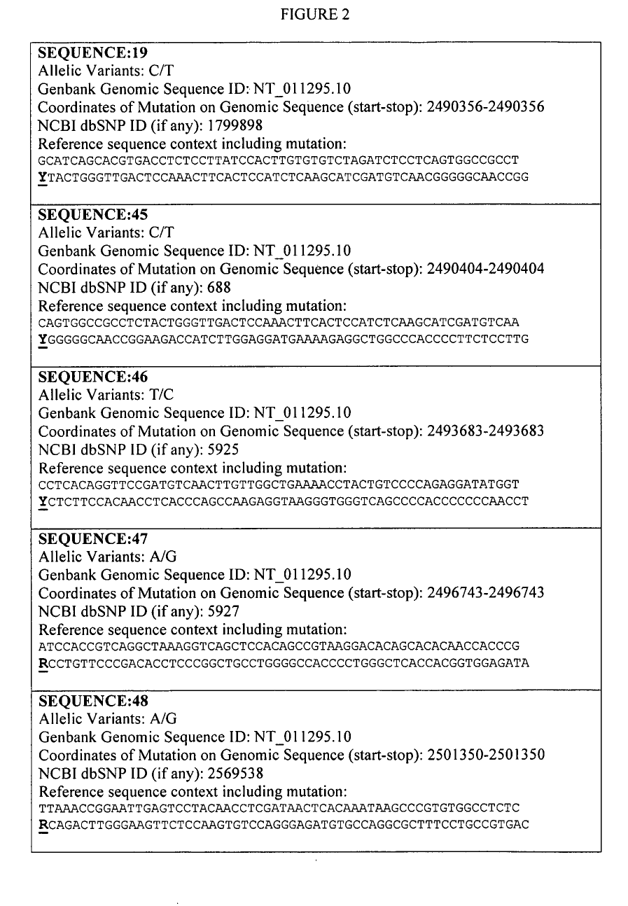

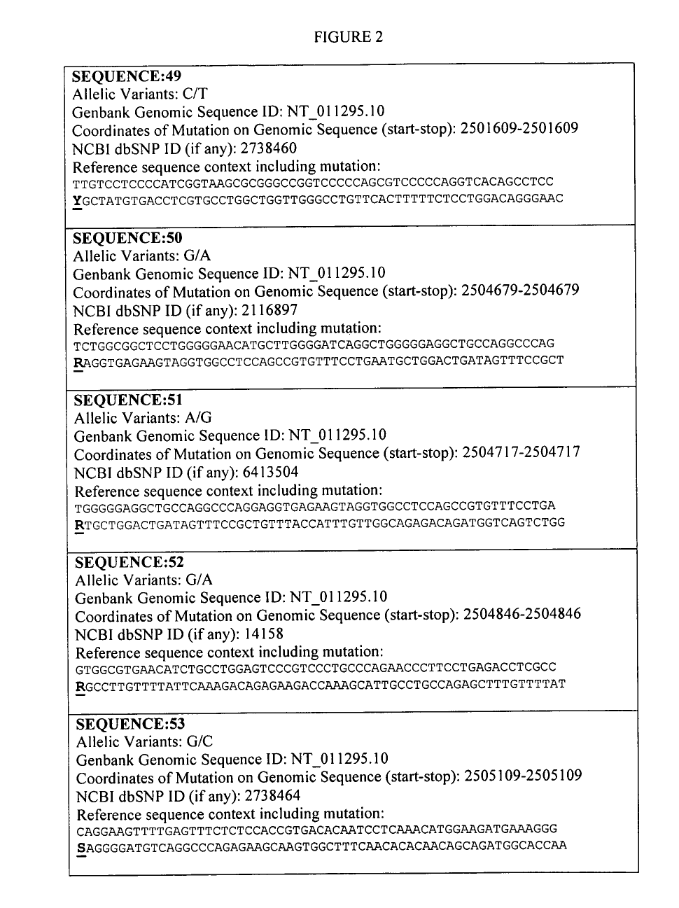

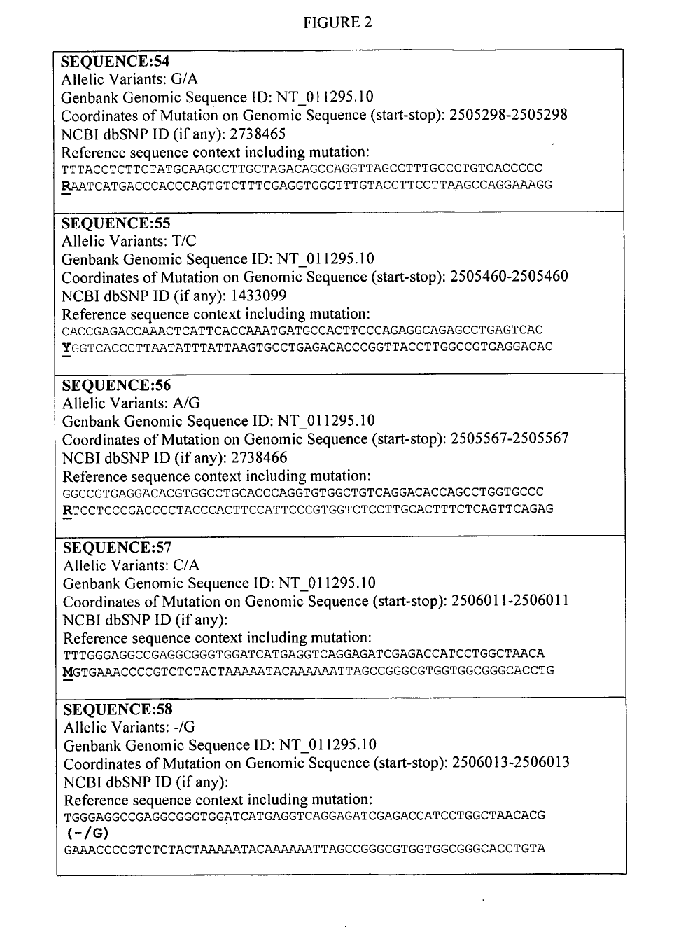

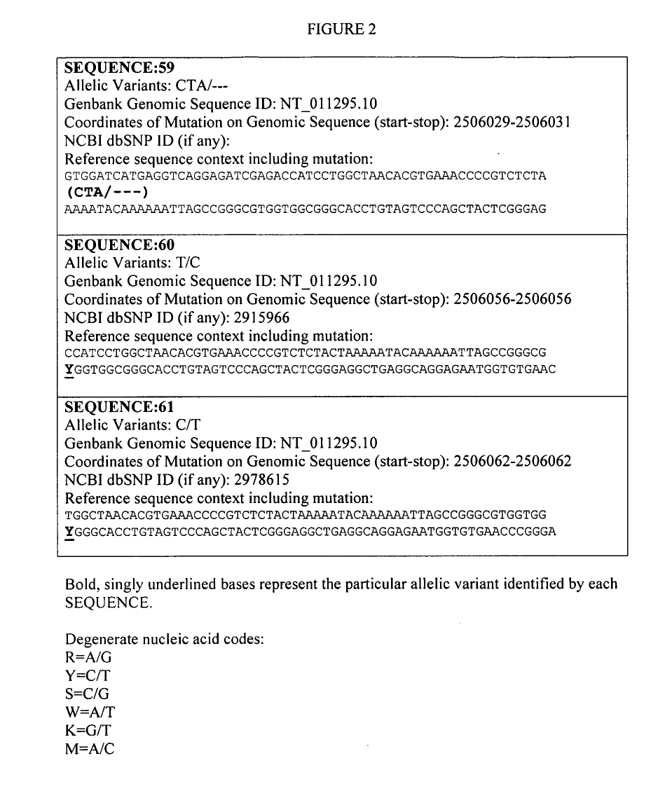

Exhaustive sequencing of the coding and regulatory regions of the low density lipoprotein receptor gene (LDLR) in the present population identified 28 polymorphic mutations occurring more than once. These mutations are characterized and identified in FIG. 2 as SEQUENCE:5-9, SEQUENCE:14-19, and SEQUENCE:45-61. Variant forms of the LDLR gene are produced by the presence of one or more of these 28 mutations. As further described below, resistance to HCV infection in the present population was found to be significantly associated (p<0.05) with distinct subsets of this group of mutations. Therefore, variant forms of the LDLR gene are believed to confer resistance to viral infection. In one preferred mode of numerical analysis, allele association analysis is performed to identify bias in the frequency of occurrence of a particular allele at one or more sites of mutation with respect to either the case or control group, thereby identifying one or mutations associated with resistance to HCV infection. This association is tested for statistical significance using any of a number of accepted statistical tests known to those skilled in the art, including chi-square analysis.

In another preferred mode of numerical analysis, linkage disequilibrium analysis as known to those skilled in the art is performed to identify predictive relationships between pluralities of mutations in the genotype data.

In another preferred mode of numerical analysis, haplotypes comprising combinatorial subsets of LDLR mutations are computationally inferred by Expectation Maximation (EM) methods as known to those skilled in the art (Excoffier, L et. al. Mol Biol Evol., 12(5):921-7, 1995). A number of haplotypes are identified in the case and control population by this analysis. Using this method, each subject in the population is assigned two parental haplotypes. Haplotype distributions among case and control subjects are analyzed by known statistical methods (including chi-square analysis) to identify bias toward either other group, thereby identifying particular haplotype that confer resistance to HCV infection.

In other preferred modes of analysis, specific genetic models of resistance to HCV infection are examined utilizing mutation allele data or inferred haplotype data (as described above). Exemplary genetic models include those that model resistance as dominant, additive, and recessive effects. Models are tested for their ability to significantly predict resistance to HCV infection by any one of a number of accepted statistical approaches, including without limitation, logistic regression.

Specific haplotypes or allelic states at one or more sites of mutation that are shown to be significantly associated with resistance to HCV infection by any of the above analytical approaches are further analyzed to identify biological effectors of the resistance. Such further analysis includes both computational and experimental modes of analysis. In one such further preferred embodiment, the haplotype identified as associated with resistance to HCV infection (a “resistant haplotype”) is compared with its nearest “neighbors” in terms of total mutational content. Such comparison identifies particular mutational states at specific sites within the gene that act to confer resistance. In another preferred embodiment, further population genotyping analysis is conducted in other portions of the LDLR gene and surrounding genomic region, including without limitation the introns, in order to identify additional mutations that are either independently associated with resistance to HCV infection or that contribute to more expansive haplotypes associated with resistance to HCV infection. In another preferred embodiment, a “resistant haplotype” is experimentally analyzed in comparison with related neighbors to identify biological differences that confer resistance. Such experimental analysis includes, without limitation, comparative analysis of expression levels, transcription of variant mRNAs, identification of exonic and intronic splice enhancers, and mRNA stability by methods as described elsewhere herein and as known to those skilled in the art. In one such embodiment, the comparative analyses are performed between samples derived from homozygous individuals carrying the resistant haplotype and one or more samples derived from individuals carrying other haplotypes for comparison.

As further described in Examples 6-8, particular haplotypes were determined to be significantly associated with resistance to HCV infection. Thus the invention provides genetic haplotypes that are resistant to HCV infection. As described further below, the mutations in these haplotypes are used to screen human subjects for resistance to viral infection, particularly flavivirus infection, most particularly hepatitis C infection. The invention further provides one or more specific regions of LDLR (as described below) that are targets for therapeutic intervention in viral infection, particularly flavivirus infection, most particularly HCV infection. Furthermore, the invention also provides novel forms of LDLR that are resistant to viral infection, particularly flavivirus infection, most particularly HCV infection.

We note that one of the mutations (as described by SEQUENCE:8) contained within our resistance haplotypes results in a non-conservative change in amino acid 391 of the native LDLR protein. This change substitutes a threonine for an alanine (A391T) in the sequence at this location. Alanine at position 391 is highly conserved in all mammalian species, which have either an alanine or the similarly aliphatic and non-polar valine at this position. The mutation identified in SEQUENCE:8 results in a non-conservative substitution of the chemically polar threonine at this location.

The mutation identified in SEQUENCE:8, like many of the mutations contributing to identified HCV resistance haplotypes, occurs within the epidermal growth factor (EGF) precursor homology domain of LDLR, a region responsible for receptor recycling and ligand release in the acidic endosomal compartment (Hussain, M M. Front Biosci. 6:D417-28, 2001; Rudenko, G, et al. Science. 298(5602):2353-8, 2002). While mutations in this region typically do not inhibit ligand binding to the receptor, receptor mediated endocytosis, ligand release, and receptor recycling are impaired. The concentration of resistance mutations in this specific and localized region of LDLR suggests that interactions between the virus and this domain are required for viral propagation in the host. The invention provides for therapeutic interventions that specifically target this region. The invention further provides for the use of this specific region as defined in SEQUENCE:10 as a therapeutic product.

Nearly every amino acid change in the LDLR has been demonstrated to result in some form of receptor dysfunction (Hobbs, H H, et al. Hum Mutat. 1(6):445-66, 1992). A391T, originally identified as an LDLR variant in an Afrikaner familial hypercholesterolemic population, is enriched in several FH populations throughout Europe (Kotze, M J, et al. S Afr Med J. 76(8):399-401, 1989; Kotze, M J, et al. S Afr Med J. 76(8):402-5, 1989; Kotze, M J, et al. J Med Genet. 26(4):255-9, 1989; Schuster, H, et al. Clin Genet. 38(6):401-9, 1990; Miserez, A R, et al. Am J Hum Genet. 52(4):808-26, 1993; Humphries, S, et al. J Med Genet. 30(4):273-9, 1993; Brink, P A, et al. Hum Genet. 77(1):32-5, 1987; Wang, J, et al. Hum Mutat. 18(4):359, 2001; Dedoussis, G V, et al. Hum Mutat. 23(3):285-6, 2004). Nevertheless, its effect on serum cholesterol is apparently mild, resulting in as little as a 10% increase in LDL-C and apolipoprotein B in the serum of heterozygous carriers (Gudnason, V, et al. Clin Genet. 47(2):68-74, 1995). This mutation has also been associated with an increase in ischemic stroke risk in one population (Frikke-Schmidt, R, et al. Eur Heart J. 25(11):943-951, 2004). The relatively mild nature of the resulting phenotype suggests that pharmaceutical products that faithfully replicate or mimic the biochemical effects of this mutation in vivo will have little or no hyperlipidemia-promoting character.

Knowledge of the fact that many naturally occurring HCV resistance-related mutations occur within the EGF precursor homology domain suggests that HCV and similar viruses require an interaction with this region in order to enter the cell and develop a stable infection. This knowledge also suggests that pharmaceutical products that directly target this region or the interaction of the virus with this region will successfully contribute to viral clearance. Finally, because mutation in this region has a very mild in vivo phenotype—carriers have no observable phenotype other than resistance to HCV infection—drugs that accurately mimic these mutational effects are expected to be free of significant negative side effect.

Other mutations observed to contribute to HCV infection-resistant haplotypes include mutations in the 3′-untranslated region (3′-UTR) of the LDLR gene and the portion of the LDLR gene encoding the ligand-binding domain R1. The concentration of mutations in these regions suggests additional mechanisms contribute to HCV resistance, including without limitation, mRNA stability, splicing control, and expression control. These regions therefore are targets for either genetic screening or therapeutic invention as described elsewhere.

The invention provides for genetic mutations of the human low density lipoprotein receptor gene, associated mRNA transcripts and proteins. The invention also discloses utility for the mutations, mRNA transcripts and proteins. These genetic mutations in LDLR confer on carriers a level of resistance to the hepatitis C virus and associated flaviviruses including but not limited to the West Nile virus, dengue viruses, yellow fever virus, tick-borne encephalitis virus, Japanese encephalitis virus, St. Louis encephalitis virus, Murray Valley virus, Powassan virus, Rocio virus, louping-ill virus, Banzi virus, Ilheus virus, Kokobera virus, Kunjin virus, Alfuy virus, bovine diarrhea virus, and the Kyasanur forest disease virus. Mutant LDLR cDNA is cloned from human subjects who are carriers of these mutations. Cloning is carried out by standard cDNA cloning methods that involve the isolation of RNA from cells or tissue, the conversion of RNA to cDNA, and the conversion of cDNA to double-stranded DNA suitable for cloning. As one skilled in the art will recognize, all of these steps are routine molecular biological analyses. Other methods include the use of reverse transcriptase PCR, 5′RACE (Rapid Amplification of cDNA Ends), or traditional cDNA library construction and screening by Southern hybridization. All mutant LDLR alleles described herein are recovered from patient carriers. Each newly cloned LDLR cDNA is sequenced to confirm its identity and to identify any additional sequence differences relative to wild-type. As one skilled in the art will recognize, this method can be used to identify variations in RNA splicing that are caused by LDLR mutation.

LDLR gene mutations may affect resistance to viral infection by modifying the properties of the resulting LDLR mRNA. Therefore, differences in mRNA stability between carriers of the LDLR alleles and homozygous wild-type subjects are evaluated. RNA stability is evaluated and compared using known assays including Taqman® and simple Northern hybridization. These constitute routine methods in molecular biology.

LDLR mutations may affect infection resistance by modifying the regulation of the LDLR gene. The mutant LDLR alleles may confer resistance to viral infection through constitutive expression, over-expression, under-expression, or other dysregulated expression. Several methods are used to evaluate gene expression. These methods include expression microarray analysis, Northern hybridization, Taqman®, and others. Samples are collected from tissues known to express the LDLR gene such as the peripheral blood mononuclear cells. Gene expression is compared between tissues from mutant LDLR carriers and non-carriers. In one embodiment, peripheral blood mononuclear cells are collected from carriers and non carriers, propagated in culture, and stimulated to express LDLR by treatment with lipoprotein deficient media. The level of expression of mutant LDLR alleles during induction is compared to wild-type alleles. In addition to evaluating LDLR gene expression by monitoring RNA levels, protein levels can also be evaluated using antibodies specific to the LDLR protein. As one skilled in the art will appreciate, numerous methods for evaluating LDLR protein levels exist including but not limited to western blotting, fluorescent microscopy, and fluorescent activated cell sorting. As one skilled in the art can appreciate, numerous combinations of tissues, experimental designs, and methods of analysis are used to evaluate mutant LDLR gene regulation.

LDLR mutations may affect infection resistance by modifying the normal splicing of the gene. As one skilled in the art will recognize, mutations in intronic sequences can result in the use of novel, alternate splice sites, inclusion of cryptic exons, the skipping of normal exons, or changes to the mRNA stability of mutant forms. Numerous methods can be used to evaluate changes in mRNA splicing in carriers of HCV resistance mutations, including in one preferred embodiment, the use of nested primers and reverse-transcriptase PCR to document and investigate all possible splice forms. As one skilled in the art will recognize, DNA sequencing can be used as an analytical compliment to any of these envisioned methods.

Once the mutated cDNA for each LDLR is cloned, it is used to manufacture recombinant LDLR proteins using any of a number of different known expression cloning systems. In one embodiment of this approach, a mutant LDLR cDNA is cloned by standard molecular biological methods into an Escherichia coli expression vector adjacent to an epitope tag that contains a sequence of DNA coding for a polyhistidine polypeptide. The recombinant protein is then purified from Escherichia coli lysates using immobilized metal affinity chromatography or similar method. One skilled in the art will recognize that there are many different expression vectors and host cells that can be used to purify recombinant proteins, including but not limited to yeast expression systems, baculovirus expression systems, Chinese hamster ovary cells, and others. As one skilled in the art will also appreciate, complex membrane bound proteins like LDLR, which are difficult to express in their entirety, can be studied through the expression of specific functional domains apart from the entire protein.

Computational methods are used to identify short peptide sequences from LDLR mutant proteins that uniquely distinguish these proteins from reference LDLR proteins. Various computational methods and commercially available software packages can be used for peptide selection. These computationally selected peptide sequences can be manufactured using the FMOC peptide synthesis chemistry or similar method. One skilled in the art will recognize that there are numerous chemical methods for synthesizing short polypeptides according to a supplied sequence.

Peptide fragments and the recombinant protein from the mutant or reference LDLR gene can be used to develop antibodies specific to this gene product. As one skilled in the art will recognize, there are numerous methods for antibody development involving the use of multiple different host organisms, adjuvants, etc. In one classic embodiment, a small amount (150 micrograms) of purified recombinant protein is injected subcutaneously into the backs of New Zealand White Rabbits with subsequent similar quantities injected every several months as boosters. Rabbit serum is then collected by venipuncture and the serum, purified IgG, or affinity purified antibody specific to the immunizing protein can be collected. As one skilled in the art will recognize, similar methods can be used to develop antibodies in rat, mouse, goat, and other organisms. Peptide fragments as described above can also be used to develop antibodies specific to the mutant LDLR protein. The development of both monoclonal and polyclonal antibodies is suitable for practicing the invention. The generation of mouse hybridoma cell lines secreting specific monoclonal antibodies to the mutant or reference LDLR proteins can be carried out by standard molecular techniques.

Antibodies prepared as described above can be used to develop diagnostic methods for evaluating the presence or absence of the mutant LDLR proteins in cells, tissues, and organisms. In one embodiment of this approach, antibodies specific to mutant LDLR proteins are used to detect these proteins in human cells and tissues by Western Blotting. These diagnostic methods can be used to validate the presence or absence of mutant LDLR proteins in the tissues of carriers and non-carriers of the above-described genetic mutations.

Antibodies prepared as described above can also be used to purify native mutant LDLR proteins from those patients who carry these mutations. Numerous methods are available for using antibodies to purify native proteins from human cells and tissues. In one embodiment, antibodies can be used in immunoprecipitation experiments involving homogenized human tissues and antibody capture using protein A. This method enables the concentration and further evaluation of mutant LDLR proteins. Numerous other methods for isolating the native forms of mutant LDLR are available including column chromatography, affinity chromatography, high pressure liquid chromatography, salting-out, dialysis, electrophoresis, isoelectric focusing, differential centrifugation, and others.

Proteomic methods are used to evaluate the effect of LDLR mutations on secondary, tertiary, and quaternary protein structure. Proteomic methods are also used to evaluate the impact of LDLR mutations on the post-translational modification of the LDLR protein. There are many known possible post-translational modifications to a protein including protease cleavage, glycosylation, phosphorylation, sulfation, the addition of chemical groups or complex molecules, and the like. A common method for evaluating secondary and tertiary protein structure is nuclear magnetic resonance (NMR) spectroscopy. NMR is used to probe differences in secondary and tertiary structure between wild-type LDLR proteins and mutant LDLR proteins. Modifications to traditional NMR are also suitable, including methods for evaluating the activity of functional sites including Transfer Nuclear Overhauser Spectroscopy (TrNOESY) and others. As one skilled in the art will recognize, numerous minor modifications to this approach and methods for data interpretation of results can be employed. All of these methods are intended to be included in practicing this invention. Other methods for determining protein structure by crystallization and X-ray diffraction are employed.

Mass spectroscopy can also be used to evaluate differences between mutant and wild-type LDLR proteins. This method can be used to evaluate structural differences as well as differences in the post-translational modifications of proteins. In one typical embodiment of this approach, the wild-type LDLR protein and mutant LDLR proteins are purified from human peripheral blood mononuclear cells using one of the methods described above. Purified proteins are digested with specific proteases (e.g. trypsin) and evaluated using mass spectrometry. As one skilled in the art will recognize, many alternative methods can also be used. This invention contemplates these additional alternative methods. For instance, either matrix-assisted laser desorption/ionization (MALDI) or electrospray ionization (ESI) mass spectrometric methods can be used. Furthermore, mass spectroscopy can be coupled with the use of two-dimensional gel electrophoretic separation of cellular proteins as an alternative to comprehensive pre-purification. Mass spectrometry can also be coupled with the use of peptide fingerprint database and various searching algorithms. Differences in post-translational modification, such as phosphorylation or glycosylation, can also be probed by coupling mass spectrometry with the use of various pretreatments such as with glycosylases and phosphatases. All of these methods are to be considered as part of this application.

LDLR may confer viral resistance by interaction with other proteins. According to the invention, LDLR-specific antibodies can be used to isolate protein complexes involving the LDLR proteins from a variety of sources as discussed above. As one skilled in the art will recognize, antibodies can be used with various cross-linking reagents to permit stabilization and enhanced purification of interacting protein complexes. These complexes can then be evaluated by gel electrophoresis to separate members of the interacting complex. Gels can be probed using numerous methods including Western blotting, and novel interacting proteins can be isolated and identified using peptide sequencing. Differences in the content of LDLR complexes in wild-type and mutant LDLR extracts will also be evaluated. As one skilled in the art will recognize, the described methods are only a few of numerous different approaches that can be used to purify, identify, and evaluate interacting proteins in the LDLR complex. Additional methods include, but are not limited to, phage display and the use of yeast two-hybrid methods.

LDLR is known to interact with hepatitis C virus particle and envelop E2 protein. Without being bound by a mechanism, the invention therefore relates to LDLR proteins that do not interact with the HCV virus particle or E2 protein, wherein the proteins are expressed by mRNA encoded by splice variants of LDLR, by LDLR polynucleotides having at least one mutation in the coding region, and or by LDLR polynucleotides having at least one base substitution, deletion or addition wherein binding to the HCV particle or E2 protein is altered or prevented.

Although the invention is not dependent on this model, the binding of E2 and/or the HCV particle to LDLR is consistent with a model in which mutated forms of LDLR avoid E2/HCV binding and HCV attachment is inhibited. In such cases, consistent with the clinical results described herein, a person carrying such a mutation is resistant to infection by hepatitis C virus. The mutation may in some cases directly affect the binding site of LDLR for E2/HCV. In other cases the mutation may be at a site separate from the actual binding site, but causes a conformational change such that binding of LDLR to E2/HCV is inhibited, slowed, or prevented.

The binding of LDLR to E2/HCV in a physiologically and pathologically relevant manner therefore provides an objective test for assaying the effect of a base mutation, deletion or addition in an LDLR polynucleotide.

LDLR proteins are receptors that normally function by binding to and endocytosing a variety of ligands including apolipoprotein B and apolipoprotein E-containing cholesterol complexes. The effects of mutations in LDLR on the ability of this receptor to carry out these normal functions of ligand binding and endocytosis can be evaluated. As one skilled in the art will appreciate, numerous methods can be used to evaluate ligand binding, receptor mediated endocytosis, endosomal ligand release, and receptor recycling. All of these methods are to be considered part of this application. In one preferred embodiment, the binding and internalization of radiolabelled or fluorescently labeled ligands can be evaluated in cells expressing normal and mutant forms of LDLR. In another preferred embodiment, antibodies directed to LDL-cholesterol and LDLR can be used in double colloidal gold immuno-electron microscopy in order to monitor the degree and efficiency of receptor internalization, ligand release, and ligand recycling for normal and mutant forms of LDLR. In a still further embodiment, assays can be developed that measure both the antiviral activity of therapeutic compounds as well as their ability to modulate LDL and VLDL binding to LDLR, receptor mediated endocytosis and receptor recycling. As one skilled in the art will recognize, preferred therapeutic compounds will inhibit the binding and internalization of virus particles without inhibiting the normal function of LDLR in cholesterol uptake and metabolism.

Biological studies are performed to evaluate the degree to which LDLR mutant genes protect from viral infection. These biological studies generally take the form of introducing the mutant LDLR genes or proteins into cells or whole organisms, and evaluating their biological and antiviral activities relative to wild-type controls. In one typical embodiment of this approach, the mutant LDLR genes are introduced into African Green monkey kidney (Vero) cells in culture by cloning the cDNAs isolated as described herein into a mammalian expression vector that drives expression of the cloned cDNA from an SV40 promoter sequence. This vector will also contain SV40 and cytomegalovirus enhancer elements that permit efficient expression of the mutant LDLR genes, and a neomycin resistance gene for selection in culture. The biological effects of mutant LDLR expression can then be evaluated in Vero cells infected with the dengue virus. In the event that mutant LDLR confers broad resistance to multiple flaviviruses, one would expect an attenuation of viral propagation in cell lines expressing these mutant forms of LDLR relative to wild-type. As one skilled in the art will recognize, there are multiple different experimental approaches that can be used to evaluate the biological effects of mutant LDLR genes and proteins in cells and organisms and in response to different infectious agents. For instance, in the above example, different expression vectors, cell types, and viral species may be used to evaluate the mutant LDLR resistance effects. Primary human cells in culture may be evaluated as opposed to cell lines. Cell lines deficient for expression of normal LDLR may be used. Expression vectors containing alternative promoter and enhancer sequences may be evaluated. Viruses other than the flaviviruses (e.g. respiratory syncytial virus and picornavirus) are also evaluated.

Transgenic animal models are developed to assess the usefulness of mutant forms of LDLR in protecting against whole-organism viral infection. In one embodiment, LDLR genes are introduced into the genomes of mice susceptible to flavivirus infection (e.g. the C3H/He inbred laboratory strain). Positive-negative selection-based methods can be used to knock-out the native LDLR gene in mice with the transgene in order to assess LDLR mutant function in the absence of wild-type protein. These mutant LDLR genes are evaluated for their ability to modify infection or confer resistance to infection in susceptible mice. As one skilled in the art will appreciate, numerous standard methods can be used to introduce transgenic human mutant LDLR genes into mice. These methods can be combined with other methods that affect tissue specific expression patterns or that permit regulation of the transgene through the introduction of endogenous chemicals, the use of inducible or tissue specific promoters, etc.

As a model for hepatitis C infection, cell lines expressing mutant LDLR genes can be evaluated for susceptibility, resistance, or modification of infection with the bovine diarrheal virus (BVDV) or the GB virus C (GBV-C). BVDV and GBV-C are commonly used models for testing the efficacy of potential anti-HCV antiviral drugs. In one embodiment, the mutant LDLR genes can be introduced into KL (calf lung) cells using expression vectors essentially as described above and tested for their ability to modify BVDV infection in this cell line. Furthermore, mouse models of HCV infection (e.g. the transplantation of human livers into mice, the infusion of human hepatocyte into mouse liver, etc.) may also be evaluated for modification of HCV infection in the transgenic setting of mutant LDLR genes. Experiments can be performed whereby the effects of expression of mutant LDLR genes are assessed in HCV viral culture and replicon systems. As one skilled in the art will appreciate, other viral models may be used, as for example the GB virus B. Furthermore, the ability of defective interfering viruses to potentiate the effects of mutant LDLR forms can be tested in cell culture and in small animal models.

The degree to which the presence or absence of mutant LDLR genotypes affects other human phenotypes can also be examined. For instance, LDLR mutations are evaluated for their association with viral titer and spontaneous viral clearance in HCV infected subjects. Similar methods of correlating host LDLR genotype with the course of other virus or flavivirus infections can also be undertaken. The impact of LDLR mutations on promoting successful outcomes during interferon or interferon with ribavirin treatment in HCV infected patients is also examined. These mutations may not only confer a level of infection resistance, but also promote spontaneous viral clearance in infected subjects with or without interferon-ribavirin treatment. Furthermore, it has been reported that schizophrenia occurs at a higher frequency in geographic areas that are endemic for flavivirus infection, suggesting an association between flavivirus resistance alleles and predisposition to schizophrenia. This link is evaluated by performing additional genetic association studies involving the schizophrenia phenotype and the LDLR mutations. As one skilled in the art will recognize, LDLR mutations may affect neurological function by modulating clearance of apolipoprotein E containing molecules.

Polynucleotide Analysis

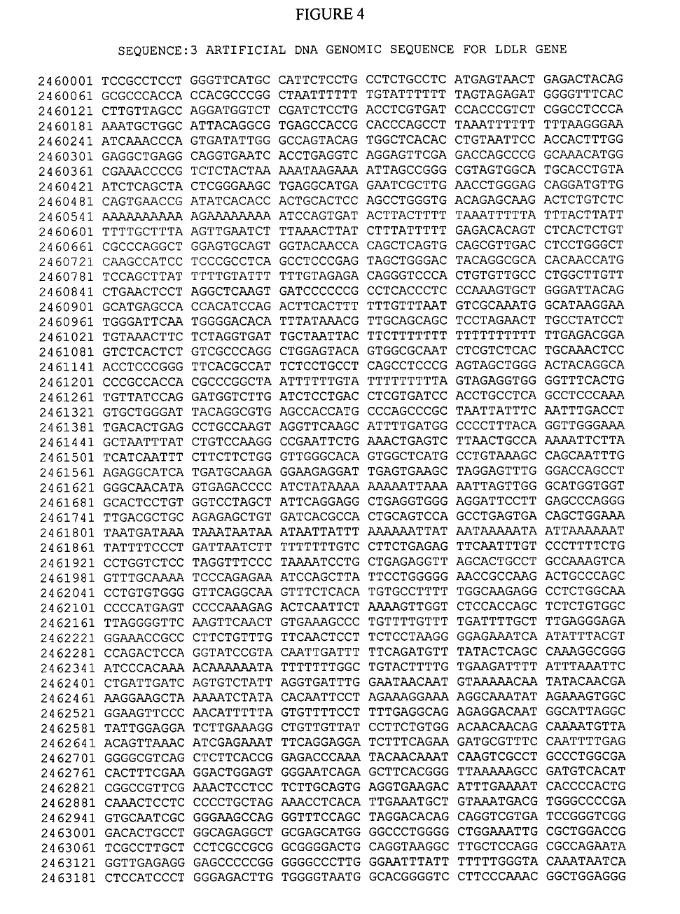

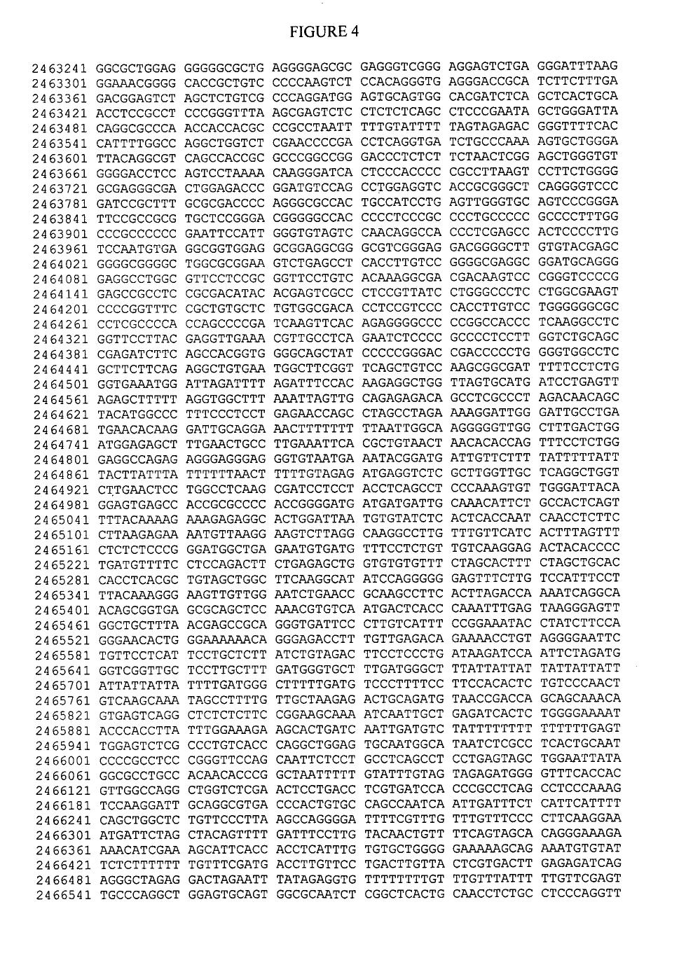

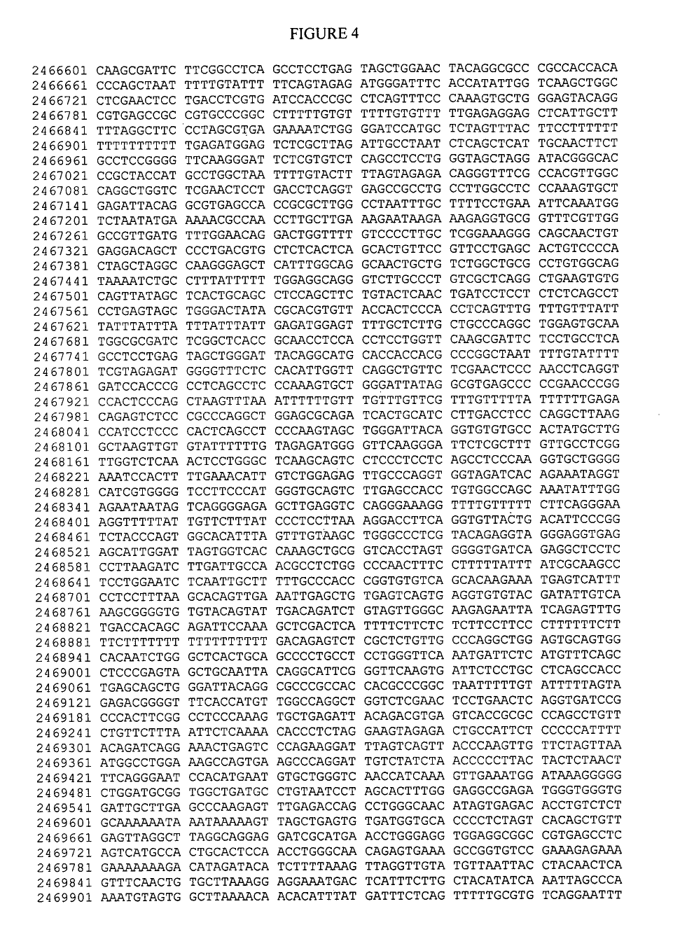

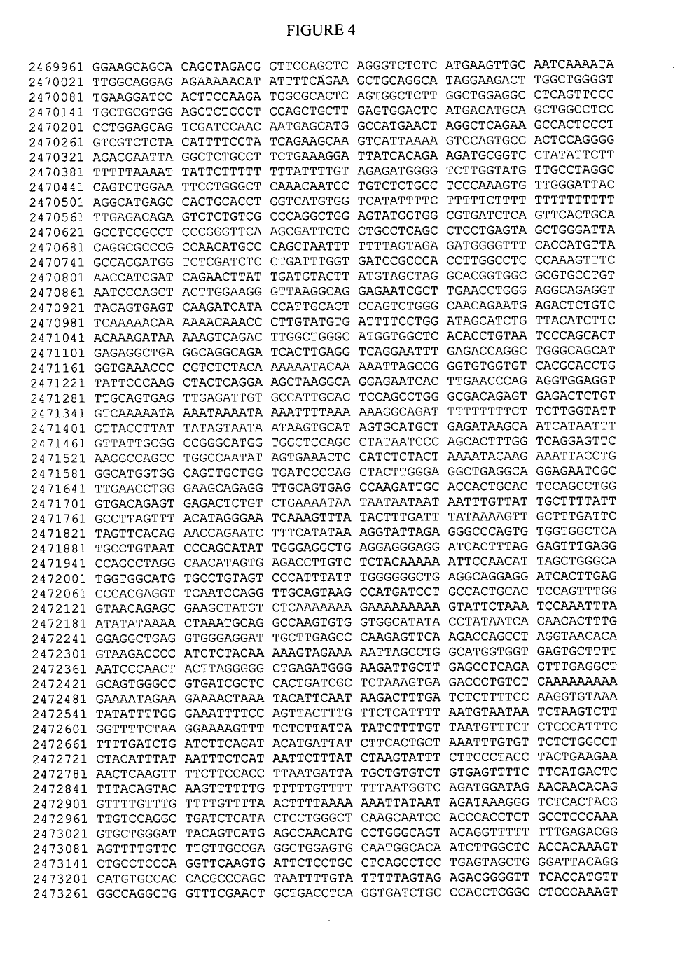

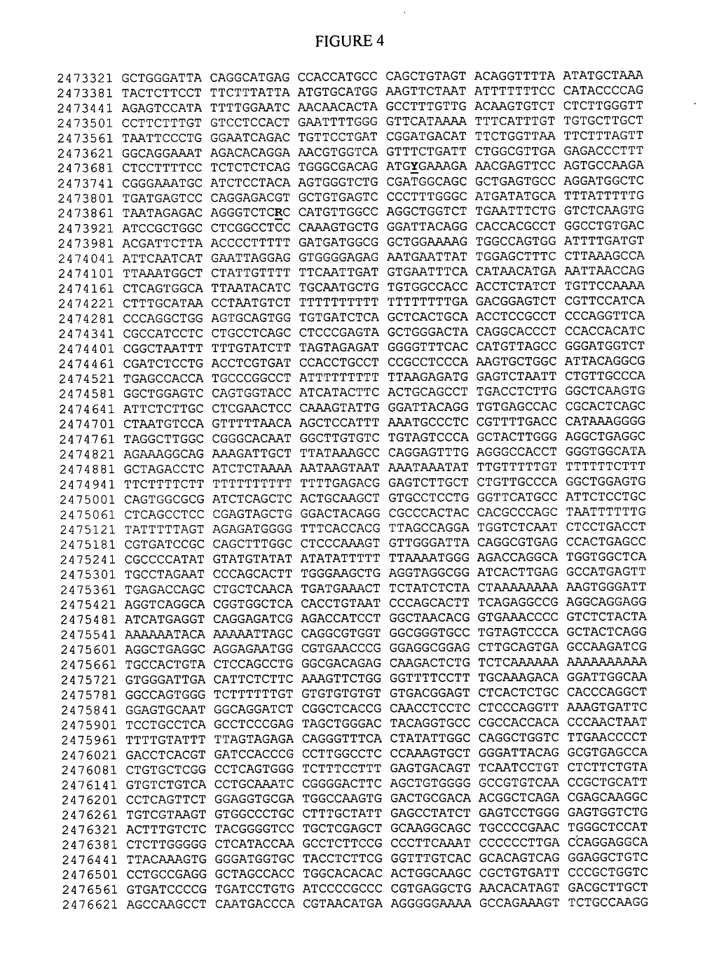

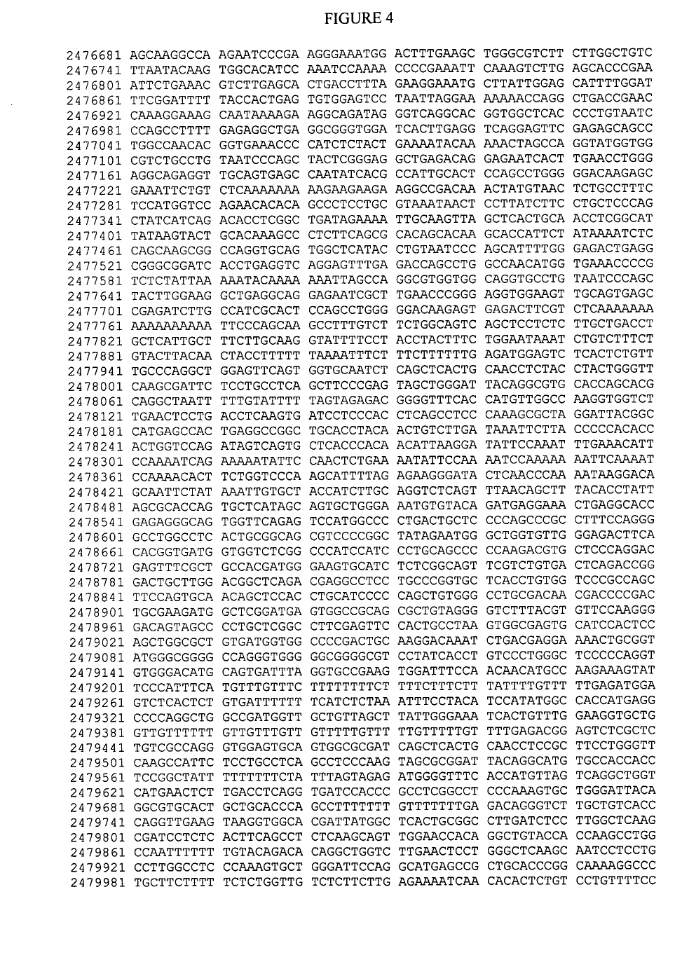

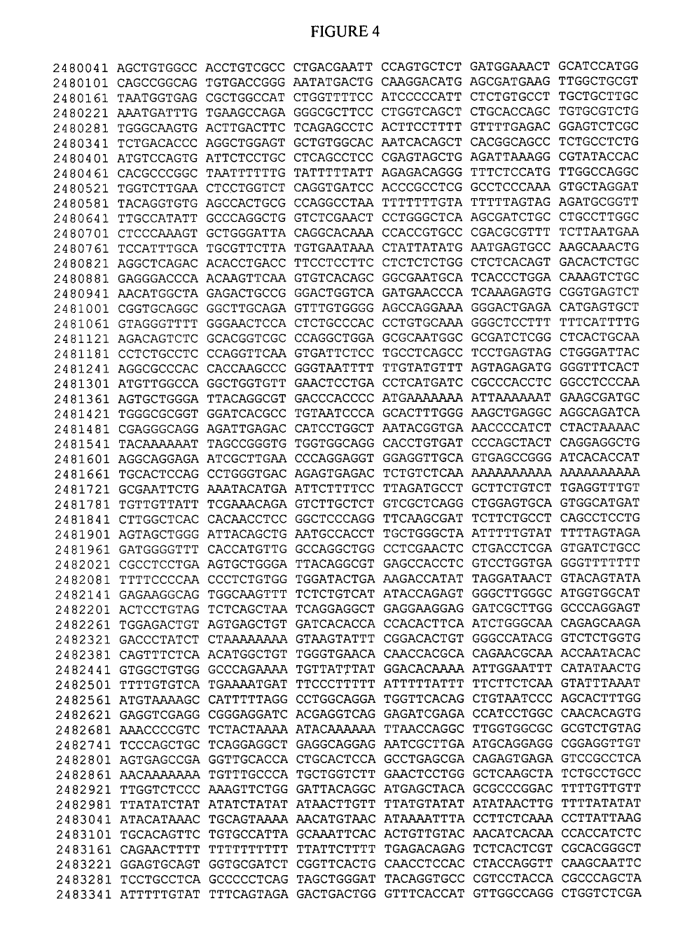

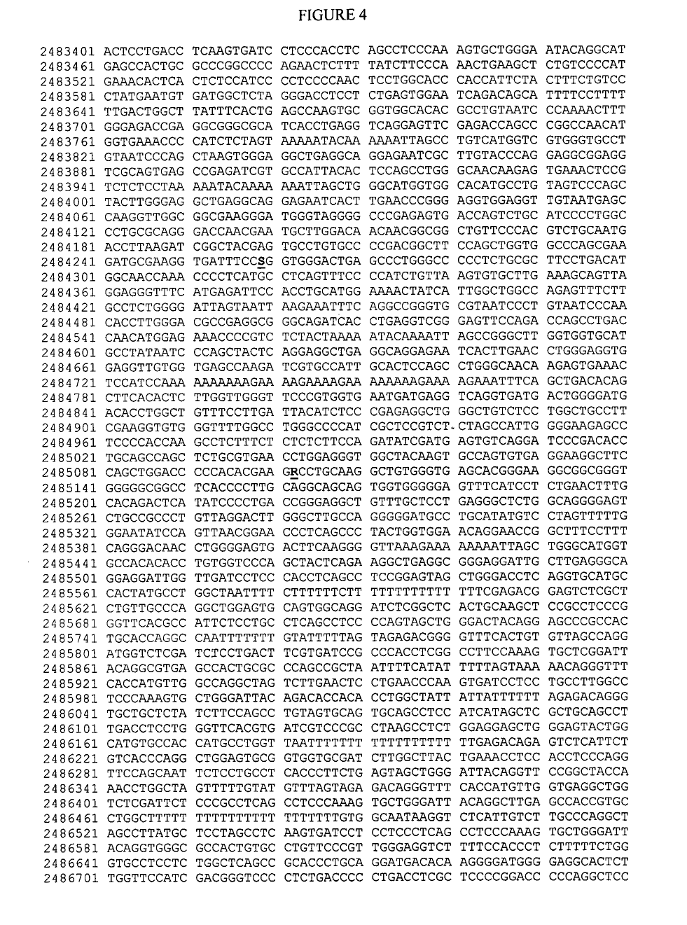

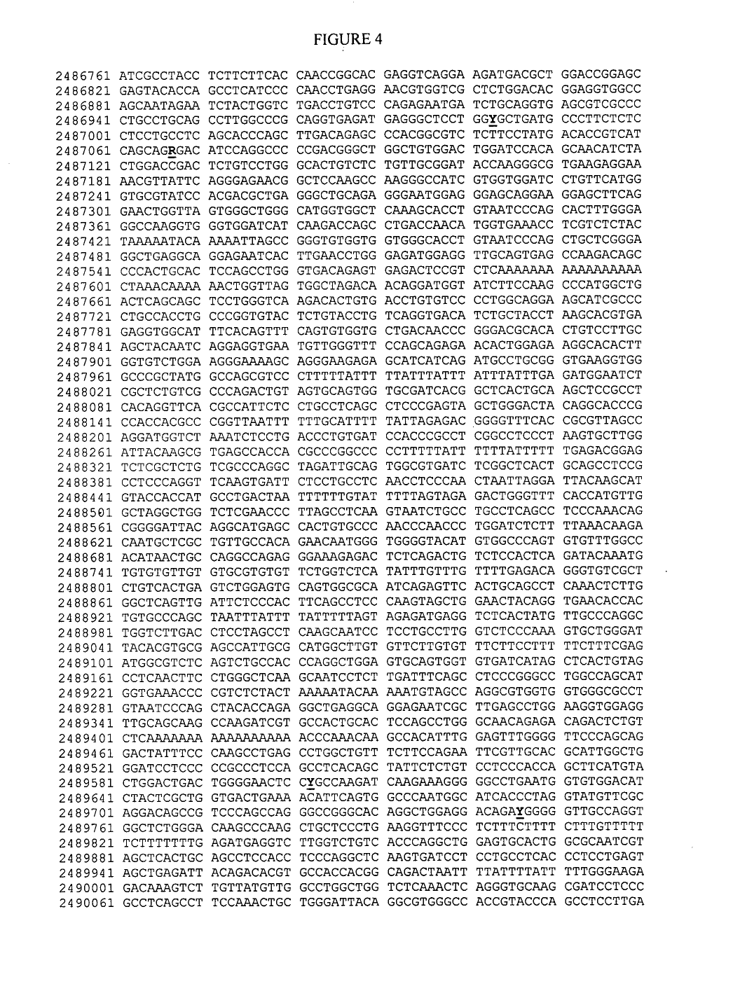

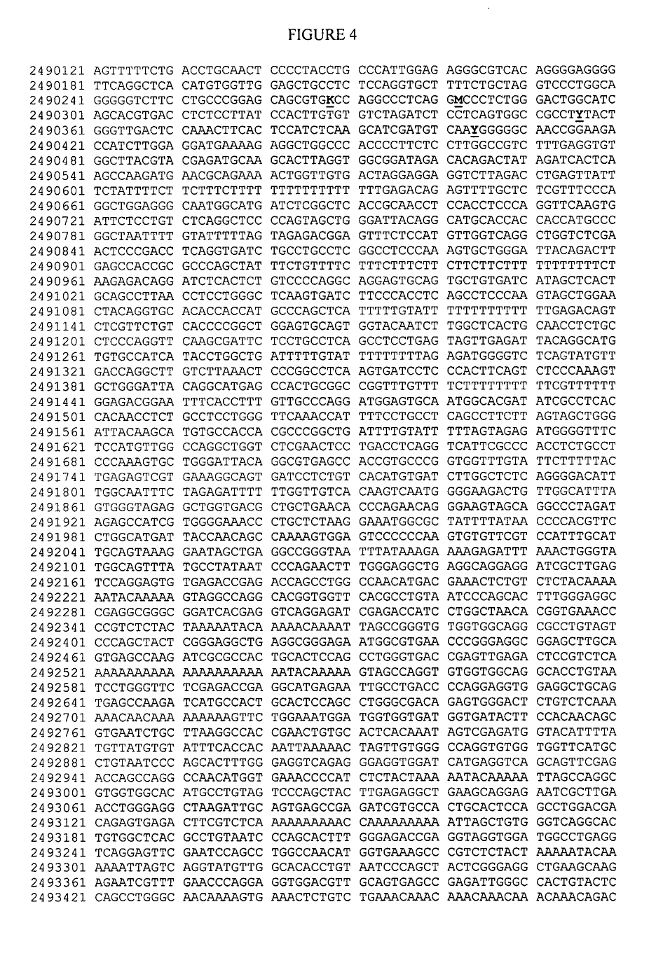

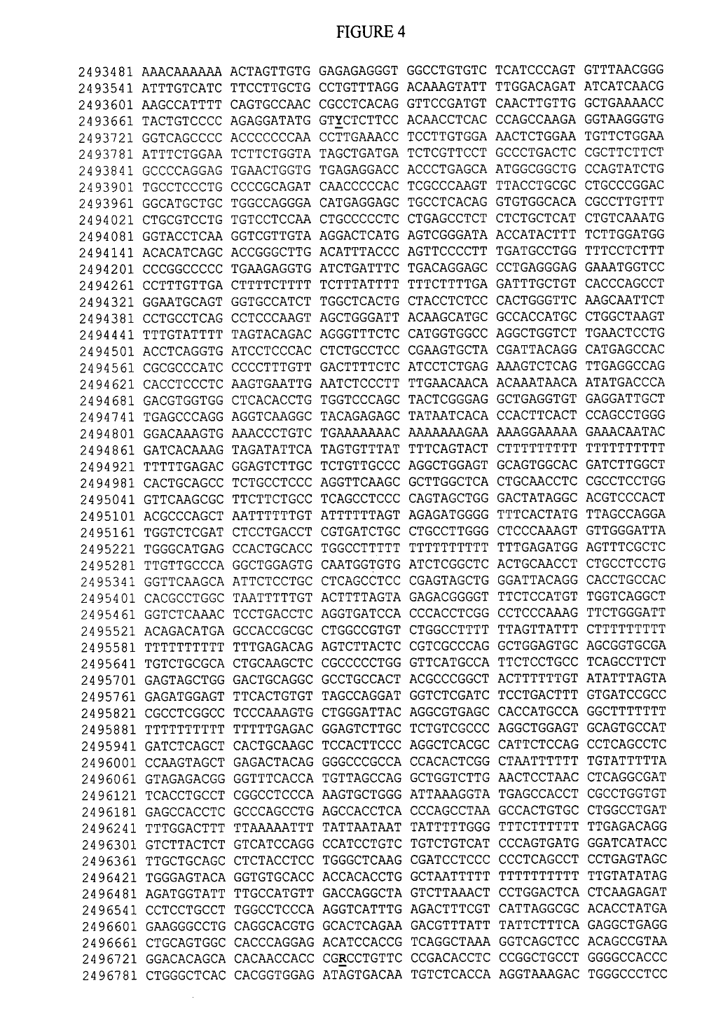

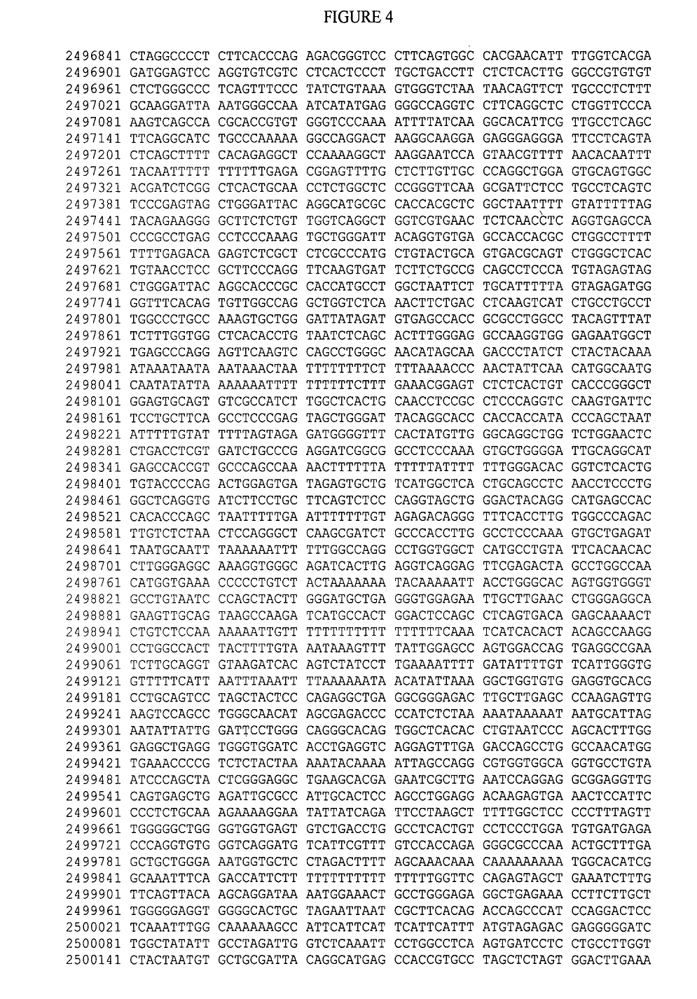

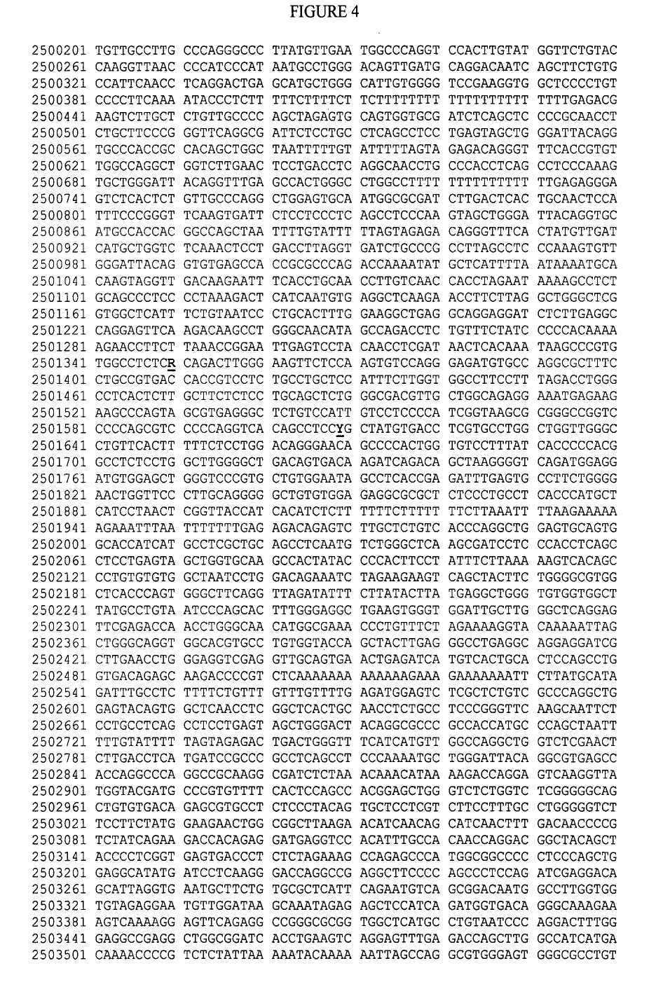

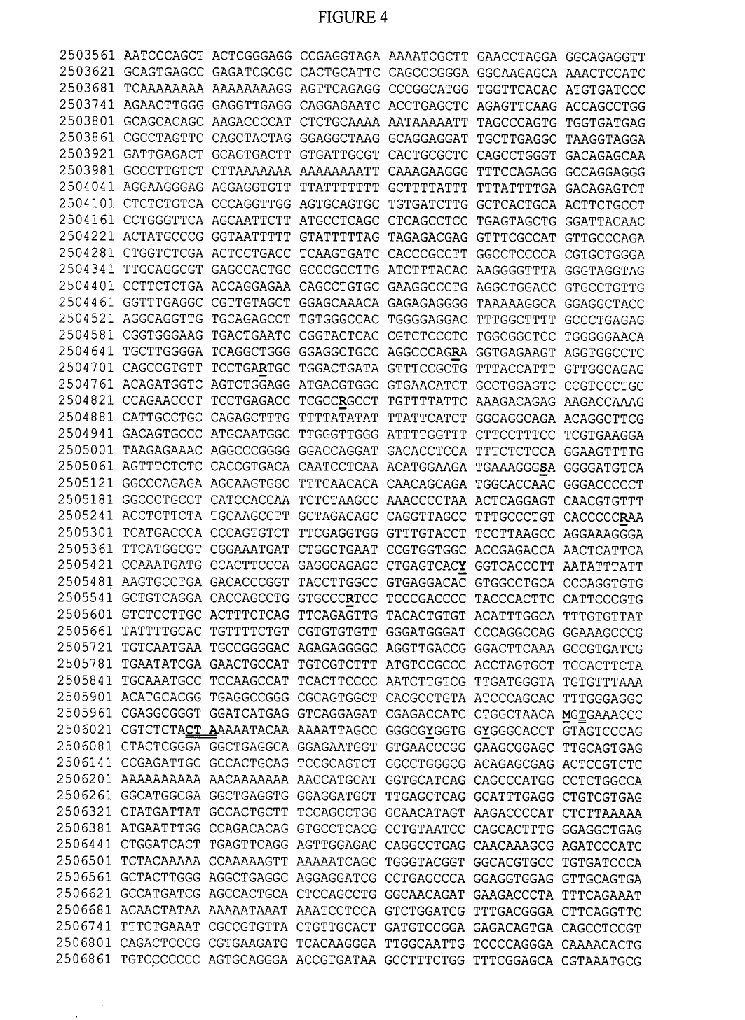

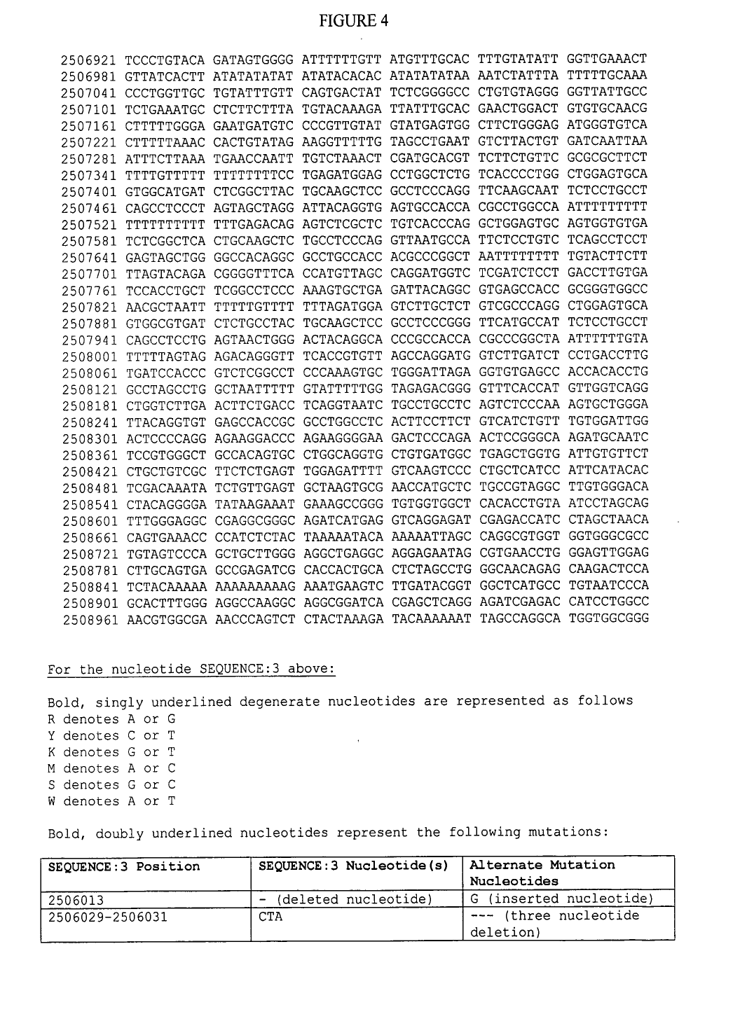

The low density lipoprotein receptor gene is a nucleic acid whose nucleotide sequence codes for low density lipoprotein receptor, mutant low density lipoprotein receptor, or low density lipoprotein receptor pseudogene. It can be in the form of genomic DNA, an mRNA or cDNA, and in single or double stranded form. Preferably, genomic DNA is used because of its relative stability in biological samples compared to mRNA. The sequence of a polynucleotide consisting of consecutive nucleotides 2,460,001-2,509,020 of the complete genomic sequence of the reference low density lipoprotein receptor gene is provided in FIG. 1 as SEQUENCE:1, and corresponds to Genbank Accession No. NT_{—}011295.10. An artificial sequence of a portion of the LDLR gene showing the mutations disclosed herein is provided in SEQUENCE:3 of FIG. 4. For the nucleotide SEQUENCE:3, bold, singly underlined degenerate nucleotides are represented as follows: R denotes A or G; Y denotes C or T; K denotes G or T; M denotes A or C; S denotes G or C; and W denotes A or T. Bold, doubly underlined nucleotides represent the following mutations:

| SEQUENCE: 3 | SEQUENCE: 3 | Alternate Mutation |
|---|---|---|
| Position | Nucleotide(s) | Nucleotides |
| 2506013 | - (deleted | G (inserted nucleotide) |
| | nucleotide) | |
| 2506029-2506031 | CTA | --- (three nucleotide deletion) |

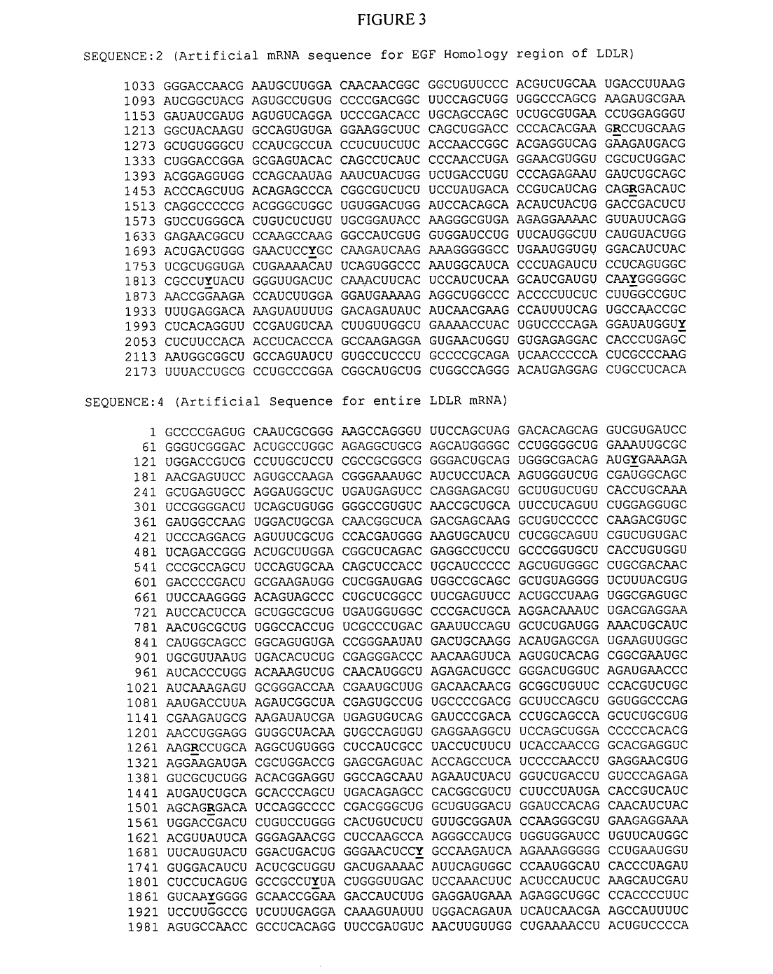

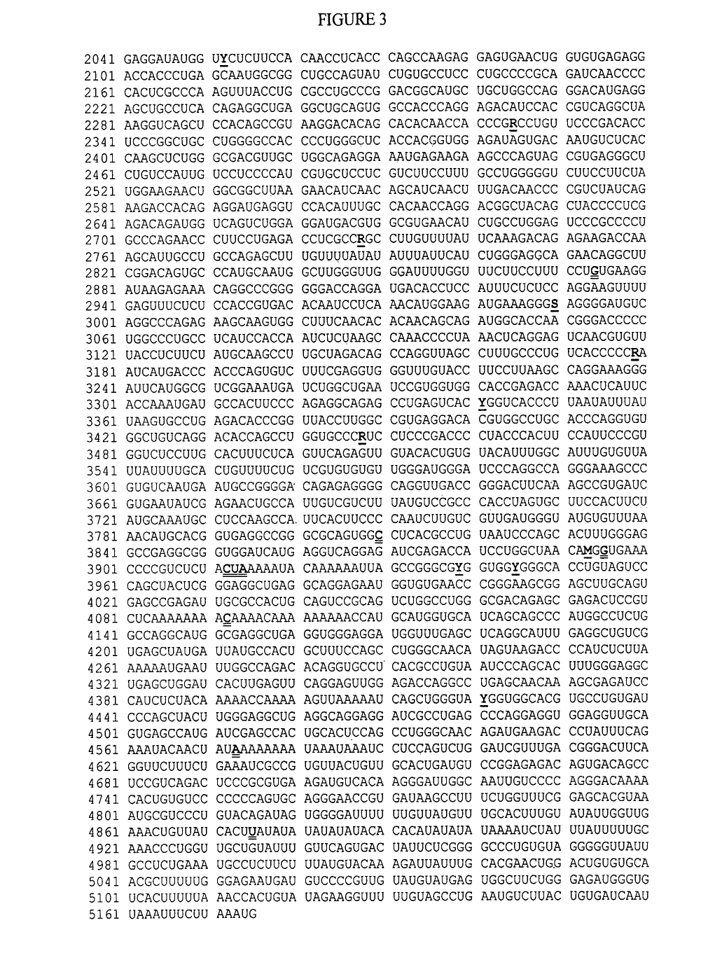

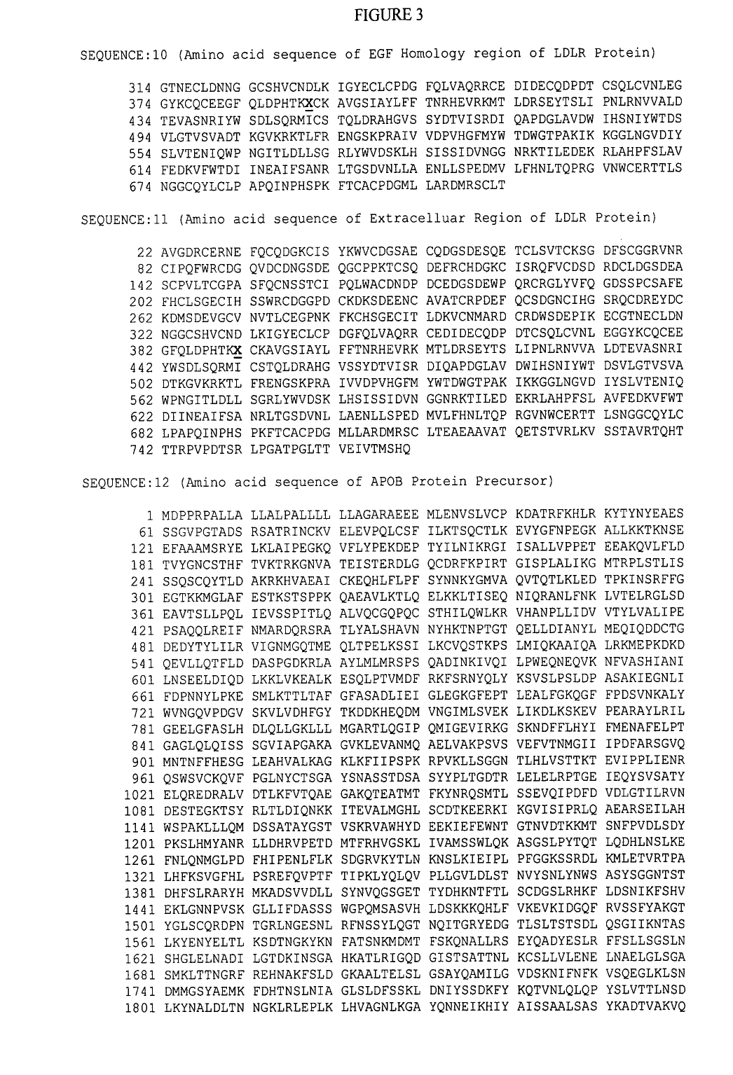

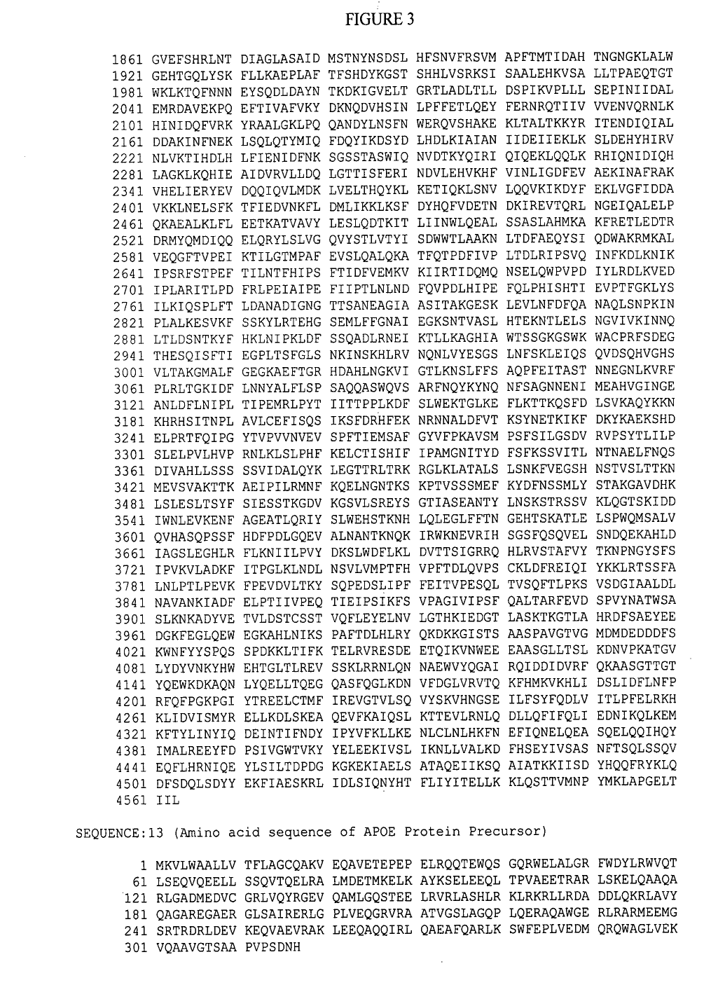

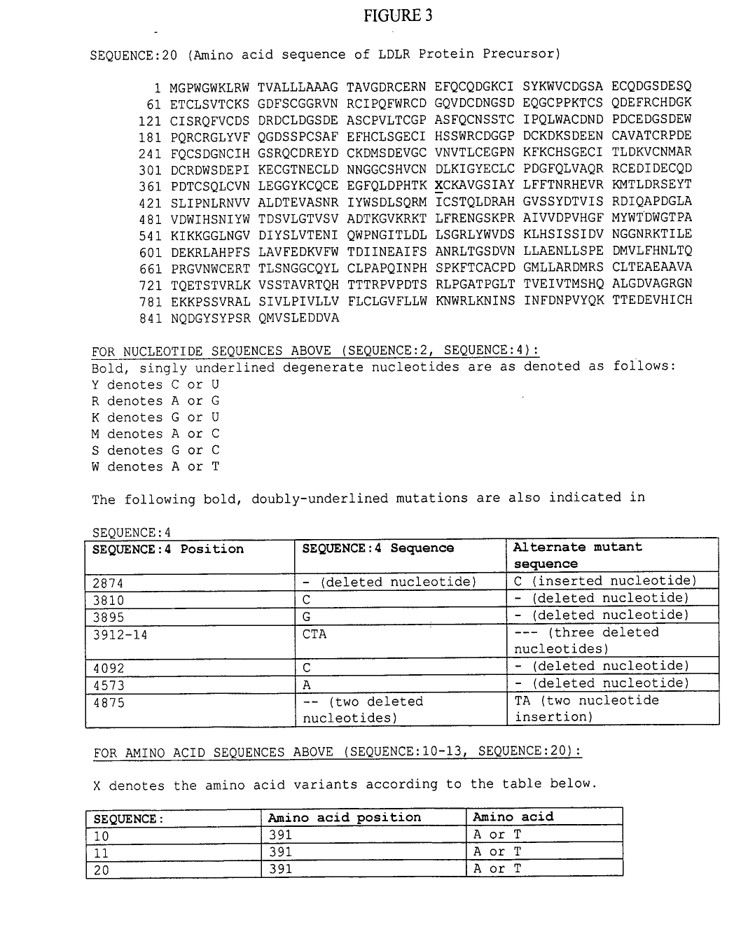

An artificial sequence of the mRNA sequence of the LDLR gene showing the mutations disclosed herein is provided in SEQUENCE:4 of FIG. 3. An artificial mRNA sequence of the EGF precursor homology domain of LDLR showing the mutations disclosed herein is provided in SEQUENCE:2 of FIG. 3. For nucleotide sequences SEQUENCE:2 and SEQUENCE:4, bold, singly underlined degenerate nucleotides are as denoted as follows: Y denotes C or U; R denotes A or G; K denotes G or U; M denotes A or C; S denotes G or C; and W denotes A or T.

The following bold, doubly-underlined mutations are also indicated in SEQUENCE:4

| SEQUENCE: 4 | SEQUENCE: 4 | |
|---|---|---|
| Position | Sequence | Alternate mutant sequence |
| 2874 | - (deleted | C (inserted nucleotide) |
| | nucleotide) | |
| 3810 | C | - (deleted nucleotide) |
| 3895 | G | - (deleted nucleotide) |
| 3912-14 | CTA | --- (three deleted nucleotides) |
| 4092 | C | - (deleted nucleotide) |
| 4573 | A | - (deleted nucleotide) |
| 4875 | -- (two deleted | TA (two nucleotide insertion) |
| | nucleotides) | |

For amino acid sequences SEQUENCE:10-13, and SEQUENCE:20, X denotes the amino acid variants according to the table below.

| SEQUENCE: | Amino acid position | Amino acid |
|---|---|---|
| 10 | 391 | A or T |
| 11 | 391 | A or T |
| 20 | 391 | A or T |

The nucleic acid sample is obtained from cells, typically peripheral blood leukocytes. Where mRNA is used, the cells are lysed under RNase inhibiting conditions. In one embodiment, the first step is to isolate the total cellular mRNA. Poly A+ mRNA can then be selected by hybridization to an oligo-dT cellulose column.

In preferred embodiments, the nucleic acid sample is enriched for a presence of low density lipoprotein receptor allelic material. Enrichment is typically accomplished by subjecting the genomic DNA or mRNA to a primer extension reaction employing a polynucleotide synthesis primer as described herein. Particularly preferred methods for producing a sample to be assayed use preselected polynucleotides as primers in a polymerase chain reaction (PCR) to form an amplified (PCR) product.

Preparation of Polynucleotide Primers

The term “polynucleotide” as used herein in reference to primers, probes and nucleic acid fragments or segments to be synthesized by primer extension is defined as a molecule comprised of two or more deoxyribonucleotides or ribonucleotides, preferably more than three. Its exact size will depend on many factors, which in turn depends on the ultimate conditions of use.

The term “primer” as used herein refers to a polynucleotide whether purified from a nucleic acid restriction digest or produced synthetically, which is capable of acting as a point of initiation of nucleic acid synthesis when placed under conditions in which synthesis of a primer extension product which is complementary to a nucleic acid strand is induced, i.e., in the presence of nucleotides and an agent for polymerization such as DNA polymerase, reverse transcriptase and the like, and at a suitable temperature and pH. The primer is preferably single stranded for maximum efficiency, but may alternatively be in double stranded form. If double stranded, the primer is first treated to separate it from its complementary strand before being used to prepare extension products. Preferably, the primer is a polydeoxyribonucleotide. The primer must be sufficiently long to prime the synthesis of extension products in the presence of the agents for polymerization. The exact lengths of the primers will depend on many factors, including temperature and the source of primer. For example, depending on the complexity of the target sequence, a polynucleotide primer typically contains 15 to 25 or more nucleotides, although it can contain fewer nucleotides. Short primer molecules generally require cooler temperatures to form sufficiently stable hybrid complexes with template.

The primers used herein are selected to be “substantially” complementary to the different strands of each specific sequence to be synthesized or amplified. This means that the primer must be sufficiently complementary to non-randomly hybridize with its respective template strand. Therefore, the primer sequence may or may not reflect the exact sequence of the template. For example, a non-complementary nucleotide fragment can be attached to the 5′ end of the primer, with the remainder of the primer sequence being substantially complementary to the strand. Such non-complementary fragments typically code for an endonuclease restriction site. Alternatively, non-complementary bases or longer sequences can be interspersed into the primer, provided the primer sequence has sufficient complementarity with the sequence of the strand to be synthesized or amplified to non-randomly hybridize therewith and thereby form an extension product under polynucleotide synthesizing conditions.

Primers of the present invention may also contain a DNA-dependent RNA polymerase promoter sequence or its complement. See for example, Krieg, et al., Nucl. Acids Res., 12:7057-70 (1984); Studier, et al., J. Mol. Biol., 189:113-130 (1986); and Molecular Cloning: A Laboratory Manual, Second Edition, Maniatis, et al., eds., Cold Spring Harbor, N.Y. (1989).

When a primer containing a DNA-dependent RNA polymerase promoter is used, the primer is hybridized to the polynucleotide strand to be amplified and the second polynucleotide strand of the DNA-dependent RNA polymerase promoter is completed using an inducing agent such as E. coli DNA polymerase I, or the Klenow fragment of E. coli DNA polymerase. The starting polynucleotide is amplified by alternating between the production of an RNA polynucleotide and DNA polynucleotide.

Primers may also contain a template sequence or replication initiation site for a RNA-directed RNA polymerase. Typical RNA-directed RNA polymerases include the QB replicase described by Lizardi, et al., Biotechnology, 6:1197-1202 1988). RNA-directed polymerases produce large numbers of RNA strands from a small number of template RNA strands that contain a template sequence or replication initiation site. These polymerases typically give a one million-fold amplification of the template strand as has been described by Kramer, et al., J. Mol. Biol., 89:719-736 (1974).

The polynucleotide primers can be prepared using any suitable method, such as, for example, the phosphotriester or phosphodiester methods, see Narang, et al., Meth. Enzymol., 68:90, (1979); U.S. Pat. Nos. 4,356,270, 4,458,066, 4,416,988, 4,293,652; and Brown, et al., Meth. Enzymol., 68:109 (1979).

The choice of a primer's nucleotide sequence depends on factors such as the distance on the nucleic acid from the hybridization point to the region coding for the mutation to be detected, its hybridization site on the nucleic acid relative to any second primer to be used, and the like.

If the nucleic acid sample is to be enriched for low density lipoprotein receptor gene material by PCR amplification, two primers, i.e., a PCR primer pair, must be used for each coding strand of nucleic acid to be amplified. The first primer becomes part of the non-coding (anti-sense or minus or complementary) strand and hybridizes to a nucleotide sequence on the plus or coding strand. Second primers become part of the coding (sense or plus) strand and hybridize to a nucleotide sequence on the minus or non-coding strand. One or both of the first and second primers can contain a nucleotide sequence defining an endonuclease recognition site. The site can be heterologous to the low density lipoprotein receptor gene being amplified.

In one embodiment, the present invention utilizes a set of polynucleotides that form primers having a priming region located at the 3′-terminus of the primer. The priming region is typically the 3′-most (3′-terminal) 15 to 30 nucleotide bases. The 3′-terminal priming portion of each primer is capable of acting as a primer to catalyze nucleic acid synthesis, i.e., initiate a primer extension reaction off its 3′ terminus. One or both of the primers can additionally contain a 5′-terminal (5′-most) non-priming portion, i.e., a region that does not participate in hybridization to the preferred template.

In PCR, each primer works in combination with a second primer to amplify a target nucleic acid sequence. The choice of PCR primer pairs for use in PCR is governed by considerations as discussed herein for producing low density lipoprotein receptor gene regions. When a primer sequence is chosen to hybridize (anneal) to a target sequence within the low density lipoprotein receptor gene allele intron, the target sequence should be conserved among the alleles in order to insure generation of target sequence to be assayed.

Polymerase Chain Reaction

Low density lipoprotein receptor genes are comprised of polynucleotide coding strands, such as mRNA and/or the sense strand of genomic DNA. If the genetic material to be assayed is in the form of double stranded genomic DNA, it is usually first denatured, typically by melting, into single strands. The nucleic acid is subjected to a PCR reaction by treating (contacting) the sample with a PCR primer pair, each member of the pair having a preselected nucleotide sequence. The PCR primer pair is capable of initiating primer extension reactions by hybridizing to nucleotide sequences, preferably at least about 10 nucleotides in length, more preferably at least about 20 nucleotides in length, conserved within the low density lipoprotein receptor alleles. The first primer of a PCR primer pair is sometimes referred to herein as the “anti-sense primer” because it hybridizes to a non-coding or anti-sense strand of a nucleic acid, i.e., a strand complementary to a coding strand. The second primer of a PCR primer pair is sometimes referred to herein as the “sense primer” because it hybridizes to the coding or sense strand of a nucleic acid.

The PCR reaction is performed by mixing the PCR primer pair, preferably a predetermined amount thereof, with the nucleic acids of the sample, preferably a predetermined amount thereof, in a PCR buffer to form a PCR reaction admixture. The admixture is thermocycled for a number of cycles, which is typically predetermined, sufficient for the formation of a PCR reaction product, thereby enriching the sample to be assayed for low density lipoprotein receptor genetic material.

PCR is typically carried out by thermocycling i.e., repeatedly increasing and decreasing the temperature of a PCR reaction admixture within a temperature range whose lower limit is about 30 degrees Celsius (30° C.) to about 55° C. and whose upper limit is about 90° C. to about 100° C. The increasing and decreasing can be continuous, but is preferably phasic with time periods of relative temperature stability at each of temperatures favoring polynucleotide synthesis, denaturation and hybridization.

A plurality of first primer and/or a plurality of second primers can be used in each amplification, e.g., one species of first primer can be paired with a number of different second primers to form several different primer pairs. Alternatively, an individual pair of first and second primers can be used. In any case, the amplification products of amplifications using the same or different combinations of first and second primers can be combined for assaying for mutations.

The PCR reaction is performed using any suitable method. Generally it occurs in a buffered aqueous solution, i.e., a PCR buffer, preferably at a pH of 7-9, most preferably about 8. Preferably, a molar excess (for genomic nucleic acid, usually about 10⁶:1 primer:template) of the primer is admixed to the buffer containing the template strand. A large molar excess is preferred to improve the efficiency of the process.

The PCR buffer also contains the deoxyribonucleotide triphosphates (polynucleotide synthesis substrates) dATP, dCTP, dGTP, and dTTP and a polymerase, typically thermostable, all in adequate amounts for primer extension (polynucleotide synthesis) reaction. The resulting solution (PCR admixture) is heated to about 90° C.-100° C. for about 1 to 10 minutes, preferably from 1 to 4 minutes. After this heating period the solution is allowed to cool to 54° C., which is preferable for primer hybridization. The synthesis reaction may occur at from room temperature up to a temperature above which the polymerase (inducing agent) no longer functions efficiently. The thermocycling is repeated until the desired amount of PCR product is produced. An exemplary PCR buffer comprises the following: 50 mM KCl; 10 mM Tris-HCl at pH 8.3; 1.5 mM MgCl; 0.001% (wt/vol) gelatin, 200 μM dATP; 200 μM dTTP; 200 μM dCTP; 2002 μM dGTP; and 2.5 units Thermus aquaticus (Taq) DNA polymerase I (U.S. Pat. No. 4,889,818) per 100 microliters of buffer.

The inducing agent may be any compound or system which will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, reverse transcriptase, and other enzymes, including heat-stable enzymes, which will facilitate combination of the nucleotides in the proper manner to form the primer extension products which are complementary to each nucleic acid strand. Generally, the synthesis will be initiated at the 3′ end of each primer and proceed in the 5′ direction along the template strand, until synthesis terminates, producing molecules of different lengths. There may be inducing agents, however, which initiate synthesis at the 5′ end and proceed in the above direction, using the same process as described above.

The inducing agent also may be a compound or system which will function to accomplish the synthesis of RNA primer extension products, including enzymes. In preferred embodiments, the inducing agent may be a DNA-dependent RNA polymerase such as T7 RNA polymerase, T3 RNA polymerase or SP6 RNA polymerase. These polymerases produce a complementary RNA polynucleotide. The high turn-over rate of the RNA polymerase amplifies the starting polynucleotide as has been described by Chamberlin, et al., The Enzymes, ed. P. Boyer, pp. 87-108, Academic Press, New York (1982). Amplification systems based on transcription have been described by Gingeras, et al., in PCR Protocols, A Guide to Methods and Applications, pp. 245-252, Innis, et al., eds, Academic Press, Inc., San Diego, Calif. (1990).

If the inducing agent is a DNA-dependent RNA polymerase and, therefore incorporates ribonucleotide triphosphates, sufficient amounts of ATP, CTP, GTP and UTP are admixed to the primer extension reaction admixture and the resulting solution is treated as described above.

The newly synthesized strand and its complementary nucleic acid strand form a double-stranded molecule which can be used in the succeeding steps of the process.

The PCR reaction can advantageously be used to incorporate into the product a preselected restriction site useful in detecting a mutation in the low density lipoprotein receptor gene.

PCR amplification methods are described in detail in U.S. Pat. Nos. 4,683,192, 4,683,202, 4,800,159, and 4,965,188, and at least in several texts including PCR Technology: Principles and Applications for DNA Amplification, H. Erlich, ed., Stockton Press, New York (1989); and PCR Protocols: A Guide to Methods and Applications, Innis, et al., eds., Academic Press, San Diego, Calif. (1990).

In some embodiments, two pairs of first and second primers are used per amplification reaction. The amplification reaction products obtained from a plurality of different amplifications, each using a plurality of different primer pairs, can be combined or assayed separately.

However, the present invention contemplates amplification using only one pair of first and second primers. Exemplary primers for amplifying the sections of DNA containing the mutations disclosed herein are shown below in Table 1. Table 2 shows the position of each mutation of the present invention within its respective containing Amplicon.

TABLE 1

| | | | Product size |
|---|---|---|---|
| Amplicon | PrimerA | PrimerB | (bp) |
| Amplicon2 | 5′- ATTCCCTGGGAATCAGACTG -3′ | 5′- TAAGAATCGTGTCACAGGCC -3′ | 428 |
| | (SEQUENCE:21) | (SEQUENCE:22) | |
| Amplicon7 | 5′- GGCAGGAGAATCACTTGAAC -3′ | 5′- TTCCATGCAGGTGGAATCTC -3′ | 377 |
| | (SEQUENCE:23) | (SEQUENCE:24) | |
| Amplicon8 | 5′- ATTACATCTCCCGAGAGGCT -3′ | 5′- GTTCAGAGGATGAAACTCCC -3′ | 337 |
| | (SEQUENCE:25) | (SEQUENCE:26) | |
| Amplicon23 | 5′- GGGGAGGCACTCTTGGTT -3′ | 5′- GCTCCCTCCATTCCCTCT -3′ | 598 |
| | (SEQUENCE:27) | (SEQUENCE:28) | |
| Amplicon10 | 5′- GCAGGACTATTTCCCAAGCC -3′ | 5′- TGAGCTACGATTGCGCCAGT -3′ | 430 |
| | (SEQUENCE:29) | (SEQUENCE:30) | |
| Amplicon11 | 5′- TTCAGGCTCACATGTGGTTG -3′ | 5′- GCGTTCATCTTGGCTTGAGT -3′ | 375 |
| | (SEQUENCE:31) | (SEQUENCE:32) | |
| Amplicon12 | 5′- TACTCCAGCCTGGGCAACAA -3′ | 5′- CCCGACTCATGAGTCCTTAC -3′ | 702 |
| | (SEQUENCE:33) | (SEQUENCE:34) | |
| Amplicon13 | 5′- CATGTTGACCAGGCTAGTCT -3′ | 5′- GACTCCATCTCGTGACCAAA -3′ | 415 |
| | (SEQUENCE:35) | (SEQUENCE:36) | |
| Amplicon15 | 5′- GACCAGGAGTCAAGGTTATG -3′ | 5′- GCATTCACCTAATGCTGTCC -3′ | 393 |
| | (SEQUENCE:37) | (SEQUENCE:38) | |
| Amplicon16 | 5′- TGAATCCGGTACTCACCGTC -3′ | 5′- AGCCAGATCATTTCCGACGC -3′ | 792 |
| | (SEQUENCE:39) | (SEQUENCE:40) | |
| Amplicon17 | 5′- GAGTTTCTCTCCACCGTGAC -3′ | 5′- GACGACAATGGCAGTTCTCG -3′ | 748 |
| | (SEQUENCE:41) | (SEQUENCE:42) | |
| Amplicon18 | 5′- CCGTGGTCTCCTTGCACTTT -3′ | 5′- TGCCTGAGCTCAAACCATCC -3′ | 708 |
| | (SEQUENCE:43) | (SEQUENCE:44) | |

Table 2 discloses the position of mutations of the present invention in their respective Amplicons.

TABLE 2

| | | Position in Amplicon |
|---|---|---|
| | | (relative to 5′ end of |
| Mutation | Amplicon | PrimerA side of Amplicon) |
| 1 (SEQUENCE: 5) | Amplicon2 | 152 |
| 2 (SEQUENCE: 6) | Amplicon2 | 317 |
| 3 (SEQUENCE: 7) | Amplicon7 | 244 |
| 4 (SEQUENCE: 8) | Amplicon8 | 243 |
| 5 (SEQUENCE: 9) | Amplicon23 | 296 |
| 6 (SEQUENCE: 14) | Amplicon23 | 380 |
| 7 (SEQUENCE: 15) | Amplicon10 | 146 |
| 8 (SEQUENCE: 16) | Amplicon10 | 290 |
| 9 (SEQUENCE: 17) | Amplicon11 | 88 |
| 10 (SEQUENCE: 18) | Amplicon11 | 102 |
| 11 (SEQUENCE: 19) | Amplicon11 | 176 |
| 12 (SEQUENCE: 45) | Amplicon11 | 224 |
| 13 (SEQUENCE: 46) | Amplicon12 | 268 |
| 14 (SEQUENCE: 47) | Amplicon13 | 249 |
| 15 (SEQUENCE: 48) | Amplicon14 | 55 |
| 16 (SEQUENCE: 49) | Amplicon14 | 314 |
| 17 (SEQUENCE: 50) | Amplicon16 | 85 |
| 18 (SEQUENCE: 51) | Amplicon16 | 123 |
| 19 (SEQUENCE: 52) | Amplicon16 | 252 |
| 20 (SEQUENCE: 53) | Amplicon17 | 50 |
| 21 (SEQUENCE: 54) | Amplicon17 | 239 |
| 22 (SEQUENCE: 55) | Amplicon17 | 401 |
| 23 (SEQUENCE: 56) | Amplicon17 | 508 |
| 24 (SEQUENCE: 57) | Amplicon18 | 416 |
| 25 (SEQUENCE: 58) | Amplicon18 | 418 |
| 26 (SEQUENCE: 59) | Amplicon18 | 434-436 |
| 27 (SEQUENCE: 60) | Amplicon18 | 461 |
| 28 (SEQUENCE: 61) | Amplicon18 | 467 |

Nucleic Acid Sequence Analysis

Nucleic acid sequence analysis is approached by a combination of (a) physiochemical techniques, based on the hybridization or denaturation of a probe strand plus its complementary target, and (b) enzymatic reactions with endonucleases, ligases, and polymerases. Nucleic acid can be assayed at the DNA or RNA level. The former analyzes the genetic potential of individual humans and the latter the expressed information of particular cells.

In assays using nucleic acid hybridization, detecting the presence of a DNA duplex in a process of the present invention can be accomplished by a variety of means.

In one approach for detecting the presence of a DNA duplex, an oligonucleotide that is hybridized in the DNA duplex includes a label or indicating group that will render the duplex detectable. Typically such labels include radioactive atoms, chemically modified nucleotide bases, and the like.

The oligonucleotide can be labeled, i.e., operatively linked to an indicating means or group, and used to detect the presence of a specific nucleotide sequence in a target template.

Radioactive elements operatively linked to or present as part of an oligonucleotide probe (labeled oligonucleotide) provide a useful means to facilitate the detection of a DNA duplex. A typical radioactive element is one that produces beta ray emissions. Elements that emit beta rays, such as ³H, ¹²C, ³²P and ³⁵S represent a class of beta ray emission-producing radioactive element labels. A radioactive polynucleotide probe is typically prepared by enzymatic incorporation of radioactively labeled nucleotides into a nucleic acid using DNA kinase.

Alternatives to radioactively labeled oligonucleotides are oligonucleotides that are chemically modified to contain metal complexing agents, biotin-containing groups, fluorescent compounds, and the like.

One useful metal complexing agent is a lanthanide chelate formed by a lanthanide and an aromatic beta-diketone, the lanthanide being bound to the nucleic acid or oligonucleotide via a chelate-forming compound such as an EDTA-analogue so that a fluorescent lanthanide complex is formed. See U.S. Pat. Nos. 4,374,120, 4,569,790 and published Patent Application EP0139675 and WO87/02708.

Biotin or acridine ester-labeled oligonucleotides and their use to label polynucleotides have been described. See U.S. Pat. No. 4,707,404, published Patent Application EP0212951 and European Patent No. 0087636. Useful fluorescent marker compounds include fluorescein, rhodamine, Texas Red, NBD and the like.

A labeled oligonucleotide present in a DNA duplex renders the duplex itself labeled and therefore distinguishable over other nucleic acids present in a sample to be assayed. Detecting the presence of the label in the duplex and thereby the presence of the duplex, typically involves separating the DNA duplex from any labeled oligonucleotide probe that is not hybridized to a DNA duplex.

Techniques for the separation of single stranded oligonucleotide, such as non-hybridized labeled oligonucleotide probe, from DNA duplex are well known, and typically involve the separation of single stranded from double stranded nucleic acids on the basis of their chemical properties. More often separation techniques involve the use of a heterogeneous hybridization format in which the non-hybridized probe is separated, typically by washing, from the DNA duplex that is bound to an insoluble matrix. Exemplary is the Southern blot technique, in which the matrix is a nitrocellulose sheet and the label is ³²P Southern, J. Mol. Biol., 98:503 (1975).

The oligonucleotides can also be advantageously linked, typically at or near their 5′-terminus, to a solid matrix, i.e., aqueous insoluble solid support. Useful solid matrices are well known in the art and include cross-linked dextran such as that available under the tradename SEPHADEX from Pharmacia Fine Chemicals (Piscataway, N.J.); agarose, polystyrene or latex beads about 1 micron to about 5 millimeters in diameter, polyvinyl chloride, polystyrene, cross-linked polyacrylamide, nitrocellulose or nylon-based webs such as sheets, strips, paddles, plates, microtiter plate wells and the like.

It is also possible to add “linking” nucleotides to the 5′ or 3′ end of the member oligonucleotide, and use the linking oligonucleotide to operatively link the member to the solid support.

In nucleotide hybridizing assays, the hybridization reaction mixture is maintained in the contemplated method under hybridizing conditions for a time period sufficient for the oligonucleotides having complementarity to the predetermined sequence on the template to hybridize to complementary nucleic acid sequences present in the template to form a hybridization product, i.e., a complex containing oligonucleotide and target nucleic acid.

The phrase “hybridizing conditions” and its grammatical equivalents, when used with a maintenance time period, indicates subjecting the hybridization reaction admixture, in the context of the concentrations of reactants and accompanying reagents in the admixture, to time, temperature and pH conditions sufficient to allow one or more oligonucleotides to anneal with the target sequence, to form a nucleic acid duplex. Such time, temperature and pH conditions required to accomplish hybridization depend, as is well known in the art, on the length of the oligonucleotide to be hybridized, the degree of complementarity between the oligonucleotide and the target, the guanine and cytosine content of the oligonucleotide, the stringency of hybridization desired, and the presence of salts or additional reagents in the hybridization reaction admixture as may affect the kinetics of hybridization. Methods for optimizing hybridization conditions for a given hybridization reaction admixture are well known in the art.

Typical hybridizing conditions include the use of solutions buffered to pH values between 4 and 9, and are carried out at temperatures from 4° C. to 37° C., preferably about 12° C. to about 30° C., more preferably about 22° C., and for time periods from 0.5 seconds to 24 hours, preferably 2 minutes (min) to 1 hour.

Hybridization can be carried out in a homogeneous or heterogeneous format as is well known. The homogeneous hybridization reaction occurs entirely in solution, in which both the oligonucleotide and the nucleic acid sequences to be hybridized (target) are present in soluble forms in solution. A heterogeneous reaction involves the use of a matrix that is insoluble in the reaction medium to which either the oligonucleotide, polynucleotide probe or target nucleic acid is bound.

Where the nucleic acid containing a target sequence is in a double stranded (ds) form, it is preferred to first denature the dsDNA, as by heating or alkali treatment, prior to conducting the hybridization reaction. The denaturation of the dsDNA can be carried out prior to admixture with an oligonucleotide to be hybridized, or can be carried out after the admixture of the dsDNA with the oligonucleotide.

Predetermined complementarity between the oligonucleotide and the template is achieved in two alternative manners. A sequence in the template DNA may be known, such as where the primer to be formed can hybridize to known low density lipoprotein receptor sequences and can initiate primer extension into a region of DNA for sequencing purposes, as well as subsequent assaying purposes as described herein, or where previous sequencing has determined a region of nucleotide sequence and the primer is designed to extend from the recently sequenced region into a region of unknown sequence. This latter process has been referred to as “directed sequencing” because each round of sequencing is directed by a primer designed based on the previously determined sequence.

Effective amounts of the oligonucleotide present in the hybridization reaction admixture are generally well known and are typically expressed in terms of molar ratios between the oligonucleotide to be hybridized and the template. Preferred ratios are hybridization reaction mixtures containing equimolar amounts of the target sequence and the oligonucleotide. As is well known, deviations from equal molarity will produce hybridization reaction products, although at lower efficiency. Thus, although ratios where one component can be in as much as 100 fold molar excess relative to the other component, excesses of less than 50 fold, preferably less than 10 fold, and more preferably less than two fold are desirable in practicing the invention.

Detection of Membrane-Immobilized Target Sequences

In the DNA (Southern) blot technique, DNA is prepared by PCR amplification as previously discussed. The PCR products (DNA fragments) are separated according to size in an agarose gel and transferred (blotted) onto a nitrocellulose or nylon membrane. Conventional electrophoresis separates fragments ranging from 100 to 30,000 base pairs while pulsed field gel electrophoresis resolves fragments up to 20 million base pairs in length. The location on the membrane containing a particular PCR product is determined by hybridization with a specific, labeled nucleic acid probe.

In preferred embodiments, PCR products are directly immobilized onto a solid-matrix (nitrocellulose membrane) using a dot-blot (slot-blot) apparatus, and analyzed by probe-hybridization. See U.S. Pat. Nos. 4,582,789 and 4,617,261.

Immobilized DNA sequences may be analyzed by probing with allele-specific oligonucleotide (ASO) probes, which are synthetic DNA oligomers of approximately 15, 17, 20, 25 or up to about 30 nucleotides in length. These probes are long enough to represent unique sequences in the genome, but sufficiently short to be destabilized by an internal mismatch in their hybridization to a target molecule. Thus, any sequences differing at single nucleotides may be distinguished by the different denaturation behaviors of hybrids between the ASO probe and normal or mutant targets under carefully controlled hybridization conditions. Probes are suitable as long as they hybridize specifically to the region of the LDLR gene carrying the mutation of choice, and are capable of specifically distinguishing between a polynucleotide carrying the point mutation and a wild type polynucleotide.

Detection of Target Sequences in Solution

Several rapid techniques that do not require nucleic acid purification or immobilization have been developed. For example, probe/target hybrids may be selectively isolated on a solid matrix, such as hydroxylapatite, which preferentially binds double-stranded nucleic acids. Alternatively, probe nucleic acids may be immobilized on a solid support and used to capture target sequences from solution. Detection of the target sequences can be accomplished with the aid of a second, labeled probe that is either displaced from the support by the target sequence in a competition-type assay or joined to the support via the bridging action of the target sequence in a sandwich-type format.

In the oligonucleotide ligation assay (OLA), the enzyme DNA ligase is used to covalently join two synthetic oligonucleotide sequences selected so that they can base pair with a target sequence in exact head-to-tail juxtaposition. Ligation of the two oligomers is prevented by the presence of mismatched nucleotides at the junction region. This procedure allows for the distinction between known sequence variants in samples of cells without the need for DNA purification. The joining of the two oligonucleotides may be monitored by immobilizing one of the two oligonucleotides and observing whether the second, labeled oligonucleotide is also captured.

Scanning Techniques for Detection of Base Substitutions

Three techniques permit the analysis of probe/target duplexes several hundred base pairs in length for unknown single-nucleotide substitutions or other sequence differences. In the ribonuclease (RNase) A technique, the enzyme cleaves a labeled RNA probe at positions where it is mismatched to a target RNA or DNA sequence. The fragments may be separated according to size allowing for the determination of the approximate position of the mutation. See U.S. Pat. No. 4,946,773.

In the denaturing gradient gel technique, a probe-target DNA duplex is analyzed by electrophoresis in a denaturing gradient of increasing strength. Denaturation is accompanied by a decrease in migration rate. A duplex with a mismatched base pair denatures more rapidly than a perfectly matched duplex.

A third method relies on chemical cleavage of mismatched base pairs. A mismatch between T and C, G, or T, as well as mismatches between C and T, A, or C, can be detected in heteroduplexes. Reaction with osmium tetroxide (T and C mismatches) or hydroxylamine (C mismatches) followed by treatment with piperidine cleaves the probe at the appropriate mismatch.

Therapeutic Agents for Restoring and/or Enhancing LDLR Function

Where a mutation in the LDLR gene leads to defective LDLR function and this defective function is associated with increased susceptibility of a patient to pathogenic infection, whether through lower levels of LDLR protein, mutation in the protein affecting its function, or other mechanisms, it may be advantageous to treat the patient with wild type LDLR protein. Furthermore, if the mutation gives rise in infection-resistant carriers to a form of the protein that differs from the reference protein, and that has an advantage in terms of inhibiting HCV infection, it may be advantageous to administer a protein encoded by the mutated gene. In the case of LDLR, mutation appears to reduce binding of the virus to the cell surface and thereby inhibit infection. Therefore, it can be envisioned that any therapeutic strategy that inhibits this essential interaction between the virus and the cell would succeed in attenuating infection. One preferred strategy would involve the administration of wild-type LDLR, or fragments thereof, in excess in order to effectively compete for HCV particle binding with native LDLR on the cell surface. This soluble receptor strategy will be recognized by those skilled in the art. Furthermore, the present invention envisions polypeptides composed of or derived from the natural ligands of LDLR that competitively inhibit virus binding to and internalization by the receptor. Natural ligands of LDLR include LDL and VLDL and their protein components, APOB and APOE respectively. The APOB and APOE proteins as defined by SEQUENCE:12 and SEQUENCE:13 and polypeptide derivatives thereof are envisioned as possible inhibitors of virus binding, entry and infection by the present invention. The discussion below pertains to administration of any of the foregoing proteins or polypeptides.

The polypeptides of the present invention, including those encoded by mutant or wild-type LDLR, may be a naturally purified product, or a product of chemical synthetic procedures, or produced by recombinant techniques from a prokaryotic or eukaryotic host (for example, by bacterial, yeast, higher plant, insect and mammalian cells in culture) of a polynucleotide sequence of the present invention. Depending upon the host employed in a recombinant production procedure, the polypeptides of the present invention may be glycosylated with mammalian or other eukaryotic carbohydrates or may be non-glycosylated. The polypeptides of the current invention may also be myristylated or have other post-translational modifications. Polypeptides of the invention may also include an initial methionine amino acid residue (at position minus 1) which may be formulated to contain a Kozak consensus sequence.

The polypeptides of the present invention also include the protein sequences defined in SEQUENCE:10, SEQUENCE:11, SEQUENCE:12, SEQUENCE:13, SEQUENCE:20, and derivatives thereof.

In addition to naturally occurring allelic forms of the polypeptide(s), the present invention also embraces analogs and fragments thereof, which function similarly to the naturally occurring allelic forms. Thus, for example, one or more of the amino acid residues of the polypeptide may be replaced by conserved amino acid residues, as long as the function of the mutant or wild-type LDLR protein is maintained. For example, in the preferred soluble receptor strategy described above, the fragment of LDLR that normally binds with high efficiency to the HCV nucleocapsid particle could be manufactured and administered for the purpose of treating HCV infection. In another preferred embodiment, fragments of APOB or APOE that competitively inhibit virus infection could be manufactured and administered for the purpose of treating HCV infection.

The polypeptides may also be employed in accordance with the present invention by expression of such polypeptides in vivo, which is often referred to as gene therapy. Thus, for example, cells may be transduced with a polynucleotide (DNA or RNA) encoding the polypeptides ex vivo with those transduced cells then being provided to a patient to be treated with the polypeptide. Such methods are well known in the art. For example, cells may be transduced by procedures known in the art by use of a retroviral particle containing RNA encoding the polypeptide of the present invention. Additional examples involve the use of lentivirus and adenovirus-derived vectors and genetically engineered stem cells.

Similarly, transduction of cells may be accomplished in vivo for expression of the polypeptide in vivo, for example, by procedures known in the art. As known in the art, a producer cell for producing a retroviral particle containing RNA encoding the polypeptides of the present invention may be administered to a patient for transduction in vivo and expression of the polypeptides in vivo.

These and other methods for administering the polypeptides of the present invention by such methods should be apparent to those skilled in the art from the teachings of the present invention. For example, the expression vehicle for transducing cells may be other than a retrovirus, for example, an adenovirus which may be used to transduce cells in vivo after combination with a suitable delivery vehicle. Transduction of gene therapy vectors may also be accomplished by formulation into liposomes or a similar carrier. Conjugation to copolymers such as N-(2-hydroxypropyl) methacrylamide (HPMA) or polyethylene glycol (PEG) for the purposes of vector delivery or to improve the pharmacokinetics or pharmacodynamics of gene therapy reagents is also envisioned by the present invention. As one skilled in the art will recognize, many such derivatizations are possible.

Furthermore, as is known in the art, both the polypeptides and gene therapy vectors of the present invention can be conjugated to polybasic polypeptide transduction domains to facilitate delivery to the target organ or target subcellular location. Such polybasic polypeptide transduction domains include but are not limited to the HIV transactivator of transcription (TAT) protein transduction domain, VP22, polyarginine, polylysine, penetratin, and others.

In the case where the polypeptides are prepared as a liquid formulation and administered by injection, preferably the solution is an isotonic salt solution containing 140 millimolar sodium chloride and 10 millimolar calcium at pH 7.4. The injection may be administered, for example, in a therapeutically effective amount, preferably in a dose of about 1 μg/kg body weight to about 5 mg/kg body weight daily, taking into account the routes of administration, health of the patient, etc.

The polypeptide(s) of the present invention may be employed in combination with a suitable pharmaceutical carrier. Such compositions comprise a therapeutically effective amount, of the protein, and a pharmaceutically acceptable carrier or excipient. Such a carrier includes but is not limited to saline, buffered saline, dextrose, water, glycerol, ethanol, and combinations thereof. The formulation should suit the mode of administration.

The polypeptide(s) of the present invention can also be modified by chemically linking the polypeptide to one or more moieties or conjugates to enhance the activity, cellular distribution, or cellular uptake of the polypeptide(s). Such moieties or conjugates include lipids such as cholesterol, cholic acid, thioether, aliphatic chains, phospholipids and their derivatives, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

The polypeptide(s) of the present invention may also be modified to target specific cell types for a particular disease indication, including but not limited to liver cells in the case of hepatitis C infection. As can be appreciated by those skilled in the art, suitable methods have been described that achieve the described targeting goals and include, without limitation, liposomal targeting, receptor-mediated endocytosis, and antibody-antigen binding. In one embodiment, the asiaglycoprotein receptor may be used to target liver cells by the addition of a galactose moiety to the polypeptide(s). In another embodiment, mannose moieties may be conjugated to the polypeptide(s) in order to target the mannose receptor found on macrophages and liver cells. The polypeptide(s) of the present invention may also be modified for cytosolic delivery by methods known to those skilled in the art, including, but not limited to, endosome escape mechanisms or protein transduction domain (PTD) systems. Known endosome escape systems include the use of pH-responsive polymeric carriers such as poly(propylacrylic acid). Known PTD systems range from natural peptides such as HIV-1 TAT or HSV-1 VP22, to synthetic peptide carriers. As one skilled in the art will recognize, multiple delivery and targeting methods may be combined. For example, the polypeptide(s) of the present invention may be targeted to liver cells by encapsulation within liposomes, such liposomes being conjugated to galactose for targeting to the asialoglycoprotein receptor.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptide(s) of the present invention may be employed in conjunction with other therapeutic compounds.

When the LDLR reference protein or variant proteins of the present invention are used as a pharmaceutical, they can be given to mammals, in a suitable vehicle. When the polypeptides of the present invention are used as a pharmaceutical as described above, they are given, for example, in therapeutically effective doses of about 10 μg/kg body weight to about 100 mg/kg body weight daily, taking into account the routes of administration, health of the patient, etc. The amount given is preferably adequate to achieve prevention or inhibition of infection by a virus, preferably an RNA virus, preferably a positive stand RNA virus, preferably a flavivirus, preferably HCV, thus replicating the natural resistance found in humans carrying a mutant LDLR allele as disclosed herein.

Inhibitor-based drug therapies that mimic the beneficial effects (i.e. resistance to infection) of at least one mutation at position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of NT_{—}011295.10 are also envisioned, as discussed in detail below. These inhibitor-based therapies can take the form of chemical entities, peptides or proteins, antisense oligonucleotides, small interference RNAs, and antibodies.

The proteins, their fragments or other derivatives, or analogs thereof, or cells expressing them can be used as an immunogen to produce antibodies thereto. These antibodies can be, for example, polyclonal, monoclonal, chimeric, single chain, Fab fragments, or the product of a Fab expression library. Various procedures known in the art may be used for the production of polyclonal antibodies.

Antibodies generated against the polypeptide encoded by mutant or reference LDLR of the present invention can be obtained by direct injection of the polypeptide into an animal or by administering the polypeptide to an animal, preferably a nonhuman. The antibody so obtained will then bind the polypeptide itself. In this manner, even a sequence encoding only a fragment of the polypeptide can be used to generate antibodies binding the whole native polypeptide. Moreover, a panel of such antibodies, specific to a large number of polypeptides, can be used to identify and differentiate such tissue.

For preparation of monoclonal antibodies, any technique which provides antibodies produced by continuous cell line cultures can be used. Examples include the hybridoma technique (Kohler and Milstein, 1975, Nature, 256:495-597), the trioma technique, the human B-cell hybridoma technique (Kozbor, et al., 1983, Immunology Today 4:72), and the EBV-hybridoma technique to produce human monoclonal antibodies (Coe, et al., 1985, Monoclonal Antibodies and Cancer Therapy, Alan R. Liss, Inc. pp. 77-96).

Techniques described for the production of single chain antibodies (U.S. Pat. No. 4,946,778) can be adapted to produce single chain antibodies to immunogenic polypeptide products of this invention.

The antibodies can be used in methods relating to the localization and activity of the protein sequences of the invention, e.g., for imaging these proteins, measuring levels thereof in appropriate physiological samples, and the like. Antibodies can also be used therapeutically to inhibit viral infection by inhibiting the interaction between the virus and LDLR. As one skilled in the art will recognize, therapeutic antibodies can be humanized by a number of well known methods in order to reduce their inflammatory potential.

The present invention provides detectably labeled oligonucleotides for imaging LDLR polynucleotides within a cell. Such oligonucleotides are useful for determining if gene amplification has occurred, and for assaying the expression levels in a cell or tissue using, for example, in situ hybridization as is known in the art.

Therapeutic Agents for Inhibition of LDLR Function

The present invention also relates to antisense oligonucleotides designed to interfere with the normal function of LDLR polynucleotides. Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773.

The antisense compounds of the invention can include modified bases as disclosed in U.S. Pat. No. 5,958,773 and patents disclosed therein. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

Preferred antisense oligonucleotides can be selected by routine experimentation using, for example, assays described in the Examples. Although the inventors are not bound by a particular mechanism of action, it is believed that the antisense oligonucleotides achieve an inhibitory effect by binding to a complementary region of the target polynucleotide within the cell using Watson-Crick base pairing. Where the target polynucleotide is RNA, experimental evidence indicates that the RNA component of the hybrid is cleaved by RNase H (Giles et al., Nuc. Acids Res. 23:954-61, 1995; U.S. Pat. No. 6,001,653). Generally, a hybrid containing 10 base pairs is of sufficient length to serve as a substrate for RNase H. However, to achieve specificity of binding, it is preferable to use an antisense molecule of at least 17 nucleotides, as a sequence of this length is likely to be unique among human genes.

As disclosed in U.S. Pat. No. 5,998,383, incorporated herein by reference, the oligonucleotide is selected such that the sequence exhibits suitable energy related characteristics important for oligonucleotide duplex formation with their complementary templates, and shows a low potential for self-dimerization or self-complementation (Anazodo et al., Biochem. Biophys. Res. Commun. 229:305-09, 1996). The computer program OLIGO (Primer Analysis Software, Version 3.4), is used to determined antisense sequence melting temperature, free energy properties, and to estimate potential self-dimer formation and self-complimentarity properties. The program allows the determination of a qualitative estimation of these two parameters (potential self-dimer formation and self-complimentary) and provides an indication of “no potential” or “some potential” or “essentially complete potential.” Segments of LDLR polynucleotides are generally selected that have estimates of no potential in these parameters. However, segments can be used that have “some potential” in one of the categories. A balance of the parameters is used in the selection.

In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. According to the invention, this experimentation can be performed routinely by transfecting cells with an antisense oligonucleotide using methods described in the Examples. mRNA levels in the cell can be measured routinely in treated and control cells by reverse transcription of the mRNA and assaying the cDNA levels. The biological effect can be determined routinely by measuring cell growth or viability as is known in the art.

Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. 23:45-50, 1998.) According to the present invention, cultures of cells are transfected with two different antisense oligonucleotides designed to target LDLR. The levels of mRNA corresponding to LDLR are measured in treated and control cells.

Additional inhibitors include ribozymes, proteins or polypeptides, antibodies or fragments thereof as well as small molecules. Each of these LDLR inhibitors share the common feature in that they reduce the expression and/or biological activity of LDLR or specifically inhibit the interaction of virus with LDLR thereby preventing, attenuating or curing infection. In addition to the exemplary LDLR inhibitors disclosed herein, alternative inhibitors may be obtained through routine experimentation utilizing methodology either specifically disclosed herein or as otherwise readily available to and within the expertise of the skilled artisan.

Ribozymes

LDLR inhibitors may be ribozymes. A ribozyme is an RNA molecule that specifically cleaves RNA substrates, such as mRNA, resulting in specific inhibition or interference with cellular gene expression. As used herein, the term ribozymes includes RNA molecules that contain antisense sequences for specific recognition, and an RNA-cleaving enzymatic activity. The catalytic strand cleaves a specific site in a target RNA at greater than stoichiometric concentration.

A wide variety of ribozymes may be utilized within the context of the present invention, including for example, the hammerhead ribozyme (for example, as described by Forster and Symons, Cell 48:211-20, 1987; Haseloff and Gerlach, Nature 328:596-600, 1988; Walbot and Bruening, Nature 334:196, 1988; Haseloff and Gerlach, Nature 334:585, 1988); the hairpin ribozyme (for example, as described by Haseloff et al., U.S. Pat. No. 5,254,678, issued Oct. 19, 1993 and Hempel et al., European Patent Publication No. 0 360 257, published Mar. 26, 1990); and Tetrahymena ribosomal RNA-based ribozymes (see Cech et al., U.S. Pat. No. 4,987,071). Ribozymes of the present invention typically consist of RNA, but may also be composed of DNA, nucleic acid analogs (e.g., phosphorothioates), or chimerics thereof (e.g., DNA/RNA/RNA).

Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). According to certain embodiments of the invention, any such LDLR mRNA-specific ribozyme, or a nucleic acid encoding such a ribozyme, may be delivered to a host cell to effect inhibition of LDLR gene expression. Ribozymes and the like may therefore be delivered to the host cells by DNA encoding the ribozyme linked to a eukaryotic promoter, such as a eukaryotic viral promoter, such that upon introduction into the nucleus, the ribozyme will be directly transcribed.

RNAi

The invention also provides for the introduction of RNA with partial or fully double-stranded character into the cell or into the extracellular environment. Inhibition is specific to the LDLR expression in that a nucleotide sequence from a portion of the target LDLR gene is chosen to produce inhibitory RNA. This process is (1) effective in producing inhibition of gene expression, and (2) specific to the targeted LDLR gene. The procedure may provide partial or complete loss of function for the target LDLR gene. A reduction or loss of gene expression in at least 99% of targeted cells has been shown using comparable techniques with other target genes. Lower doses of injected material and longer times after administration of dsRNA may result in inhibition in a smaller fraction of cells. Quantitation of gene expression in a cell may show similar amounts of inhibition at the level of accumulation of target mRNA or translation of target protein. Methods of preparing and using RNAi are generally disclosed in U.S. Pat. No. 6,506,559, incorporated herein by reference.

The RNA may comprise one or more strands of polymerized ribonucleotide; it may include modifications to either the phosphate-sugar backbone or the nucleoside. The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. RNA containing a nucleotide sequence identical to a portion of the LDLR target gene is preferred for inhibition. RNA sequences with insertions, deletions, and single point mutations relative to the target sequence have also been found to be effective for inhibition. Thus, sequence identity may be optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region may be used to transcribe the RNA strand (or strands).

For RNAi, the RNA may be directly introduced into the cell (i.e., intracellularly), or introduced extracellularly into a cavity, interstitial space, into the circulation of an organism, introduced orally, or may be introduced by bathing an organism in a solution containing RNA. Methods for oral introduction include direct mixing of RNA with food of the organism, as well as engineered approaches in which a species that is used as food is engineered to express an RNA, then fed to the organism to be affected. Physical methods of introducing nucleic acids include injection directly into the cell or extracellular injection into the organism of an RNA solution.

The advantages of the method include the ease of introducing double-stranded RNA into cells, the low concentration of RNA which can be used, the stability of double-stranded RNA, and the effectiveness of the inhibition.

As one skilled in the art will recognize, all of the above methods, RNAi, ribozyme, and antisense, can be designed to bind to and inhibit the expression of one specific allele of the LDLR gene by virtue of discriminating one or more of the mutations at position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of NT_{—}011295.10. Such an approach can be used to modulate the relative expression of one allele over the other, favoring expression of alleles of LDLR that confer resistance to HCV infection.

Inhibition of gene expression refers to the absence (or observable decrease) in the level of protein and/or mRNA product from a LDLR target gene. Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression is conveniently assayed by use of a reporter or drug resistance gene whose protein product is easily assayed. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin.

Depending on the assay, quantitation of the amount of gene expression allows one to determine a degree of inhibition which is greater than 10%, 33%, 50%, 90%, 95% or 99% as compared to a cell not treated according to the present invention. Lower doses of injected material and longer times after administration of dsRNA may result in inhibition in a smaller fraction of cells (e.g., at least 10%, 20%, 50%, 75%, 90%, or 95% of targeted cells). Quantitation of LDLR gene expression in a cell may show similar amounts of inhibition at the level of accumulation of LDLR target mRNA or translation of LDLR target protein. As an example, the efficiency of inhibition may be determined by assessing the amount of gene product in the cell: mRNA may be detected with a hybridization probe having a nucleotide sequence outside the region used for the inhibitory double-stranded RNA, or translated polypeptide may be detected with an antibody raised against the polypeptide sequence of that region.

The RNA may comprise one or more strands of polymerized ribonucleotide. It may include modifications to either the phosphate-sugar backbone or the nucleoside. For example, the phosphodiester linkages of natural RNA may be modified to include at least one of a nitrogen or sulfur heteroatom. Modifications in RNA structure may be tailored to allow specific genetic inhibition while avoiding a general panic response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition; lower doses may also be useful for specific applications. Inhibition is sequence-specific in that nucleotide sequences corresponding to the duplex region of the RNA are targeted for genetic inhibition.

RNA containing nucleotide sequences identical to a portion of the LDLR target gene are preferred for inhibition. RNA sequences with insertions, deletions, and single point mutations relative to the target sequence may be effective for inhibition. Thus, sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the LDLR target gene is preferred. Alternatively, the duplex region of the RNA may be defined functionally as a nucleotide sequence that is capable of hybridizing with a portion of the LDLR target gene transcript (e.g., 400 mM NaCl, 40 mM PIPES pH 6.4, 1 mM EDTA, 50° C. or 70° C. hybridization for 12-16 hours; followed by washing). The length of the identical nucleotide sequences may be at least 25, 50, 100, 200, 300 or 400 bases.

100% sequence identity between the RNA and the LDLR target gene is not required to practice the present invention. Thus the methods have the advantage of being able to tolerate sequence variations that might be expected due to genetic mutation, strain polymorphism, or evolutionary divergence.

LDLR RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (see WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

RNA may be directly introduced into the cell (i.e., intracellularly); or introduced extracellularly into a cavity, interstitial space, into the circulation of an organism, introduced orally, by subcutaneous, intramuscular, intravenous, or intraperitoneal injection, transdermally, or may be introduced by bathing an organism in a solution containing the RNA. Methods for oral introduction include direct mixing of the RNA with food of the organism, as well as engineered approaches in which a species that is used as food is engineered to express the RNA, then fed to the organism to be affected. For example, the RNA may be sprayed onto a plant or a plant may be genetically engineered to express the RNA in an amount sufficient to kill some or all of a pathogen known to infect the plant. Physical methods of introducing nucleic acids, for example, injection directly into the cell or extracellular injection into the organism, may also be used. Vascular or extravascular circulation, the blood or lymph system, and the cerebrospinal fluid are sites where the RNA may be introduced. A transgenic organism that expresses RNA from a recombinant construct may be produced by introducing the construct into a zygote, an embryonic stem cell, or another multipotent cell derived from the appropriate organism.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

The present invention may be used alone or as a component of a kit having at least one of the reagents necessary to carry out the in vitro or in vivo introduction of RNA to test samples or subjects. Preferred components are the dsRNA and a vehicle that promotes introduction of the dsRNA. Such a kit may also include instructions to allow a user of the kit to practice the invention.

Suitable injection mixes are constructed so animals receive an average of 0.5×10⁶ to 1.0×10⁶ molecules of RNA. For comparisons of sense, antisense, and dsRNA activities, injections are compared with equal masses of RNA (i.e., dsRNA at half the molar concentration of the single strands). Numbers of molecules injected per adult are given as rough approximations based on concentration of RNA in the injected material (estimated from ethidium bromide staining) and injection volume (estimated from visible displacement at the site of injection). A variability of several-fold in injection volume between individual animals is possible.

Proteins and Polypeptides

In addition to the antisense molecules and ribozymes disclosed herein, LDLR inhibitors of the present invention also include proteins or polypeptides that are effective in either reducing LDLR gene expression or in decreasing one or more of LDLR's biological activities, including but not limited to its ability to bind LDL- or VLDL-C, apolipoprotein B, apolipoprotein E, HCV nucleocapsid particles, other virus particles, HCV E2 protein, other virus envelop proteins; to encapsulate in clathirin coated vesicles; to undergo conformational changes necessary for endosomal release of ligand or HCV particles; or to recycle to the plasma membrane A variety of methods are readily available in the art by which the skilled artisan may, through routine experimentation, rapidly identify such LDLR inhibitors. The present invention is not limited by the following exemplary methodologies.

Literature is available to the skilled artisan that describes methods for detecting and analyzing protein-protein interactions. Reviewed in Phizicky et al., Microbiological Reviews 59:94-123, 1995, incorporated herein by reference. Such methods include, but are not limited to physical methods such as, e.g., protein affinity chromatography, affinity blotting, immunoprecipitation and cross-linking as well as library-based methods such as, e.g., protein probing, phage display and two-hybrid screening. Other methods that may be employed to identify protein-protein interactions include genetic methods such as use of extragenic or second-site suppressors, synthetic lethal effects and unlinked noncomplementation. Exemplary methods are described in further detail below.

Inventive LDLR inhibitors may be identified through biological screening assays that rely on the direct interaction between the LDLR protein and/or the polypeptides of SEQUENCE:10, SEQUENCE:11, or SEQUENCE:20 and a panel or library of potential inhibitor proteins. Biological screening methodologies, including the various “n-hybrid technologies,” are described in, for example, Vidal et al., Nucl. Acids Res. 27(4):919-29, 1999; Frederickson, R. M., Curr. Opin. Biotechnol. 9(1):90-96, 1998; Brachmann et al., Curr. Opin. Biotechnol. 8(5):561-68, 1997; and White, M. A., Proc. Natl. Acad. Sci. U.S.A. 93:10001-03, 1996, each of which is incorporated herein by reference.

The two-hybrid screening methodology may be employed to search new or existing target cDNA libraries for LDLR binding proteins that have inhibitory properties. The two-hybrid system is a genetic method that detects protein-protein interactions by virtue of increases in transcription of reporter genes. The system relies on the fact that site-specific transcriptional activators have a DNA-binding domain and a transcriptional activation domain. The DNA-binding domain targets the activation domain to the specific genes to be expressed. Because of the modular nature of transcriptional activators, the DNA-binding domain may be severed covalently from the transcriptional activation domain without loss of activity of either domain. Furthermore, these two domains may be brought into juxtaposition by protein-protein contacts between two proteins unrelated to the transcriptional machinery. Thus, two hybrids are constructed to create a functional system. The first hybrid, i.e., the bait, consists of a transcriptional activator DNA-binding domain fused to a protein of interest. The second hybrid, the target, is created by the fusion of a transcriptional activation domain with a library of proteins or polypeptides. Interaction between the bait protein and a member of the target library results in the juxtaposition of the DNA-binding domain and the transcriptional activation domain and the consequent up-regulation of reporter gene expression.

A variety of two-hybrid based systems are available to the skilled artisan that most commonly employ either the yeast Gal4 or E. coli LexA DNA-binding domain (BD) and the yeast Gal4 or herpes simplex virus VP16 transcriptional activation domain. Chien et al., Proc. Natl. Acad. Sci. U.S.A. 88:9578-82, 1991; Dalton et al., Cell 68:597-612, 1992; Durfee et al., Genes Dev. 7:555-69, 1993; Vojtek et al., Cell 74:205-14, 1993; and Zervos et al., Cell 72:223-32, 1993. Commonly used reporter genes include the E. coli lacZ gene as well as selectable yeast genes such as HIS3 and LEU2. Fields et al., Nature (London) 340:245-46, 1989; Durfee, T. K., supra; and Zervos, A. S., supra. A wide variety of activation domain libraries is readily available in the art such that the screening for interacting proteins may be performed through routine experimentation.

Suitable bait proteins for the identification of LDLR interacting proteins may be designed based on proteins encoded by the LDLR DNA sequence presented herein as SEQUENCE:1, and in a preferred embodiment, the polypeptides of SEQUENCE:10 or SEQUENCE:11. Such bait proteins include either the full-length LDLR protein or fragments thereof.

Plasmid vectors, such as, e.g., pBTM116 and pAS2-1, for preparing LDLR bait constructs and target libraries are readily available to the artisan and may be obtained from such commercial sources as, e.g., Clontech (Palo Alto, Calif.), Invitrogen (Carlsbad, Calif.) and Stratagene (La Jolla, Calif.). These plasmid vectors permit the in-frame fusion of cDNAs with the DNA-binding domains as LexA or Gal4BD, respectively.

LDLR inhibitors of the present invention may alternatively be identified through one of the physical or biochemical methods available in the art for detecting protein-protein interactions.

Through the protein affinity chromatography methodology, lead compounds to be tested as potential LDLR inhibitors may be identified by virtue of their specific retention to LDLR or polypeptide derivatives of LDLR when either covalently or non-covalently coupled to a solid matrix such as, e.g., Sepharose beads. The preparation of protein affinity columns is described in, for example, Beeckmans et al., Eur. J. Biochem. 117:527-35, 1981, and Formosa et al., Methods Enzymol. 208:24-45, 1991. Cell lysates containing the full complement of cellular proteins may be passed through the LDLR affinity column. Proteins having a high affinity for LDLR will be specifically retained under low-salt conditions while the majority of cellular proteins will pass through the column. Such high affinity proteins may be eluted from the immobilized LDLR under conditions of high-salt, with chaotropic solvents or with sodium dodecyl sulfate (SDS). In some embodiments, it may be preferred to radiolabel the cells prior to preparing the lysate as an aid in identifying the LDLR specific binding proteins. Methods for radiolabeling mammalian cells are well known in the art and are provided, e.g., in Sopta et al., J. Biol. Chem. 260:10353-60, 1985.

Suitable LDLR proteins for affinity chromatography may be fused to a protein or polypeptide to permit rapid purification on an appropriate affinity resin. For example, the LDLR cDNA may be fused to the coding region for glutathione S-transferase (GST) which facilitates the adsorption of fusion proteins to glutathione-agarose columns. Smith et al., Gene 67:31-40, 1988. Alternatively, fusion proteins may include protein A, which can be purified on columns bearing immunoglobulin G; oligohistidine-containing peptides, which can be purified on columns bearing Ni²⁺; the maltose-binding protein, which can be purified on resins containing amylose; and dihydrofolate reductase, which can be purified on methotrexate columns. One exemplary tag suitable for the preparation of LDLR fusion proteins that is presented herein is the epitope for the influenza virus hemagglutinin (HA) against which monoclonal antibodies are readily available and from which antibodies an affinity column may be prepared.

Proteins that are specifically retained on a LDLR affinity column may be identified after subjecting to SDS polyacrylamide gel electrophoresis (SDS-PAGE). Thus, where cells are radiolabeled prior to the preparation of cell lysates and passage through the LDLR affinity column, proteins having high affinity for LDLR may be detected by autoradiography. The identity of LDLR specific binding proteins may be determined by protein sequencing techniques that are readily available to the skilled artisan, such as Mathews, C. K. et al., Biochemistry, The Benjamin/Cummings Publishing Company, Inc., 1990, pp. 166-70. As one skilled in the art will recognize, numerous techniques of protein identification exist including various forms of mass spectroscopic analysis.

Small Molecules

The present invention also provides small molecule LDLR inhibitors that may be readily identified through routine application of high-throughput screening (HTS) methodologies. Reviewed by Persidis, A., Nature Biotechnology 16:488-89, 1998. HTS methods generally refer to those technologies that permit the rapid assaying of lead compounds, such as small molecules, for therapeutic potential. HTS methodology employs robotic handling of test materials, detection of positive signals and interpretation of data. Such methodologies include, e.g., robotic screening technology using soluble molecules as well as cell-based systems such as the two-hybrid system described in detail above.

A variety of cell line-based HTS methods are available that benefit from their ease of manipulation and clinical relevance of interactions that occur within a cellular context as opposed to in solution. Lead compounds may be identified via incorporation of radioactivity or through optical assays that rely on absorbance, fluorescence or luminescence as read-outs. See, e.g., Gonzalez et al., Curr. Opin. Biotechnol. 9(6):624-31, 1998, incorporated herein by reference.

HTS methodology may be employed, e.g., to screen for lead compounds that block one of LDLR's biological activities or that simply bind with high affinity to LDLR or specific regions of the LDLR protein, as in the preferred embodiment where compounds are identified that bind to the polypeptides of SEQUENCE:11, SEQUENCE:12, and SEQUENCE:20. By this method, LDLR protein may be immunoprecipitated or otherwise purified from cells expressing the protein and applied to wells on an assay plate suitable for robotic screening. LDLR or fragments thereof may also be expressed and purified using recombinant DNA technologies. Individual test compounds may then be contacted with the immunoprecipitated or purified protein and the effect of each test compound on LDLR measured.

Methods for Assessing the Efficacy of LDLR Inhibitors

Lead molecules or compounds, whether antisense molecules or ribozymes, proteins and/or peptides, antibodies and/or antibody fragments, small molecules, or derivatives of native LDLR ligand proteins (e.g. APOB or APOE) that are identified either by one of the methods described herein or via techniques that are otherwise available in the art, may be further characterized in a variety of in vitro, ex vivo and in vivo animal model assay systems for their ability to inhibit LDLR gene expression or biological activity. As discussed in further detail in the Examples provided below, LDLR inhibitors of the present invention are effective in reducing LDLR expression levels. Thus, the present invention further discloses methods that permit the skilled artisan to assess the effect of candidate inhibitors.

In other preferred embodiments, LDLR inhibitors are assessed for their ability to inhibit binding of HCV, HCV E2 protein, or the natural LDLR ligands (low-density lipoprotein, very low density lipoprotein, apolipoprotein B, and apolipoprotein E) to LDLR. As one skilled in the art will recognize, a variety of cell based and cell free methods can be used to assess the ability of inhibitors to bind to and inhibit the biological functions of LDLR. As one skilled in the art will recognize, inhibitors that preserve cholesterol metabolism while inhibiting viral binding and infection are preferred.

Candidate LDLR inhibitors may be tested by administration to cells that either express endogenous LDLR or that are made to express LDLR by transfection of a mammalian cell with a recombinant LDLR plasmid construct.

Effective LDLR inhibitory molecules will be effective in reducing the ability of LDLR to bind to the HCV nucleocapsid particle—or other virus particle—while not inhibiting any of the normal functions of the receptor (e.g. in cholesterol metabolism). Methods of measuring LDLR biological activity and HCV binding ability are known in the art, for example, as described in Agnello, V, et al., Proc Natl Acad Sci USA. 96(22):12766-71, 1999; and Wunschmann, S, et al. J Virol. 74(21): 10055-62, 2000), incorporated herein by reference.

The effectiveness of a given candidate antisense molecule or inhibitor may be assessed by comparison with a control “antisense” molecule or inhibitor known to have no substantial effect on LDLR expression or function when administered to a mammalian cell.

LDLR inhibitors effective in reducing LDLR gene expression or function by one or more of the methods discussed above may be further characterized in vitro for efficacy in one of the readily available established cell culture or primary cell culture model systems as described herein, in reference to use of Vero cells challenged by infection with a flavivirus, such as dengue virus.

Pharmaceutical Compositions

The antisense molecules and inhibitors of the present invention can be synthesized by any method known in the art, and final purity of the compositions is determined as is known in the art.

Therefore, pharmaceutical compositions and methods are provided for interfering with virus infection, preferably RNA virus infection, preferably positive strand RNA virus infection, preferably flavivirus, most preferably HCV infection, comprising contacting tissues or cells with one or more of the antisense or inhibitor compositions identified using the methods of the invention.

The invention provides pharmaceutical compositions of antisense oligonucleotides and ribozymes complementary to the LDLR mRNA gene sequence as active ingredients for therapeutic application. These compositions can also be used in the method of the present invention. When required, the compounds are nuclease resistant. In general the pharmaceutical composition for inhibiting virus infection in a mammal includes an effective amount of at least one antisense oligonucleotide as described above needed for the practice of the invention, or a fragment thereof shown to have the same effect, and a pharmaceutically physiologically acceptable carrier or diluent.

The compositions (LDLR inhibitors) can be administered orally, subcutaneously, transdermally, or parenterally including intravenous, intraarterial, intramuscular, intraperitoneally, and intranasal administration, as well as intrathecal and infusion techniques as required. The pharmaceutically acceptable carriers, diluents, adjuvants and vehicles as well as implant carriers generally refer to inert, non-toxic solid or liquid fillers, diluents or encapsulating material not reacting with the active ingredients of the invention. Cationic lipids may also be included in the composition to facilitate inhibitor uptake. Implants of the compounds are also useful. In general, the pharmaceutical compositions are sterile.

By bioactive (expressible) is meant that the antisense molecule or inhibitor is biologically active in the cell when delivered directly to the cell and/or, in the case of antisense molecules, is expressed by an appropriate promotor and active when delivered to the cell in a vector as described below. Nuclease resistance is provided by any method known in the art that does not substantially interfere with biological activity as described herein.

“Contacting the cell” refers to methods of exposing or delivering to a cell antisense oligonucleotides or inhibitors whether directly or by viral or non-viral vectors and where the antisense oligonucleotide or inhibitor is bioactive upon delivery.

The nucleotide sequences of the present invention can be delivered either directly or with viral or non-viral vectors. When delivered directly the sequences are generally rendered nuclease resistant. Alternatively, the sequences can be incorporated into expression cassettes or constructs such that the sequence is expressed in the cell. Generally, the construct contains the proper regulatory sequence or promotor to allow the sequence to be expressed in the targeted cell.

Once the oligonucleotide sequences are ready for delivery they can be introduced into cells as is known in the art. Transfection, electroporation, fusion, liposomes, colloidal polymeric particles, protein transduction technologies, and viral vectors as well as other means known in the art may be used to deliver the oligonucleotide sequences to the cell. The method selected will depend at least on the cells to be treated and the location of the cells and will be known to those skilled in the art. Localization can be achieved by liposomes, having specific markers on the surface for directing the liposome, by having injection directly into the tissue containing the target cells (e.g. by injection into the portal vein), by having depot associated in spatial proximity with the target cells, specific receptor mediated uptake, viral vectors, or the like.

The present invention provides vectors comprising an expression control sequence operatively linked to the oligonucleotide sequences of the invention. The present invention further provides host cells, selected from suitable eukaryotic and prokaryotic cells, which are transformed with these vectors as necessary.

Vectors are known or can be constructed by those skilled in the art and should contain all expression elements necessary to achieve the desired transcription of the sequences. Other beneficial characteristics can also be contained within the vectors such as mechanisms for recovery of the oligonucleotides in a different form. Phagemids are a specific example of such beneficial vectors because they can be used either as plasmids or as bacteriophage vectors. Examples of other vectors include viruses such as bacteriophages, baculoviruses and retroviruses, DNA viruses, liposomes and other recombination vectors. The vectors can also contain elements for use in either prokaryotic or eukaryotic host systems. Vectors can be used to transform or genetically engineer stem cells for implant into an organism. One of ordinary skill in the art will know which host systems are compatible with a particular vector.

The vectors can be introduced into cells or tissues by any one of a variety of known methods within the art. Such methods can be found generally described in Sambrook et al., Molecular Cloning. A Laboratory Manual, Cold Springs Harbor Laboratory, New York, 1989, 1992; in Ausubel et al., Current Protocols in Molecular Biology, John Wiley and Sons, Baltimore, Md., 1989; Chang et al., Somatic Gene Therapy, CRC Press, Ann Arbor, Mich., 1995; Vega et al., Gene Targeting, CRC Press, Ann Arbor, Mich., 1995; Vectors. A Survey of Molecular Cloning Vectors and Their Uses, Butterworths, Boston, Mass., 1988; and Gilboa et al., BioTechniques 4:504-12, 1986, and include, for example, stable or transient transfection, lipofection, electroporation and infection with recombinant viral vectors.

Recombinant methods known in the art can also be used to achieve the antisense inhibition of a target nucleic acid. For example, vectors containing antisense nucleic acids can be employed to express an antisense message to reduce the expression of the target nucleic acid and therefore its activity.

The present invention also provides a method of evaluating if a compound inhibits transcription or translation of an LDLR gene and thereby modulates (i.e., reduces) the ability of the cell to express LDLR on its surface, comprising transfecting a cell with an expression vector comprising a nucleic acid sequence encoding LDLR, the necessary elements for the transcription or translation of the nucleic acid; administering a test compound; and comparing the level of expression of the LDLR with the level obtained with a control in the absence of the test compound.

Methods for Screening Antiviral Compounds

The present invention provides for screening methods to identify antiviral compounds for the treatment of virus infection, preferably RNA virus infection, preferable positive strand RNA virus infection, preferably flavivirus infection, most preferably HCV infection. The method provides for screening methods to identify antiviral compounds including but not limited to the following types: derivatives of natural LDLR ligands (e.g. APOB and APOE (SEQUENCE:12 and SEQUENCE:13)), antibodies and antibody fragments, small molecules, polypeptides, and proteins.

The invention provides for methods that assess the ability of potential antiviral compounds to bind specifically and with high affinity to the LDLR receptor or polypeptide fragments thereof. As one skilled in the art will recognize, numerous such methods of compound screening are available and well known in the art. In one preferred embodiment fragments of the LDLR protein (e.g. the polypeptides of SEQUENCE:10 or SEQUENCE:11 or portions of SEQUENCE:20) are expressed in E. coli, yeast, baculovirus, or other recombinant protein expression system using vectors constructed from all or part of any one of the nucleic acid sequences of SEQUENCE:1-4. Recombinantly expressed and purified LDLR polypeptides are immobilized on the surface of microtiter plates by any of a number of well known covalent or non-covalent methods. Test antiviral compounds are bound to the protein-coated surface, and the kinetics and thermodynamics of test compound binding measured using any of a number of well known methods in the art. Various techniques are used to measure both specific and non-specific test compound binding as one skilled in the art will recognize.

Test compounds that bind with high affinity and specificity to LDLR are then evaluated for their antiviral properties. In preferred embodiments, the antiviral activity of test compounds are evaluated by their ability to reduce virus titers of a test virus, by reducing virus gene or protein expression during infection, by reducing virus genome nucleic acid levels, or simply by their ability to inhibit virus particle or protein binding to the cell surface or to purified LDLR protein or polypeptide derivatives thereof. As one skilled in the art will recognize, there are numerous methods for assessing the antiviral activity of a test compound, which are dependent on the particular virus and cell culture system used. Methods of measuring antiviral activity include but are not limited to the measurement of: virus replication by Taqman or RT-PCR, virus gene expression by Northern blot, virus protein expression by Western blot, virus particle release into the overlying media, and the cytotoxic effects of virus infection using cytotoxicity assays (e.g. lactate dehydrogenase release) or metabolic assays (e.g. 3-(4,5-dimethylthiazol-2-yl)-2,5-diphenyltetrazolium bromide (MTT) conversion assay). The antiviral effect of test compounds are also measured in whole organisms using numerous metrics and methods available in the art including: virus induced organism death, organ virus titers, tissue histopathology, organ function studies, etc.

As one skilled in the art will recognize, test compounds are preferred that bind specifically and with high affinity to the LDLR receptor complex and inhibit virus infection without inhibiting the normal function of LDLR in cholesterol metabolism. Antiviral test compounds are evaluated for their inhibitory effects on LDL and VLDL binding to the LDLR protein, receptor-mediated endocytosis of LDLR and attached ligands, subcellular trafficking and acidification of LDLR containing endosomes, and ligand release and LDLR receptor recycling to the plasma membrane. As one skilled in the art will recognize, numerous methods are available to measure each step in the normal metabolic pathways mediated by the LDLR. Test compounds are also evaluated for their effects on serum cholesterol levels when administered to animals.

### Preferred Embodiments

Utilizing methods described above and others known in the art, the present invention contemplates a screening method comprising treating, under amplification conditions, a sample of genomic DNA, isolated from a human, with a PCR primer pair for amplifying a region of human genomic DNA containing any of nucleotide (nt) positions 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 of low density lipoprotein receptor (LDLR, Genbank accession no. NT_{—}011295.10, also shown as SEQUENCE:1 in FIG. 1). Amplification conditions include, in an amount effective for DNA synthesis, the presence of PCR buffer and a thermocycling temperature. The PCR product thus produced is assayed for the presence of a mutation at the relevant nucleotide position(s) (as further described by described by any one of SEQUENCE:5-9, SEQUENCE:14-19, and SEQUENCE:45-61). In one embodiment, the PCR product contains a continuous nucleotide sequence Amplicon bound by two PCR primers, PrimerA and PrimerB and containing at least one of the aforementioned mutations. In another embodiment, the Amplicon, PrimerA, and PrimerB as described above in Tables 1 and 2 are exemplary of the PCR products and corresponding primers.

In one preferred embodiment, the PCR product is assayed for the corresponding mutation by treating the amplification product, under hybridization conditions, with an oligonucleotide probe specific for the corresponding mutation, and detecting the formation of any hybridization product. Oligonucleotide hybridization to target nucleic acid is described in U.S. Pat. No. 4,530,901.

The PCR admixture thus formed is subjected to a plurality of PCR thermocycles to produce LDLR and mutant LDLR gene amplification products. The amplification products are then treated, under hybridization conditions, with an oligonucleotide probe specific for each mutation. Any hybridization products are then detected.

In another preferred embodiment, the invention contemplates use of the screening method described above to determine an individual's resistance to viral infection, particularly flavivirus infection, most particularly HCV infection. In this embodiment, the pattern of mutations detected to be present and absent in the individual under consideration are analyzed against known patterns of mutation (or standard) correlated with viral resistance. In particular embodiments, such analysis includes without limitation, the requirement for absolute matching of the individual's pattern of mutation against the known standard for the individual to be determined to be resistant to infection. In other embodiments, statistical methods known to those skilled in the art are used to numerically estimate the individual's degree of resistance to viral infection. Exemplary statistical methods include application of odds ratios or regression coefficients previously determined by statistical modeling such as provided in the preferred modes and examples of the present invention. In one preferred embodiment, the logistic regression coefficients determined for previously determined standard haplotypes are applied to the individual's two haplotypes under the appropriate genetic model. A composite numerical estimate, including without limitation an odds ratio, is then used to estimate the individual's degree of resistance to infection.

The following examples are intended to illustrate but are not to be construed as limiting of the specification and claims in any way.

### EXAMPLES

### Example 1

### Preparation and Preliminary Screening of Genomic DNA

This example relates to screening of DNA from two specific populations of patients, but is equally applicable to other patient groups in which repeated exposure to HCV is documented, wherein the exposure does not result in infection. The example also relates to screening patients who have been exposed to other flaviviruses as discussed above, wherein the exposure did not result in infection.

Here, two populations are studied: (1) a hemophiliac population, chosen with the criteria of moderate to severe hemophilia, and receipt of concentrated clotting factor before January, 1987; and (2) an intravenous drug user population, with a history of injection for over 10 years, and evidence of other risk behaviors such as sharing needles. The study involves exposed but HCV negative patients, and exposed and HCV positive patients.

High molecular weight DNA is extracted from the white blood cells from IV drug users, hemophiliac patients, and other populations at risk of hepatitis C infection, or infection by other flaviviruses. For the initial screening of genomic DNA, blood is collected after informed consent from the patients of the groups described above and anticoagulated with a mixture of 0.14M citric acid, 0.2M trisodium citrate, and 0.22M dextrose. The anticoagulated blood is centrifuged at 800×g for 15 minutes at room temperature and the platelet-rich plasma supernatant is discarded. The pelleted erythrocytes, mononuclear and polynuclear cells are resuspended and diluted with a volume equal to the starting blood volume with chilled 0.14M phosphate buffered saline (PBS), pH 7.4. The peripheral blood white blood cells are recovered from the diluted cell suspension by centrifugation on low endotoxin Ficoll-Hypaque (Sigma Chem. Corp. St. Louis, Mo.) at 400×g for 10 minutes at 18° C. (18° C.). The pelleted white blood cells are then resuspended and used for the source of high molecular weight DNA.

The high molecular weight DNA is purified from the isolated white blood cells using methods well known to one skilled in the art and described by Maniatis, et al., Molecular Cloning: A Laboratory Manual, 2nd ed. Cold Spring Harbor Laboratory, Sections 9.16-9.23, (1989) and U.S. Pat. No. 4,683,195.

Each sample of DNA is then examined for a mutation described by any one of SEQUENCE:5-9, SEQUENCE:14-19, and SEQUENCE:45-61 at the corresponding position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506013, 2506029-2506031, 2506056, or 2506062 with reference to the nucleotides positions of Genbank Accession No. NT_{—}011295.10, corresponding to the low density lipoprotein receptor gene (LDLR, also provided as SEQUENCE:1 in FIG. 1).

### Example 2

### Mutations in LDLR Gene Examined in Study of Resistance to HCV Infection

Using methods described in Example 1, a population of 162 unrelated hemophiliac patients and intravenous drug users was studied by genotyping each subject at sites of mutation in LDLR (as disclosed in any one of SEQUENCE:5-9, SEQUENCE:14-19, and SEQUENCE:45-61). In this study of resistance to HCV infection, the population was grouped into 47 cases that were hepatitis C negative despite extremely high risk of having been infected and 115 controls that were hepatitis C positive. The overall reference allele frequency in this population is given in Table 3 where the reference allele is that found at the corresponding position in SEQUENCE:1 of FIG. 1.

TABLE 3

| | Reference Allele |
|---|---|
| Mutation | Frequency |
| SEQUENCE: 5 | 90.6% |
| SEQUENCE: 6 | 94.5% |
| SEQUENCE: 7 | 61.1% |
| SEQUENCE: 8 | 93.8% |
| SEQUENCE: 9 | 59.6% |
| SEQUENCE: 14 | 40.3% |
| SEQUENCE: 15 | 96.5% |
| SEQUENCE: 16 | 86.5% |
| SEQUENCE: 17 | 85.1% |
| SEQUENCE: 18 | 40.1% |
| SEQUENCE: 19 | 84.7% |
| SEQUENCE: 45 | 59.2% |
| SEQUENCE: 46 | 58.1% |
| SEQUENCE: 47 | 26.0% |
| SEQUENCE: 48 | 10.7% |
| SEQUENCE: 49 | 74.4% |
| SEQUENCE: 50 | 79.9% |
| SEQUENCE: 51 | 51.2% |
| SEQUENCE: 52 | 80.8% |
| SEQUENCE: 53 | 8.5% |
| SEQUENCE: 54 | 75.4% |
| SEQUENCE: 55 | 22.0% |
| SEQUENCE: 56 | 80.7% |
| SEQUENCE: 57 | 29.5% |
| SEQUENCE: 58 | 29.7% |
| SEQUENCE: 59 | 78.8% |
| SEQUENCE: 60 | 19.3% |
| SEQUENCE: 61 | 48.7% |

### Example 3

### Preparation and Sequencing of cDNA

Total cellular RNA is purified from cultured lymphoblasts or fibroblasts from the patients having the hepatitis C resistance phenotype. The purification procedure is performed as described by Chomczynski, et al., Anal. Biochem., 162:156-159 (1987). Briefly, the cells are prepared as described in Example 1. The cells are then homogenized in 10 milliliters (ml) of a denaturing solution containing 4.0M guanidine thiocyanate, 0.1M Tris-HCl at pH 7.5, and 0.1M beta-mercaptoethanol to form a cell lysate. Sodium lauryl sarcosinate is then admixed to a final concentration of 0.5% to the cell lysate after which the admixture was centrifuged at 5000×g for 10 minutes at room temperature. The resultant supernatant containing the total RNA is layered onto a cushion of 5.7M cesium chloride and 0.01M EDTA at pH 7.5 and is pelleted by centrifugation. The resultant RNA pellet is dissolved in a solution of 10 mM Tris-HCl at pH 7.6 and 1 mM EDTA (TE) containing 0.1% sodium docecyl sulfate (SDS). After phenolchloroform extraction and ethanol precipitation, the purified total cellular RNA concentration is estimated by measuring the optical density at 260 nm.

Total RNA prepared above is used as a template for cDNA synthesis using reverse transcriptase for first strand synthesis and PCR with oligonucleotide primers designed so as to amplify the cDNA in two overlapping fragments designated the 5′ and the 3′ fragment. The oligonucleotides used in practicing this invention are synthesized on an Applied Biosystems 381A DNA Synthesizer following the manufacturer's instructions. PCR is conducted using methods known in the art. PCR amplification methods are described in detail in U.S. Pat. Nos. 4,683,192, 4,683,202, 4,800,159, and 4,965,188, and at least in several texts including PCR Technology: Principles and Applications for DNA Amplification, H. Erlich, ed., Stockton Press, New York (1989); and PCR Protocols: A Guide to Methods and Applications, Innis, et al., eds., Academic Press, San Diego, Calif. (1990) and primers as described in Table 1 herein.

The sequences determined directly from the PCR-amplified DNAs from the patients with and without HCV infection, are analyzed. The presence of a mutation in the LDLR gene can be detected in patients who are seronegative for HCV despite repeated exposures to the virus.

### Example 4

### Antisense Inhibition of Target RNA

### A. Preparation of Oligonucleotides for Transfection

A carrier molecule, comprising either a lipitoid or cholesteroid, is prepared for transfection by diluting to 0.5 mM in water, followed by sonication to produce a uniform solution, and filtration through a 0.45 μm PVDF membrane. The lipitoid or cholesteroid is then diluted into an appropriate volume of OptiMEM™ (Gibco/BRL) such that the final concentration would be approximately 1.5-2 nmol lipitoid per μg oligonucleotide.

Antisense and control oligonucleotides are prepared by first diluting to a working concentration of 100 μM in sterile Millipore water, then diluting to 2 μM (approximately 20 mg/mL) in OptiMEM™. The diluted oligonucleotides are then immediately added to the diluted lipitoid and mixed by pipetting up and down.

### B. Transfection

Human PH5CH8 hepatocytes, which are susceptible to HCV infection and supportive of HCV replication, are used (Dansako et al., Virus Res. 97:17-30, 2003; Ikeda et al., Virus Res. 56:157-167, 1998; Noguchi and Hirohashi, In Vitro Cell Dev. Biol Anim. 32:135-137, 1996.) The cells are transfected by adding the oligonucleotide/lipitoid mixture, immediately after mixing, to a final concentration of 300 nM oligonucleotide. The cells are then incubated with the transfection mixture overnight at 37° C., 5% CO₂ and the transfection mixture remains on the cells for 3-4 days.

### C. Total RNA Extraction and Reverse Transcription

Total RNA is extracted from the transfected cells using the RNeasy™ kit (Qiagen Corporation, Chatsworth, Calif.), following protocols provided by the manufacturer. Following extraction, the RNA is reverse-transcribed for use as a PCR template. Generally 0.2-1 μg of total extracted RNA is placed into a sterile microfuge tube, and water is added to bring the total volume to 3 μL. 7 μL of a buffer/enzyme mixture is added to each tube. The buffer/enzyme mixture is prepared by mixing, in the order listed:
4 μL 25 mM MgCl₂
2 μL 10× reaction buffer
8 μL 2.5 mM dNTPs
1 μL MuLV reverse transcriptase (50 u) (Applied Biosystems)
1 μL RNase inhibitor (20 u)
1 μL oligo dT (50 pmol)

The contents of the microfuge tube are mixed by pipetting up and down, and the reaction is incubated for 1 hour at 42° C.

### D. PCR Amplification and Quantification of Target Sequences

Following reverse transcription, target genes are amplified using the Roche Light Cycler™ real-time PCR machine. 20 μL aliquots of PCR amplification mixture are prepared by mixing the following components in the order listed: 2 μL 10×PCR buffer II (containing 10 mM Tris pH 8.3 and 50 mM KCl, Perkin-Elmer, Norwalk, Conn.) 3 mM MgCl₂, 140 μM each dNTP, 0.175 pmol of each LDLR oligo, 1:50,000 dilution of SYBR® Green, 0.25 mg/mL BSA, 1 unit Taq polymerase, and H₂0 to 20 μL. SYBR® Green (Molecular Probes, Eugene, Oreg.) is a dye that fluoresces when bound to double-stranded DNA, allowing the amount of PCR product produced in each reaction to be measured directly. 2 μL of completed reverse transcription reaction is added to each 20 μL aliquot of PCR amplification mixture, and amplification is carried out according to standard protocols.

### Example 5

### Treatment of Cells with LDLR RNAi

Using the methods of Example 5, for antisense treatment, cells are treated with an oligonucleotide based on the LDLR sequence (SEQUENCE:1). Two complementary ribonucleotide monomers with deoxy-TT extensions at the 3′ end are synthesized and annealed. Cells of the PH3CH8 hepatocyte cell line are treated with 50-200 nM RNAi with 1:3 L2 lipitoid. Cells are harvested on day 1, 2, 3 and 4, and analyzed for LDLR protein by Western analysis, as described by Dansako et al., Virus Res. 97:17-30, 2003.

### Example 6

### Haplotype Associated with Resistance to HCV Infection

A subset of case and control Caucasian individuals from the study of Example 2 were selected for further analysis. Subject genotypes for the haplotype spanning the six mutations described by SEQUENCE:8, SEQUENCE:9, SEQUENCE:19, SEQUENCE:46, SEQUENCE:49, and SEQUENCE:51 were analyzed and subject haplotypes inferred by Expectation Maximization (EM) methods. The haplotype defined as GCCTTG for the six defining mutations listed above was found at a frequency of 16.7% in cases and 1.7% in controls, leading to a chi-square value of 13.2 (p=0.00027). Therefore this haplotype is significantly associated with resistance to HCV infection. The six defining mutations span exons 8 through 17 of the LDLR gene exclusively and make up the bulk of the region encoding the EGF precursor homology domain.

### Example 7

### Haplotype Confers Resistance to HCV Infection in Additive Genetic Model

A subset of case and control Caucasian individuals from the study of Example 2 were selected for genetic modelling analysis. Subject genotypes for the haplotype spanning the seven mutations described by SEQUENCE:8, SEQUENCE:9, SEQUENCE:14, SEQUENCE:16, SEQUENCE:19, SEQUENCE:52, and SEQUENCE:54 were analyzed and subject haplotypes inferred by Expectation Maximization (EM) methods. The haplotype defined as GCACCGG for the seven defining mutations listed above was found at an overall frequency of 18.0% in cases and 4.2% in controls. Both inferred parental haplotypes for each case and control subject were analyzed in three genetic models by logistic regression. Of the dominant, additive, and recessive genetic models examined, the additive model produced a significant odds ratio of 3.3 (p=0.04) indicating that this haplotype confers resistance to HCV infection in an additive manner. The mutations defining this haplotype span both the EGF precursor homology domain as well as 3′-UTR of the LDLR gene. As a closely related haplotype (GCACCAA) differing in only the state of the spanned 3′-UTR mutations does not show resistance, this result demonstrates that the genetic makeup of both the EGF precursor homology region and the 3′-UTR contribute to resistance to HCV infection.

### Example 8

### Haplotype Associated with Susceptibility (Decreased Resistance) to HCV Infection

A subset of case and control Caucasian individuals from the study of Example 2 were selected for further analysis. Subject genotypes for the haplotype spanning the seven mutations described by SEQUENCE:5, SEQUENCE:8, SEQUENCE:14, SEQUENCE:16, SEQUENCE:19, SEQUENCE:47, and SEQUENCE:48 were analyzed and subject haplotypes inferred by Expectation Maximization (EM) methods. The haplotype defined as TGGCCGG for the seven defining mutations listed above was found at a frequency of 2.7% in cases and 10.4% in controls, leading to a chi-square value of 3.9 (p=0.05). Therefore this haplotype is significantly associated with susceptibility (decreased resistance) to HCV infection. The seven defining mutations span both the R1 ligand-binding domain and exons 8 through 17 of the LDLR gene. The power of the R1 ligand-binding domain mutation SEQUENCE:5 in this analysis indicates strong contribution of this region to resistance to HCV infection in addition to that observed for the EGF precursor homology domain region.

The foregoing specification, including the specific embodiments and examples, is intended to be illustrative of the present invention and is not to be taken as limiting. Numerous other variations and modifications can be effected without departing from the true spirit and scope of the invention. All patents, patent publications, and non-patent publications cited are incorporated by reference herein.

1. (canceled)

2. A human genetic screening method for determining said human's resistance to virus infection comprising: i) detecting in a nucleic acid sample the presence or absence of at least one LDLR mutation selected from the group consisting of: substitution of a non-reference nucleotide for a reference nucleotide at nucleotide position 2473714, 2473879, 2484259, 2485102, 2486983, 2487067, 2489602, 2489746, 2490268, 2490282, 2490356, 2490404, 2493683, 2496743, 2501350, 2501609, 2504679, 2504717, 2504846, 2505109, 2505298, 2505460, 2505567, 2506011, 2506056, and 2506062; a base insertion at nucleotide position 2506013; or a three-base deletion at nucleotide position 2506029-2506031, wherein said nucleotide position is with reference to SEQ ID NO:1 and ii) analyzing the pattern of presence or absence of each evaluated mutation with a previously determined standard for evaluating resistance to virus infection, thereby determining said human's resistance to virus infection.

3. The method of claim 2 wherein detecting in a nucleic acid sample the presence or absence of at least one LDLR mutation is performed by a technological method selected from the group consisting of the polymerase chain reaction (PCR) or other thermal cycler-based DNA synthetic techniques, molecular cloning in a plasmid or other suitable vector, denaturing gradient gel electrophoresis, detection of length variants in a DNA sample by agarose or polyacrylamide gel electrophoresis, gel or capillary electrophoresis and analysis of products tagged with a fluorescent or other label incorporated into the DNA, DNA sequence determination, hybridization of two complementary nucleic acid molecules, oligonucleotide ligation assay, mass spectroscopic analysis and any heteroduplex-based or similar methods for detecting base mismatches or length variants.

4. (canceled)

5. The method of claim 2 wherein the genetic screening method includes the use of at least one polynucleotide selected from the group consisting of 5, 10, 15, 20 or more consecutive nucleotides of any one of SEQ ID NO:1, SEQ ID NO:2, SEQ ID NO:3, and SEQ ID NO:4 and 5, 10, 15, 20 or more consecutive nucleotides of the complement of any one of SEQ ID NO:1, SEQ ID NO:2, SEQ ID NO:3, and SEQ ID NO:4.

6. The method of claim 5 wherein the polynucleotide is covalently or noncovalently attached to a fluorescent molecule, radioisotope, or heavy isotope.

7-8. (canceled)

9. The method of claim 5 wherein the polynucleotide is covalently or noncovalently attached to a solid support.

10-12. (canceled)

13. A method of treating virus infection in a mammal comprising administering to a mammal in need of such treatment an inhibitor that hybridizes to 5, 10, 15, 20 or more consecutive base pairs of the nucleic acid of at least one of SEQ ID NO:1-4 or their complements.

14. The method of claim 13 wherein the virus is an RNA virus.

15. The method of claim 13 wherein the virus is a positive strand RNA virus.

16. The method of claim 13 wherein the virus is a flavivirus.

17. The method of claim 13 wherein the virus is the hepatitis C virus.

18. The method of claim 12 wherein the inhibitor is a small molecule.

19. The method of claim 12 wherein the inhibitor is an antibody or antibody fragment.

20. The method of claim 19 wherein the antibody or antibody fragment is developed using 5, 10, 15, 20 or more consecutive amino acids of SEQ ID NO:10, SEQ ID NO:11, or SEQ ID NO:20 as an antigen or immunogen.

21-47. (canceled)

48. A method of identifying antiviral drugs comprising assaying candidates for their ability to inhibit binding of the HCV nucleocapsid or associated proteins to 5, 10, 15, 20 or more consecutive amino acids of the polypeptide of SEQ ID NO:10, SEQ ID NO:11, or SEQ ID NO:20.

49. The method of claim 48 wherein the candidates do not inhibit the binding, internalization or metabolism of low density lipoprotein (LDL) and very low density lipoprotein (VLDL) via the low density lipoprotein receptor.

50. The method of claim 2 wherein the virus is a flavivirus.

51. The method of claim 2 wherein the virus is hepatitis C virus.

52. (canceled)

53. The method of claim 2 wherein analyzing the pattern comprises numerically estimating said human's degree of resistance to said virus infection by applying the statistical model and parameters determined in the process of identifying the standard of resistance and thereby determining said human's resistance to said virus infection.

54. The method of claim 53 wherein the statistical model is a genetic model.

55. The method of claim 53 wherein the standard of resistance is a haplotype statistically associated with resistance to said virus infection.

56. (canceled)


SEQUENCE LISTING (63 sequences)

SEQ ID NO: 1 - DNA, 49020 bp, Homo sapiens
tccgcctcctgggttcatgccattctcctgcctctgcctcatgagtaactgagactacaggcgcccaccaccacgcccggctaatttttttgtatttttttagtagagatggggtttcaccttgttagccaggatggtctcgatctcctgacctcgtgatccacccgtctcggcctcccaaaatgctggcattacaggcgtgagccaccgcacccagccttaaatttttttttaagggaaatcaaacccagtgatattgggccagtacagtggctcacacctgtaattccaccactttgggaggctgaggcaggtgaatcacctgaggtcaggagttcgagaccagcccggcaaacatggcgaaaccccgtctctactaaaaataagaaaattagccgggcgtagtggcatgcacctgtaatctcagctactcgggaagctgaggcatgagaatcgcttgaacctgggagcaggatgttgcagtgaaccgatatcacaccactgcactccagcctgggtgacagagcaagactctgtctcaaaaaaaaaaagaaaaaaaaatccagtgatacttactttttaaatttttatttacttattttttgctttaagttgaatctttaaacttatctttatttttgagacacagtctcactctgtcgcccaggctggagtgcagtggtacaaccacagctcagtgcagcgttgacctcctgggctcaagccatcctcccgcctcagcctcccgagtagctgggactacaggcgcacacaaccatgtccagcttatttttgtattttttgtagagacagggtcccactgtgttgccctggcttgttctgaactcctaggctcaagtgatccccccgcctcaccctcccaaagtgctgggattacaggcatgagccaccacatccagacttcacttttttgtttaatgtcgcaaatggcataaggaatgggattcaatggggacacatttataaacgttgcagcagctcctagaacttgcctatccttgtaaacttctctaggtgattgctaattacttctttttttttttttttttttgagacggagtctcactctgtcgcccaggctggagtacagtggcgcaatctcgtctcactgcaaactccacctcccgggttcacgccattctcctgcctcagcctcccgagtagctgggactacaggcacccgccaccacgcccggctaattttttgtatttttttttagtagaggtggggtttcactgtgttatccaggatggtcttgatctcctgacctcgtgatccacctgcctcagcctcccaaagtgctgggattacaggcgtgagccaccatgcccagcccgctaattatttcaatttgaccttgacactgagcctgccaagtaggttcaagcattttgatggcccctttacaggttgggaaagctaatttatctgtccaaggccgaattctgaaactgagtcttaactgccaaaaattcttatcatcaatttcttcttctgggttgggcacagtggctcatgcctgtaaagccagcaatttgagaggcatcatgatgcaagaggaagaggattgagtgaagctaggagtttgggaccagcctgggcaacatagtgagaccccatctataaaaaaaaattaaaaattagttgggcatggtggtgcactcctgtggtcctagctattcaggaggctgaggtgggaggattccttgagcccagggttgacgctgcagagagctgtgatcacgccactgcagtccagcctgagtgacagctggaaataatgataaataaataataaataattatttaaaaaattataataaaaataattaaaaaattattttccctgattaatctttttttttgtccttctgagagttcaatttgtcccttttctgcctggtctcctaggtttccctaaaatcctgctgagaggttagcactgcctgccaaagtcagtttgcaaaatcccagagaaatccagcttattcctgggggaaccgccaagactgcccagccctgtgtggggttcaggcaagtttctcacatgtgcctttttggcaagaggcctctggcaaccccatgagtccccaaagagactcaattctaaaagttggtctccaccagctctctgtggcttaggggttcaagttcaactgtgaaagccctgttttgttttgattttgctttgagggagaggaaaccgcccttctgtttgttcaactccttctcctaaggggagaaatcaatatttacgtccagactccaggtatccgtacaattgatttttcagatgtttatactcagccaaaggcgggatcccacaaaacaaaaaatatttttttggctgtacttttgtgaagattttatttaaattcctgattgatcagtgtctattaggtgatttggaataacaatgtaaaaacaatatacaacgaaaggaagctaaaaatctatacacaattcctagaaaggaaaaggcaaatatagaaagtggcggaagttcccaacatttttagtgttttccttttgaggcagagaggacaatggcattaggctattggaggatcttgaaaggctgttgttatccttctgtggacaacaacagcaaaatgttaacagttaaacatcgagaaatttcaggaggatctttcagaagatgcgtttccaattttgagggggcgtcagctcttcaccggagacccaaatacaacaaatcaagtcgcctgccctggcgacactttcgaaggactggagtgggaatcagagcttcacgggttaaaaagccgatgtcacatcggccgttcgaaactcctcctcttgcagtgaggtgaagacatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcaggtaaggcttgctccaggcgccagaataggttgagagggagcccccggggggcccttgggaatttatttttttgggtacaaataatcactccatccctgggagacttgtggggtaatggcacggggtccttcccaaacggctggagggggcgctggaggggggcgctgaggggagcgcgagggtcgggaggagtctgagggatttaagggaaacggggcaccgctgtcccccaagtctccacagggtgagggaccgcatcttctttgagacggagtctagctctgtcgcccaggatggagtgcagtggcacgatctcagctcactgcaacctccgcctcccgggtttaagcgagtctcctctctcagcctcccgaatagctgggattacaggcgcccaaccaccacgcccgcctaatttttgtatttttagtagagacgggttttcaccattttggccaggctggtctcgaaccccgacctcaggtgatctgcccaaaagtgctgggattacaggcgtcagccaccgcgcccggccgggaccctctcttctaactcggagctgggtgtggggacctccagtcctaaaacaagggatcactcccacccccgccttaagtccttctgggggcgagggcgactggagacccggatgtccagcctggaggtcaccgcgggctcaggggtcccgatccgctttgcgcgaccccagggcgccactgccatcctgagttgggtgcagtcccgggattccgccgcgtgctccgggacgggggccaccccctcccgcccctgcccccgcccctttggcccgccccccgaattccattgggtgtagtccaacaggccaccctcgagccactccccttgtccaatgtgaggcggtggaggcggaggcgggcgtcgggaggacggggcttgtgtacgagcggggcggggctggcgcggaagtctgagcctcaccttgtccggggcgaggcggatgcaggggaggcctggcgttcctccgcggttcctgtcacaaaggcgacgacaagtcccgggtccccggagccgcctccgcgacatacacgagtcgccctccgttatcctgggccctcctggcgaagtccccggtttccgctgtgctctgtggcgacacctccgtccccaccttgtcctggggggcgccctcgccccaccagccccgatcaagttcacagaggggcccccggccaccctcaaggcctcggttccttacgaggttgaaacgttgcctcagaatctccccgcccctccttggtctgcagccgagatcttcagccacggtggggcagctatcccccgggaccgaccccctggggtggcctcgcttcttcagaggctgtgaatggcttcggttcagctgtccaagcggcgatttttcctctgggtgaaatggattagattttagatttccacaagaggctggttagtgcatgatcctgagttagagctttttaggtggctttaaattagttgcagagagacagcctcgccctagacaacagctacatggccctttccctcctgagaaccagcctagcctagaaaaggattgggattgcctgatgaacacaaggattgcaggaaactttttttttaattggcaagggggttggctttgactggatggagagctttgaactgccttgaaattcacgctgtaactaacacaccagtttcctctgggaggccagagagggagggagggtgtaatgaaatacggatgattgttcttttatttttatttacttatttattttttaactttttgtagagatgaggtctcgcttggttgctcaggctggtcttgaactcctggcctcaagcgatcctcctacctcagcctcccaaagtgttgggattacaggagtgagccaccgcgccccaccggggatgatgatgattgcaaacattctgccactcagttttacaaaagaaagagaggcactggattaatgtgtatctcactcaccaatcaacctcttccttaagagaaaatgttaaggaagtcttaggcaaggccttgtttgttcatcactttagtttctctctcccgggatggctgagaatgtgatgtttcctctgttgtcaaggagactacacccctgatgttttcctccagacttctgagagctggtgtgtgtttctagcactttctagctgcaccacctcacgctgtagctggcttcaaggcatatccaggggggagtttcttgtccatttcctttacaaagggaagttgttggaatctgaaccgcaagccttcacttagaccaaaatcaggcaacagcggtgagcgcagctccaaacgtgtcaatgactcacccaaatttgagtaagggagttggctgctttaacgagccgcagggtgattcccttgtcatttccggaaatacctatcttccagggaacactgggaaaaaacagggagacctttgttgagacagaaaacctgtaggggaattctgttcctcattcctgctcttatctgtagacttcctccctgataagatccaattctagatgggtcggttgctccttgctttgatgggtgctttgatgggctttattattattattattattattattattattttgatgggctttttgatgtcccttttccttccacactctgtcccaactgtcaagcaaatagccttttgttgctaagagactgcagatgtaaccgaccagcagcaaacagtgagtcaggctctctcttccggaagcaaaatcaattgctgagatcactctggggaaaatacccaccttatttggaaagaagcactgatcaattgatgtctattttttttttttttgagttggagtctcgccctgtcacccaggctggagtgcaatggcataatctcgcctcactgcaatccccgcctcccgggttccagcaattctcctgcctcagcctcctgagtagctggaattataggcgcctgccacaacacccggctaatttttgtatttgtagtagagatggggtttcaccacgttggccaggctggtctcgaactcctgacctcgtgatccacccgcctcagcctcccaaagtccaaggattgcaggcgtgacccactgtgccagccaatcaattgatttctcattcattttcagctggctctgttcccttaagccaggggattttcgtttgtttgtttccccttcaaggaaatgattctagctacagttttgatttccttgtacaactgttttcagtagcacagggaaagaaaacatcgaaagcattcaccacctcatttgtgtgctgggggaaaaagcagaaatgtgtattctctttttttgtttcgatgaccttgttcctgacttgttactcgtgacttgagagatcagagggctagaggactagaatttatagaggtgttttttttgtttgtttatttttgttcgagttgcccaggctggagtgcagtggcgcaatctcggctcactgcaacctctgcctcccaggttcaagcgattcttcggcctcagcctcctgagtagctggaactacaggcgcccgccaccacacccagctaatttttgtatttttcagtagagatgggatttcaccatattggtcaagctggcctcgaactcctgacctcgtgatccacccgcctcagtttcccaaagtgctgggagtacaggcgtgagccgccgtgcccggcctttttgtgtttttgtgtttttgagaggagctcattgctttttaggcttccctagcgtgagaaaatctggggatccatgctctagtttacttcctttttttttttttttttgagatggagtctcgcttagattgcctaatctcagctcattgcaacttctgcctccggggttcaagggattctcgtgtctcagcctcctgggtagctaggatacgggcacccgctaccatgcctggctaattttgtacttttagtagagacagggtttcgccacgttggccaggctggtctcgaactcctgacctcaggtgagccgcctgccttggcctcccaaagtgctgagattacaggcgtgagccaccgcgcttggcctaatttgcttttcctgaaattcaaatggtctaatatgaaaaacgccaaccttgcttgaaagaataagaaagaggtgcggtttcgttgggccgttgatgtttggaacaggactggttttgtccccttgctcggaaagggcagcaactgtgaggacagctccctgacgtgctctcactcagcactgttccgttcctgagcactgtccccactagctaggccaagggagctcatttggcaggcaactgctgtctggctgcgcctgtggcagtaaaatctgcctttattttttggaggcagggtcttgccctgtcgctcaggctgaagtgtgcagttatagctcactgcagcctccagcttctgtactcaactgatcctcctctctcagcctcctgagtagctgggactatacgcacgtgttaccactcccacctcagtttgtttgtttatttatttatttatttatttattgagatggagttttgctcttgctgcccaggctggagtgcaatggcgcgatctcggctcaccgcaacctccacctcctggttcaagcgattctcctgcctcagcctcctgagtagctgggattacaggcatgcaccaccacgcccggctaattttgtatttttcgtagagatggggtttctccacattggttcaggctgttctcgaactcccaacctcaggtgatccacccgcctcagcctcccaaagtgctgggattataggcgtgagcccccgaacccggccactcccagctaagtttaaattttttgtttgtttgttcgtttgtttttattttttgagacagagtctcccgcccaggctggagcgcagatcactgcatccttgacctcccaggcttaagccatcctccccactcagcctcccaagtagctgggattacaggtgtgtgccactatgcttggctaagttgtgtattttttgtagagatggggttcaagggattctcgctttgttgcctcggttggtctcaaactcctgggctcaagcagtcctccctcctcagcctcccaaggtgctggggaaatccacttttgaaacattgtctggagagttgcccaggtggtagatcacagaaataggtcatcgtggggtccttcccatgggtgcagtcttgagccacctgtggccagcaaatatttggagaataatagtcaggggagagcttgaggtccagggaaaggttttgtttttcttcagggaaaggtttttattgttctttatccctccttaaaggaccttcaggtgttactgacattcccggtctacccagtggcacatttagtttgtaagctgggccctcgtacagaggtagggaggtgagagcattggattagtggtcaccaaagctgcggtcacctagtggggtgatcagaggctcctcccttaagatcttgattgccaacgcctctggcccaactttcctttttatttatcgcaagcctcctggaatctcaattgctttttgcccacccggtgtgtcagcacaagaaatgagtcatttcctcctttaagcacagttgaaattgagctgtgagtcagtgaggtgtgtacgatattgtcaaagcggggtgtgtacagtattgacagatctgtagttgggcaagagaattatcagagtttgtgaccacagcagattccaaagctcgactcattttcttctctcttccttcccttttttcttttctttttttttttttttttgacagagtctcgctctgttgcccaggctggagtgcagtggcacaatctgggctcactgcagcccctgcctcctgggttcaaatgattctcatgtttcagcctcccgagtagctgcaattacaggcattcgggttcaagtgattctcctgcctcagccacctgagcagctgggattacaggcgcccgccaccacgcccggctaatttttgtatttttagtagagacggggtttcaccatgttggccaggctggtctcgaactcctgaactcaggtgatccgcccacttcggcctcccaaagtgctgagattacagacgtgagtcaccgcgcccagcctgttctgttctttaattctcaaaacaccctctaggaagtagagactgccattctcccccattttacagatcaggaaactgagtcccagaaggatttagtcagttacccaagttgttctagttaaatggcctggaaagccagtgaagcccaggattgtctatctaacccccttactactctaactttcagggaatccacatgaatgtgctgggtcaaccatcaaagttgaaatggataaagggggctggatgcggtggctgatgcctgtaatcctagcactttgggaggccgagatgggtgggtggattgcttgagcccaagagtttgagaccagcctgggcaacatagtgagacacctgtctctgcaaaaaataaataaaaagttagctgagtgtgatggtgcacccctctagtcacagctgttgagttaggcttaggcaggaggatcgcatgaacctgggaggtggaggcggccgtgagcctcagtcatgccactgcactccaacctgggcaacagagtgaaagccggtgtccgaaagagaaagaaaaaaagacatagatacatcttttaaagttaggttgtatgttaattacctacaactcagtttcaactgtgcttaaaggaggaaatgactcatttcttgctacatatcaaattagcccaaaatgtagtggcttaaaacaacacatttatgatttctcagtttttgcgtgtcaggaatttggaagcagcacagctagacggttccagctcagggtctctcatgaagttgcaatcaaaatattggcaggagagaaaaacatattttcagaagctgcaggcataggaagacttggctggggttgaaggatccacttccaagatggcgcactcagtggctcttggctggaggcctcagttccctgctgcgtggagctctccctccagctgcttgagtggactcatgacatgcagctggcctcccctggagcagtcgatccaacaatgagcatggccatgaactaggctcagaagccactccctgtcgtctctacattttcctatcagaagcaagtcattaaaagtccagtgccactccaggggagacgaattaggctctgccttctgaaaggattatcacagaagatgcggtcctatattctttttttaaaattattcttttttttattttgtagagatggggtcttggtatgttgcctaggccagtctggaattcctgggctcaaacaatcctgtctctgcctcccaaagtgttgggattacaggcatgagccactgcacctggtcatgtggtcatattttctttttcttttttttttttttttgagacagagtctctgtcgcccaggctggagtatggtggcgtgatctcagttcactgcagcctccgcctcccgggttcaagcgattctcctgcctcagcctcctgagtagctgggattacaggcgcccgccaacatgcccagctaatttttttagtagagatggggtttcaccatgttagccaggatggtctcgatctcctgatttggtgatccgcccaccttggcctcccaaagtttcaaccatcgatcagaacttattgatgtacttatgtagctaggcacggtggcgcgtgcctgtaatcccagctacttggaagggttaaggcaggagaatcgcttgaacctgggaggcagaggttacagtgagtcaagatcataccattgcactccagtctgggcaacagaatgagactctgtctcaaaaacaaaaaacaaacccttgtatgtgattttcctggatagcatctgttacatcttcacaaagataaaaagtcagacttggctgggcatggtggctcacacctgtaatcccagcactgagaggctgaggcaggcagatcacttgaggtcaggaatttgagaccaggctgggcagcatggtgaaaccccgtctctacaaaaaatacaaaaattagccgggtgtggtgtcacgcacctgtattcccaagctactcaggaagctaaggcaggagaatcacttgaacccagaggtggaggtttgcagtgagttgagattgtgccattgcactccagcctgggcgacagagtgagactctgtgtcaaaaataaaataaaataaaattttaaaaaaggcagatttttttttcttcttggtattgttaccttattatagtaataataagtgcatagtgcatgctgagataagcaatcataatttgttattgcggccgggcatggtggctccagcctataatcccagcactttggtcaggagttcaaggccagcctggccaatatagtgaaactccatctctactaaaatacaagaaattacctgggcatggtggcagttgctggtgatccccagctacttgggaggctgaggcaggagaatcgcttgaacctgggaagcagaggttgcagtgagccaagattgcaccactgcactccagcctgggtgacagagtgagactctgtctgaaaataataataataataatttgttattgcttttattgccttagtttacatagggaatcaaagtttatactttgatttataaaagttgctttgattctagttcacagaaccagaatctttcatataaaggtattagagggcccagtgtggtggctcatgcctgtaatcccagcatattgggaggctgaggagggaggatcactttaggagtttgaggccagcctaggcaacatagtgagaccttgtctctacaaaaaattccaacattagctgggcatggtggcatgtgcctgtagtcccatttatttggggggctgaggcaggaggatcacttgagcccacgaggttcaatccaggttgcagtaagccatgatcctgccactgcactccagtttgggtaacagagcgaagctatgtctcaaaaaaagaaaaaaaaagtattctaaatccaaatttaatatataaaactaaatgcaggccaagtgtggtggcatatacctataatcacaacactttgggaggctgaggtgggaggattgcttgagcccaagagttcaagaccagcctaggtaacacagtaagaccccatctctacaaaaagtagaaaaattagcctggcatggtggtgagtgcttttaatcccaactacttagggggctgagatgggaagattgcttgagcctcagagtttgaggctgcagtgggccgtgatcgctccactgatcgctctaaagtgagaccctgtctcaaaaaaaaagaaaatagaagaaaactaaatacattcaataagactttgatctcttttccaaggtgtaaatatattttgggaaattttccagttactttgttctcattttaatgtaataatctaagtcttggttttctaaggaaaagttttctcttattatatcttttgttaatgtttctctcccatttcttttgatctgatcttcagatacatgattatcttcactgctaaatttgtgttctctggcctctacatttataatttctcataattctttatctaagtatttcttccctacctactgaagaaaactcaagttttcttccaccttaatgattatgctgtgtctgtgagttttcttcatgactctttacagtacaagttttttgtttttgtttttttaatggtcagatggatagaacaacacaggttttgtttgttttgttttaacttttaaaaaaattataatagataaagggtctcactacgttgtccaggctgatctcatactcctgggctcaagcaatccacccacctctgcctcccaaagtgctgggattacagtcatgagccaacatgcctgggcagtacaggttttttttgagacggagttttgttcttgttgccgaggctggagtgcaatggcacaatcttggctcaccacaaagtctgcctcccaggttcaagtgattctcctgcctcagcctcctgagtagctgggattacaggcatgtgccaccacgcccagctaattttgtatttttagtagagacggggtttcaccatgttggccaggctggtttcgaactgctgacctcaggtgatctgcccacctcggcctcccaaagtgctgggattacaggcatgagccaccatgcccagctgtagtacaggttttaatatgctaaatactcttcctttctttattaatgtgcatggaagttctaatatttttttcccataccccagagagtccatattttggaatcaacaacactagcctttgttgacaagtgtctctcttgggttccttctttgtgtcctccactgaattttggggttcataaaatttcatttgttgtgcttgcttaattccctgggaatcagactgttcctgatcggatgacatttctggttaattctttagttggcaggaaatagacacaggaaacgtggtcagtttctgattctggcgttgagagaccctttctccttttcctctctctcagtgggcgacagatgcgaaagaaacgagttccagtgccaagacgggaaatgcatctcctacaagtgggtctgcgatggcagcgctgagtgccaggatggctctgatgagtcccaggagacgtgctgtgagtcccctttgggcatgatatgcatttatttttgtaatagagacagggtctcgccatgttggccaggctggtcttgaatttctggtctcaagtgatccgctggcctcggcctcccaaagtgctgggattacaggcaccacgcctggcctgtgacacgattcttaacccctttttgatgatggcggctggaaaagtggccagtggattttgatgtattcaatcatgaattaggaggtggggagagaatgaattattggagctttccttaaagccattaaatggctctattgttttttcaattgatgtgaatttcacataacatgaaattaaccagctcagtggcattaatacatctgcaatgctgtgtggccaccacctctatcttgttccaaaactttgcataacctaatgtctttttttttttttttttttgagacggagtctcgttccatcacccaggctggagtgcagtggtgtgatctcagctcactgcaacctccgcctcccaggttcacgccatcctcctgcctcagcctcccgagtagctgggactacaggcaccctccaccacatccggctaattttttgtatctttagtagagatggggtttcaccatgttagccgggatggtctcgatctcctgacctcgtgatccacctgcctccgcctcccaaagtgctggcattacaggcgtgagccaccatgcccggcctatttttttttttaagagatggagtctaattctgttgcccaggctggagtccagtggtaccatcatacttcactgcagccttgacctcttgggctcaagtgattctcttgcctcgaactcccaaagtattgggattacaggtgtgagccaccgcactcagcctaatgtccagtttttaacaagctccatttaaatgccctccgttttgacccataaaggggtaggcttggccgggcacaatggcttgtgtctgtagtcccagctacttgggaggctgaggcagaaaggcagaaagattgctttataaagcccaggagtttgagggccacctgggtggcatagctagacctcatctctaaaaaataagtaataaataaatatttgtttttgtttttttctttttcttttcttttttttttttttttgagacggagtcttgctctgttgcccaggctggagtgcagtggcgcgatctcagctcactgcaagctgtgcctcctgggttcatgccattctcctgcctcagcctcccgagtagctgggactacaggcgcccactaccacgcccagctaattttttgtatttttagtagagatggggtttcaccacgttagccaggatggtctcaatctcctgacctcgtgatccgccagctttggcctcccaaagtgttgggattacaggcgtgagccactgagcccgccccatatgtatgtatatatatatttttttaaaatgggagaccaggcatggtggctcatgcctagaatcccagcactttgggaagctgaggtaggcggatcacttgaggccatgagtttgagaccagcctgctcaacatgatgaaacttctatctctactaaaaaaaaaagtgggattaggtcaggcacggtggctcacacctgtaatcccagcactttcagaggccgaggcaggaggatcatgaggtcaggagatcgagaccatcctggctaacacggtgaaaccccgtctctactaaaaaaatacaaaaaattagccaggcgtggtggcgggtgcctgtagtcccagctactcaggaggctgaggcaggagaatggcgtgaacccgggaggcggagcttgcagtgagccaagatcgtgccactgtactccagcctgggcgacagagcaagactctgtctcaaaaaaaaaaaaaaaagtgggattgacattctcttcaaagttctggggttttcctttgcaaagacaggattggcaaggccagtgggtcttttttgtgtgtgtgtgtgtgacggagtctcactctgccacccaggctggagtgcaatggcaggatctcggctcaccgcaacctcctcctcccaggttaaagtgattctcctgcctcagcctcccgagtagctgggactacaggtgcccgccaccacacccaactaatttttgtatttttagtagagacagggtttcactatattggccaggctggtcttgaacccctgacctcacgtgatccacccgccttggcctcccaaagtgctgggattacaggcgtgagccactgtgctcggcctcagtgggtctttcctttgagtgacagttcaatcctgtctcttctgtagtgtctgtcacctgcaaatccggggacttcagctgtgggggccgtgtcaaccgctgcattcctcagttctggaggtgcgatggccaagtggactgcgacaacggctcagacgagcaaggctgtcgtaagtgtggccctgcctttgctattgagcctatctgagtcctggggagtggtctgactttgtctctacggggtcctgctcgagctgcaaggcagctgccccgaactgggctccatctcttgggggctcataccaagcctcttccgcccttcaaatccccccttgaccaggaggcattacaaagtggggatggtgctacctcttcgggtttgtcacgcacagtcagggaggctgtccctgccgagggctagccacctggcacacacactggcaagccgctgtgattcccgctggtcgtgatccccgtgatcctgtgatccccgccccgtgaggctgaacacatagtgacgcttgctagccaagcctcaatgacccacgtaacatgaagggggaaaagccagaaagttctgccaaggagcaaggccaagaatcccgaagggaaatggactttgaagctgggcgtcttcttggctgtcttaatacaagtggcacatccaaatccaaaaccccgaaattcaaagtcttgagcacccgaaattctgaaacgtcttgagcactgacctttagaaggaaatgcttattggagcattttggatttcggatttttaccactgagtgtggagtcctaattaggaaaaaaaccaggctgaccgaaccaaaggaaagcaataaaagaaggcagatagggtcaggcacggtggctcacccctgtaatcccagccttttgagaggctgaggcgggtggatcacttgaggtcaggagttcgagagcagcctggccaacacggtgaaaccccatctctactgaaaatacaaaaactagccaggtatggtggcgtctgcctgtaatcccagctactcgggaggctgagacaggagaatcacttgaacctgggaggcagaggttgcagtgagccaatatcacgccattgcactccagcctgggggacaagagcgaaattctgtctcaaaaaaaaagaagaagaaggccgacaaactatgtaactctgcctttctccatggtccagaacacacagccctcctgcgtaaataactccttatcttcctgctcccagctatcatcagacacctcggctgatagaaaattgcaagttagctcactgcaacctcggcattataagtactgcacaaagccctcttcagcgcacagcacaagcaccattctataaaatctccagcaagcggccaggtgcagtggctcatacctgtaatcccagcattttgggagactgaggcgggcggatcacctgaggtcaggagtttgagaccagcctggccaacatggtgaaaccccgtctctattaaaaatacaaaaaaattagccaggcgtggtggcaggtgcctgtaatcccagctacttggaaggctgaggcaggagaatcgcttgaacccgggaggtggaagttgcagtgagccgagatcttgccatcgcactccagcctgggggacaagagtgagacttcgtctcaaaaaaaaaaaaaaaaattcccagcaagcctttgtcttctggcagtcagctcctctcttgctgacctgctcattgctttcttgcaaggtattttcctacctactttctggaataaatctgtctttctgtacttacaactaccttttttaaaatttctttcttttttgagatggagtctcactctgtttgcccaggctggagttcagtggtgcaatctcagctcactgcaacctctacctactgggttcaagcgattctcctgcctcagcttcccgagtagctgggattacaggcgtgcaccagcacgcaggctaatttttgtatttttagtagagacggggtttcaccatgttggccaaggtggtcttgaactcctgacctcaagtgatcctcccacctcagcctcccaaagcgctaggattacggccatgagccactgaggccggctgcacctacaactgtcttgataaattcttacccccacaccactggtccagatagtcagtgctcacccacaacattaaggatattccaaatttgaaacattccaaaatcagaaaaatattccaactctgaaaatattccaaaatccaaaaaaattcaaaatccaaaacacttctggtcccaagcattttagagaagggatactcaacccaaaataaggacagcaattctataaattgtgctaccatcttgcaggtctcagtttaacagctttacacctattagcgcaccagtgctcatagcagtgctgggaaatgtgtacagatgaggaaactgaggcaccgagagggcagtggttcagagtccatggcccctgactgctccccagcccgcctttccaggggcctggcctcactgcggcagcgtccccggctatagaatgggctggtgttgggagacttcacacggtgatggtggtctcggcccatccatccctgcagcccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcggtatgggcggggccagggtgggggcggggcgtcctatcacctgtccctgggctcccccaggtgtgggacatgcagtgatttaggtgccgaagtggatttccaacaacatgccaagaaagtattcccatttcatgtttgtttcttttttttcttttctttctttattttgtttttgagatggagtctcactctgtgatttttttcatctctaaatttcctacatccatatggccaccatgaggccccaggctggccgatggttgctgttagcttattgggaaatcactgtttggaaggtgctggttgttttttgttgtttgttgtttttgtttttgtttttgttttgagacggagtctcgctctgtcgccagggtggagtgcagtggcgcgatcagctcactgcaacctccgcttcctgggttcaagccattctcctgcctcagcctcccaagtagcgcggattacaggcatgtgccaccacctccggctatttttttttctatttagtagagatggggtttcaccatgttagtcaggctggtcatgaactcttgacctcaggtgatccacccgcctcggcctcccaaagtgctgggattacaggcgtgcactgctgcacccagcctttttttgtttttttgagacagggtcttgctgtcacccaggttgaagtaaggtggcacgattatggctcactgcggccttgatctccttggctcaagcgatcctctcacttcagcctctcaagcagttggaaccacaggctgtaccaccaagcctggccaatttttttgtacagacacaggctggtcttgaactcctgggctcaagcaatcctcctgccttggcctcccaaagtgctgggattccaggcatgagccgctgcacccggcaaaaggccctgcttctttttctctggttgtctcttcttgagaaaatcaacacactctgtcctgttttccagctgtggccacctgtcgccctgacgaattccagtgctctgatggaaactgcatccatggcagccggcagtgtgaccgggaatatgactgcaaggacatgagcgatgaagttggctgcgttaatggtgagcgctggccatctggttttccatcccccattctctgtgccttgctgcttgcaaatgatttgtgaagccagagggcgcttccctggtcagctctgcaccagctgtgcgtctgtgggcaagtgacttgacttctcagagcctcacttccttttgttttgagacggagtctcgctctgacacccaggctggagtgctgtggcacaatcacagctcacggcagcctctgcctctgatgtccagtgattctcctgcctcagcctcccgagtagctgagattaaaggcgtataccaccacgcccggctaattttttgtatttttattagagacagggtttctccatgttggccaggctggtcttgaactcctggtctcaggtgatccacccgcctcggcctcccaaagtgctaggattacaggtgtgagccactgcgccaggcctaatttttttgtatttttagtagagatgcggttttgccatattgcccaggctggtctcgaactcctgggctcaagcgatctgcctgccttggcctcccaaagtgctgggattacaggcacaaaccaccgtgcccgacgcgttttcttaatgaatccatttgcatgcgttcttatgtgaataaactattatatgaatgagtgccaagcaaactgaggctcagacacacctgaccttcctccttcctctctctggctctcacagtgacactctgcgagggacccaacaagttcaagtgtcacagcggcgaatgcatcaccctggacaaagtctgcaacatggctagagactgccgggactggtcagatgaacccatcaaagagtgcggtgagtctcggtgcaggcggcttgcagagtttgtggggagccaggaaagggactgagacatgagtgctgtagggttttgggaactccactctgcccaccctgtgcaaagggctccttttttcattttgagacagtctcgcacggtcgcccaggctggagcgcaatggcgcgatctcggctcactgcaacctctgcctcccaggttcaagtgattctcctgcctcagcctcctgagtagctgggattacaggcgcccaccaccaagcccgggtaattttttgtatgtttagtagagatggggtttcactatgttggccaggctggtgttgaactcctgacctcatgatccgcccacctcggcctcccaaagtgctgggattacaggcgtgacccaccccatgaaaaaaaattaaaaaatgaagcgatgctgggcgcggtggatcacgcctgtaatcccagcactttgggaagctgaggcaggcagatcacgagggcaggagattgagaccatcctggctaatacggtgaaaccccatctctactaaaactacaaaaaattagccgggtgtggtggcaggcacctgtgatcccagctactcaggaggctgaggcaggagaatcgcttgaacccaggaggtggaggttgcagtgagccgggatcacaccattgcactccagcctgggtgacagagtgagactctgtctcaaaaaaaaaaaaaaaaaaaaaagcgaattctgaaatacatgaattcttttccttagatgcctgcttctgtcttgaggtttgttgttgttatttcgaaacagagtcttgctctgtcgctcaggctggagtgcagtggcatgatcttggctcaccacaacctccggctcccaggttcaagcgattcttctgcctcagcctcctgagtagctgggattacagctgaatgccaccttgctgggctaatttttgtatttttagtagagatggggtttcaccatgttggccaggctggcctcgaactcctgacctcgagtgatctgcccgcctcctgaagtgctgggattacaggcgtgagccacctcgtcctggtgagggtttttttttttccccaaccctctgtggtggatactgaaagaccatattaggataactgtacagtatagagaaggcagtggcaagttttctctgtcatataccagagtgggcttgggcatggtggcatactcctgtagtctcagctaatcaggaggctgaggaaggaggatcgcttgggcccaggagttggagactgtagtgagctgtgatcacaccaccacacttcaatctgggcaacagagcaagagaccctatctctaaaaaaaagtaagtatttcggacactgtgggccatacggtctctggtgcagtttctcaacatggctgttgggtgaacacaaccacgcacagaacgcaaaccaatacacgtggctgtgggcccagaaaatgttatttatggacacaaaaattggaatttcatataactgttttgtgtcatgaaaatgatttccctttttatttttatttttcttctcaagtatttaaatatgtaaaagccatttttaggcctggcaggatggttcacagctgtaatcccagcactttgggaggtcgaggcgggaggatcacgaggtcaggagatcgagaccatcctggccaacacagtgaaaccccgtctctactaaaaatacaaaaaattaaccaggcttggtggcgcgcgtctgtagtcccagctgctcaggaggctgaggcaggagaatcgcttgaatgcaggaggcggaggttgtagtgagccgaggttgcaccactgcactccagcctgagcgacagagtgagagtccgcctcaaacaaaaaaatgtttgcccatgctggtcttgaactcctgggctcaagctatctgcctgccttggtctcccaaagttctgggattacaggcatgagctacagcgcccggacttttgttgttttatatctatatatctatatataacttgttttatgtatatatataacttgttttatatatatacataaactgcagtaaaaaacatgtaacataaaatttaccttctcaaaccttattaagtgcacagttctgtgccattagcaaattcacactgttgtacaacatcacaaccaccatctccagaacttttttttttttttttattctttttgagacagagtctcactcgtcgcacgggctggagtgcagtggtgcgatctcggttcactgcaacctccacctaccaggttcaagcaattctcctgcctcagccccctcagtagctgggattacaggtgcccgtcctaccacgcccagctaatttttgtattttcagtagagactgactgggtttcaccatgttggccaggctggtctcgaactcctgacctcaagtgatcctcccacctcagcctcccaaagtgctgggaatacaggcatgagccactgcgcccggccccagaactcttttatcttcccaaactgaagctctgtccccatgaaacactcactctccatcccctccccaactcctggcacccaccattctactttctgtccctatgaatgtgatggctctagggacctcctctgagtggaatcagacagcattttccttttttgactggcttatttcactgagccaagtgcggtggcacacgcctgtaatcccaaaactttgggagaccgaggcgggcgcatcacctgaggtcaggagttcgagaccagcccggccaacatggtgaaaccccatctctagtaaaaatacaaaaaattagcctgtcatggtcgtgggtgcctgtaatcccagctaagtgggaggctgaggcaggagaatcgcttgtacccaggaggcggaggtcgcagtgagccgagatcgtgccattacactccagcctgggcaacaagagtgaaactccgtctctcctaaaaatacaaaaaaattagctgggcatggtggcacatgcctgtagtcccagctacttgggaggctgaggcaggagaatcacttgaacccgggaggtggaggttgtaatgagccaaggttggcggcgaagggatgggtaggggcccgagagtgaccagtctgcatcccctggccctgcgcagggaccaacgaatgcttggacaacaacggcggctgttcccacgtctgcaatgaccttaagatcggctacgagtgcctgtgccccgacggcttccagctggtggcccagcgaagatgcgaaggtgatttccgggtgggactgagccctgggccccctctgcgcttcctgacatggcaaccaaacccctcatgcctcagtttccccatctgttaagtgtgcttgaaagcagttaggagggtttcatgagattccacctgcatggaaaactatcattggctggccagagtttcttgcctctggggattagtaattaagaaatttcaggccgggtgcgtaatccctgtaatcccaacaccttgggacgccgaggcgggcagatcacctgaggtcgggagttccagaccagcctgaccaacatggagaaaccccgtctctactaaaaatacaaaattagccgggcttggtggtgcatgcctataatcccagctactcaggaggctgaggcaggagaatcacttgaacctgggaggtggaggttgtggtgagccaagatcgtgccattgcactccagcctgggcaacaagagtgaaactccatccaaaaaaaaaagaaaagaaaagaaaaaaaagaaaagaaatttcagctgacacagcttcacactcttggttgggttcccgtggtgaatgatgaggtcaggtgatgactggggatgacacctggctgtttccttgattacatctcccgagaggctgggctgtctcctggctgccttcgaaggtgtgggttttggcctgggccccatcgctccgtctctagccattggggaagagcctccccaccaagcctctttctctctcttccagatatcgatgagtgtcaggatcccgacacctgcagccagctctgcgtgaacctggagggtggctacaagtgccagtgtgaggaaggcttccagctggacccccacacgaaggcctgcaaggctgtgggtgagcacgggaaggcggcgggtgggggcggcctcaccccttgcaggcagcagtggtgggggagtttcatcctctgaactttgcacagactcatatcccctgaccgggaggctgtttgctcctgagggctctggcaggggagtctgccgccctgttaggacttgggcttgccagggggatgcctgcatatgtcctagtttttgggaatatccagttaacggaaccctcagccctactggtggaacaggaaccggctttcctttcagggacaacctggggagtgacttcaaggggttaaagaaaaaaaattagctgggcatggtgccacacacctgtggtcccagctactcagaaggctgaggcgggaggattgcttgagggcaggaggattggttgatcctcccacctcagcctccggagtagctgggacctcaggtgcatgccactatgcctggctaattttcttttttctttttttttttttttcgagacggagtctcgctctgttgcccaggctggagtgcagtggcaggatctcggctcactgcaagctccgcctcccgggttcacgccattctcctgcctcagcctccccagtagctgggactacaggagcccgccactgcaccaggccaatttttttgtatttttagtagagacggggtttcactgtgttagccaggatggtctcgatctcctgacttcgtgatccgcccacctcggccttccaaagtgctcggattacaggcgtgagccactgcgcccagccgctaattttcatatttttagtaaaaacagggtttcaccatgttggccaggctagtcttgaactcctgaacccaagtgatcctcctgccttggcctcccaaagtgctgggattacagacaccacacctggctattattattttttagagacagggtgctgctctatcttccagcctgtagtgcagtgcagcctccatcatagctcgctgcagccttgacctcctgggttcacgtgatcgtcccgcctaagcctctggaggagctgggagtactggcatgtgccaccatgcctggttaatttttttttttttttttttgagacagagtctcattctgtcacccaggctggagtgcggtggtgcgatcttggcttactgaaacctccacctcccaggttccagcaattctcctgcctcacccttctgagtagctgggattacaggttccggctaccaaacctggctagtttttgtatgtttagtagagacagggtttcaccatgttggtgaggctggtctcgattctcccgcctcagcctcccaaagtgctgggattacaggcttgagccaccgtgcctggcttttttttttttttttttttttgtggcaataaggtctcattgtcttgcccaggctagccttatgctcctagcctcaagtgatcctcctccctcagcctcccaaagtgctgggattacaggtgggcgccactgtgcctgttcccgttgggaggtcttttccaccctctttttctgggtgcctcctctggctcagccgcaccctgcaggatgacacaaggggatggggaggcactcttggttccatcgacgggtcccctctgaccccctgacctcgctccccggacccccaggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcaggtgagcgtcgcccctgcctgcagccttggcccgcaggtgagatgagggctcctggcgctgatgcccttctctcctcctgcctcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatgggtgcgtatccacgacgctgagggctgcagagggaatggagggagcaggaaggagcttcaggaactggttagtgggctgggcatggtggctcaaagcacctgtaatcccagcactttgggaggccaaggtgggtggatcatcaagaccagcctgaccaacatggtgaaacctcgtctctactaaaaatacaaaaattagccgggtgtggtggtgggcacctgtaatcccagctgctcgggaggctgaggcaggagaatcacttgaacctgggagatggaggttgcagtgagccaagacagccccactgcactccagcctgggtgacagagtgagactccgtctcaaaaaaaaaaaaaaaaactaaacaaaaaactggttagtggctagacaacaggatggtatcttccaagcccatggctgactcagcagctcctgggtcaagacactgtgacctgtgtcccctggcaggaagcatcgcccctgccacctgcccggtgtactctgtacctgtcaggtgacatctgctacctaagcacgtgagaggtggcatttcacagtttcagtgtggtgctgacaacccgggacgcacactgtccttgcagctacaatcaggaggtgaatgttgggtttccagcagagaacactggagaaggcacacttggtgtctggaagggaaaagcagggaagagagcatcatcagatgcctgcgggtgaaggtgggcccgctatggccagcgtccctttttatttttatttatttatttatttgagatggaatctcgctctgtcgcccagactgtagtgcagtggtgcgatcacggctcactgcaagctccgcctcacaggttcacgccattctcctgcctcagcctcccgagtagctgggactacaggcacccgccaccacgcccggttaattttttgcatttttattagagacggggtttcaccgcgttagccaggatggtctaaatctcctgaccctgtgatccacccgcctcggcctccctaagtgcttggattacaagcgtgagccaccacgcccggccccctttttattttttattttttgagacggagtctcgctctgtcgcccaggctagattgcagtggcgtgatctcggctcactgcagcctccgcctcccaggttcaagtgattctcctgcctcaacctcccaactaattaggattacaagcatgtaccaccatgcctgactaattttttgtatttttagtagagactgggtttcaccatgttggctaggctggtctcgaacccttagcctcaagtaatctgcctgcctcagcctcccaaacagcggggattacaggcatgagccactgtgcccaacccaaccctggatctcttttaaacaagacaatgctcgctgttgccacagaacaatgggtggggtacatgtggcccagtgtgtttggccacataactgccaggccagagggaaagagactctcagactgtctccactcagatacaaatgtgtgtgttgtgtgcgtgtgttctggtctcatatttgtttgttttgagacagggtgtcgctctgtcactgagtctggagtgcagtggcgcaatcagagttcactgcagcctcaaactcttgggctcagttgattctcccacttcagcctcccaagtagctggaactacaggtgaacaccactgtgcccagctaatttattttatttttagtagagatgaggtctcactatgttgcccaggctggtcttgacctcctagcctcaagcaatcctcctgccttggtctcccaaagtgctgggattacacgtgcgagccattgcgcatggcttgtgttcttgtgtttcttcctttttctttcgagatggcgtctcagtctgccacccaggctggagtgcagtggtgtgatcatagctcactgtagcctcaacttcctgggctcaagcaatcctcttgatttcagcctcccgggcctggccagcatggtgaaaccccgtctctactaaaaatacaaaaatgtagccaggcgtggtggtgggcgcctgtaatcccagctacaccagaggctgaggcaggagaatcgcttgagcctggaaggtggaggttgcagcaagccaagatcgtgccactgcactccagcctgggcaacagagacagactctgtctcaaaaaaaaaaaaaaaaaacccaaacaagccacatttggagtttggggttcccagcaggactatttcccaagcctgagcctggctgtttcttccagaattcgttgcacgcattggctgggatcctcccccgccctccagcctcacagctattctctgtcctcccaccagcttcatgtactggactgactggggaactcccgccaagatcaagaaagggggcctgaatggtgtggacatctactcgctggtgactgaaaacattcagtggcccaatggcatcaccctaggtatgttcgcaggacagccgtcccagccagggccgggcacaggctggaggacagacgggggttgccaggtggctctgggacaagcccaagctgctccctgaaggtttccctctttcttttctttgttttttctttttttgagatgaggtcttggtctgtcacccaggctggagtgcactggcgcaatcgtagctcactgcagcctccacctcccaggctcaagtgatcctcctgcctcaccctcctgagtagctgagattacagacacgtgccaccacggcagactaattttattttatttttgggaagagacaaagtcttgttatgttggcctggctggtctcaaactcagggtgcaagcgatcctcccgcctcagccttccaaactgctgggattacaggcgtgggccaccgtacccagcctccttgaagtttttctgacctgcaactcccctacctgcccattggagagggcgtcacaggggaggggttcaggctcacatgtggttggagctgcctctccaggtgcttttctgctaggtccctggcagggggtcttcctgcccggagcagcgtggccaggccctcaggaccctctgggactggcatcagcacgtgacctctccttatccacttgtgtgtctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggtgtggcttacgtacgagatgcaagcacttaggtggcggatagacacagactatagatcactcaagccaagatgaacgcagaaaactggttgtgactaggaggaggtcttagacctgagttatttctattttcttctttctttttttttttttttttgagacagagttttgctctcgtttcccaggctggagggcaatggcatgatctcggctcaccgcaacctccacctcccaggttcaagtgattctcctgtctcaggctccccagtagctgggattacaggcatgcaccaccaccatgcccggctaattttgtatttttagtagagacggagtttctccatgttggtcaggctggtctcgaactcccgacctcaggtgatctgcctgcctcggcctcccaaagtgctgggattacagacttgagccaccgcgcccagctatttctgttttctttctttcttcttcttctttttttttttctaagagacaggatctcactctgtccccaggcaggagtgcagtgctgtgatcatagctcactgcagccttaacctcctgggctcaagtgatcttcccacctcagcctcccaagtagctggaactacaggtgcacaccaccatgcccagctcatttttgtattttttttttttttgagacagtctcgttctgtcaccccggctggagtgcagtggtacaatcttggctcactgcaacctctgcctcccaggttcaagcgattctcctgcctcagcctcctgagtagttgagattacaggcatgtgtgccatcatacctggctgatttttgtatttttttttagagatggggtctcagtatgttgaccaggcttgtcttaaactcccggcctcaagtgatcctcccacttcagtctcccaaagtgctgggattacaggcatgagccactgcggccggtttgttttcttttttttttcgttttttggagacggaatttcacctttgttgcccaggatggagtgcaatggcacgatatcgcctcaccacaacctctgcctcctgggttcaaaccattttcctgcctcagccttcttagtagctgggattacaagcatgtgccaccacgcccggctgattttgtatttttagtagagatggggtttctccatgttggccaggctggtctcgaactcctgacctcaggtcattcgcccacctctgcctcccaaagtgctgggattacaggcgtgagccaccgtgcccggtggtttgtattctttttactgagagtcgtgaaaggcagtgatcctctgtcacatgtgatcttggctctcaggggacatttggcaatttctagagattttttggttgtcacaagtcaatggggaagactgttggcatttagtgggtagaggctggtgacgctgctgaacacccagaacagggaagtagcaggccctagatagagccatcgtggggaaaccctgctctaaggaaatggcgctattttataaccccacgttcctggcatgattaccaacagccaaaagtggagtccccccaagtgtgttcgtccatttgcattgcagtaaaggaatagctgaggccgggtaatttataaagaaaagagatttaaactgggtatggcagtttatgcctataatcccagaactttgggaggctgaggcaggaggatcgcttgagtccaggagtgtgagaccgagaccagcctggccaacatgacgaaactctgtctctacaaaaaatacaaaaagtaggccaggcacggtggttcacgcctgtaatcccagcactttgggaggccgaggcgggcggatcacgaggtcaggagatcgagaccatcctggctaacacggtgaaaccccgtctctactaaaaatacaaaaacaaaattagccgggtgtggtggcaggcgcctgtagtcccagctactcgggaggctgaggcgggagaatggcgtgaacccgggaggcggagcttgcagtgagccaagatcgcgccactgcactccagcctgggtgaccgagttgagactccgtctcaaaaaaaaaaaaaaaaaaaaaaatacaaaaagtagccaggtgtggtggcaggcacctgtaatcctgggttctcgagaccgaggcatgagaattgcctgaccccaggaggtggaggctgcagtgagccaagatcatgccactgcactccagcctgggcgacagagtgggactctgtctcaaaaaacaacaaaaaaaaagttctggaaatggatggtggtgatggtgatacttccacaacagcgtgaatctgcttaaggccaccgaactgtgcactcacaaatagtcgagatggtacattttatgttatgtgtatttcaccacaattaaaaactagttgtgggccaggtgtggtggttcatgcctgtaatcccagcactttgggaggtcagagggaggtggatcatgaggtcagcagttcgagaccagccaggccaacatggtgaaaccccatctctactaaaaatacaaaaattagccaggcgtggtggcacatgcctgtagtcccagctacttgagaggctgaagcaggagaatcgcttgaacctgggaggctaagattgcagtgagccgagatcgtgccactgcactccagcctggacgacagagtgagacttcgtctcaaaaaaaaaaccaaaaaaaaaattagctgtgggtcaggcactgtggctcacgcctgtaatcccagcactttgggagaccgaggtaggtggatggcctgaggtcaggagttcgaatccagcctggccaacatggtgaaagcccgtctctactaaaaatacaaaaaattagtcaggtatgttggcacacctgtaatcccagctactcgggaggctgaagcaagagaatcgtttgaacccaggaggtggacgttgcagtgagccgagattgggccactgtactccagcctgggcaacaaaagtgaaactctgtctgaaacaaacaaacaaacaaacaaacagacaaacaaaaaaactagttgtggagagagggtggcctgtgtctcatcccagtgtttaacgggatttgtcatcttccttgctgcctgtttaggacaaagtattttggacagatatcatcaacgaagccattttcagtgccaaccgcctcacaggttccgatgtcaacttgttggctgaaaacctactgtccccagaggatatggttctcttccacaacctcacccagccaagaggtaagggtgggtcagccccacccccccaaccttgaaacctccttgtggaaactctggaatgttctggaaatttctggaatcttctggtatagctgatgatctcgttcctgccctgactccgcttcttctgccccaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacaggtgtggcacacgccttgtttctgcgtcctgtgtcctccaactgccccctcctgagcctctctctgctcatctgtcaaatgggtacctcaaggtcgttgtaaggactcatgagtcgggataaccatacttttcttggatggacacatcagcaccgggcttgacatttacccagttcccctttgatgcctggtttcctctttcccggccccctgaagaggtgatctgatttctgacaggagccctgagggaggaaatggtcccctttgttgacttttctttttctttatttttttcttttgagatttgctgtcacccagcctggaatgcagtggtgccatcttggctcactgctacctctcccactgggttcaagcaattctcctgcctcagcctcccaagtagctgggattacaagcatgcgccaccatgcctggctaagttttgtatttttagtacagacagggtttctccatggtggccaggctggtcttgaactcctgacctcaggtgatcctcccacctctgcctcccgaagtgctacgattacaggcatgagccaccgcgcccatccccctttgttgacttttctcatcctctgagaaagtctcagttgaggccagcacctccctcaagtgaattgaatctcccttttgaacaacaacaaataacaatatgacccagacgtggtggctcacacctgtggtcccagctactcgggaggctgaggtgtgaggattgcttgagcccaggaggtcaaggctacagagagctataatcacaccacttcactccagcctgggggacaaagtgaaaccctgtctgaaaaaaacaaaaaaagaaaaaggaaaaagaaacaatacgatcacaaagtagatattcatagtgtttattttcagtactcttttttttttttttttttttttttgagacggagtcttgctctgttgcccaggctggagtgcagtggcacgatcttggctcactgcagcctctgcctcccaggttcaagcgcttggctcactgcaacctccgcctcctgggttcaagcgcttcttctgcctcagcctccccagtagctgggactataggcacgtcccactacgcccagctaattttttgtattttttagtagagatggggtttcactatgttagccaggatggtctcgatctcctgacctcgtgatctgcctgccttgggctcccaaagtgttgggattatgggcatgagccactgcacctggccttttttttttttttttttgagatggagtttcgctcttgttgcccaggctggagtgcaatggtgtgatctcggctcactgcaacctctgcctcctgggttcaagcaattctcctgcctcagcctcccgagtagctgggattacaggcacctgccaccacgcctggctaatttttgtacttttagtagagacggggtttctccatgttggtcaggctggtctcaaactcctgacctcaggtgatccacccacctcggcctcccaaagttctgggattacagacatgagccaccgcgcctggccgtgtctggccttttttagttatttcttttttttttttttttttttttgagacagagtcttactccgtcgcccaggctggagtgcagcggtgcgatgtctgcgcactgcaagctccgccccctgggttcatgccattctcctgcctcagccttctgagtagctgggactgcaggcgcctgccactacgcccggctacttttttgtatatttagtagagatggagtttcactgtgttagccaggatggtctcgatctcctgactttgtgatccgcccgcctcggcctcccaaagtgctgggattacaggcgtgagccaccatgccaggcttttttttttttttttttttttgagacggagtcttgctctgtcgcccaggctggagtgcagtgccatgatctcagctcactgcaagctccacttcccaggctcacgccattctccagcctcagcctcccaagtagctgagactacaggggcccgccaccacactcggctaatttttttgtatttttagtagagacggggtttcaccatgttagccaggctggtcttgaactcctaacctcaggcgattcacctgcctcggcctcccaaagtgctgggattaaaggtatgagccacctcgcctggtgtgagccacctcgcccagcctgagccacctcacccagcctaagccactgtgcctggcctgattttggactttttaaaaattttattaataattatttttgggtttcttttttttgagacagggtcttactctgtcatccaggccatcctgtctgtctgtcatcccagtgatgggatcataccttgctgcagcctctacctcctgggctcaagcgatcctcccccctcagcctcctgagtagctgggagtacaggtgtgcaccaccacacctggctaatttttttttttttttttgtatatagagatggtattttgccatgttgaccaggctagtcttaaactcctggactcactcaagagatcctcctgccttggcctcccaaggtcatttgagactttcgtcattaggcgcacacctatgagaagggcctgcaggcacgtggcactcagaagacgtttatttattctttcagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaaggtaaagactgggccctccctaggcccctcttcacccagagacgggtcccttcagtggccacgaacattttggtcacgagatggagtccaggtgtcgtcctcactcccttgctgaccttctctcacttgggccgtgtgtctctgggccctcagtttccctatctgtaaagtgggtctaataacagttcttgccctctttgcaaggattaaatgggccaaatcatatgaggggccaggtccttcaggctcctggttcccaaagtcagccacgcaccgtgtgggtcccaaaattttatcaaggcacattcgttgcctcagcttcaggcatctgcccaaaaaggccaggactaaggcaaggagagggagggattcctcagtactcagcttttcacagaggctccaaaaggctaaggaatccagtaacgttttaacacaattttacaatttttttttttgagacggagttttgctcttgttgcccaggctggagtgcagtggcacgatctcggctcactgcaacctctggctcccgggttcaagcgattctcctgcctcagtctcccgagtagctgggattacaggcatgcgccaccacgctcggctaattttgtatttttagtacagaaggggcttctctgttggtcaggctggtcgtgaactctcaacctcaggtgagccacccgcctgagcctcccaaagtgctgggattacaggtgtgagccaccacgcctggccttttttttgagacagagtctcgctctcgcccatgctgtactgcagtgacgcagtctgggctcactgtaacctccgcttcccaggttcaagtgattcttctgccgcagcctcccatgtagagtagctgggattacaggcacccgccaccatgcctggctaattcttgcatttttagtagagatggggtttcacagtgttggccaggctggtctcaaacttctgacctcaagtcatctgcctgccttggccctgccaaagtgctgggattatagatgtgagccaccgcgcctggcctacagtttattctttggtggctcacacctgtaatctcagcactttgggaggccaaggtgggagaatggcttgagcccaggagttcaagtccagcctgggcaacatagcaagaccctatctctactacaaaataaataataaataaactaattttttttcttttaaaacccaactattcaacatggcaatgcaatatattaaaaaaattttttttttctttgaaacggagtctctcactgtcacccgggctggagtgcagtgtcgccatcttggctcactgcaacctccgcctcccaggtccaagtgattctcctgcttcagcctcccgagtagctgggattacaggcacccaccaccatacccagctaatatttttgtatttttagtagagatggggtttcactatgttgggcaggctggtctggaactcctgacctcgtgatctgcccgaggatcggcggcctcccaaagtgctggggattgcaggcatgagccaccgtgcccagccaaaacttttttatttttatttttttgggacacggtctcactgtgtaccccagactggagtgatagagtgctgtcatggctcactgcagcctcaacctccctgggctcaggtgatcttcctgcttcagtctcccaggtagctgggactacaggcatgagccaccacacccagctaatttttgaatttttttgtagagacagggtttcaccttgtggcccagacttgtctctaactccagggctcaagcgatctgcccaccttggcctcccaaagtgctgagattaatgcaatttaaaaaattttttggccaggcctggtggctcatgcctgtattcacaacaccttgggaggcaaaggtgggcagatcacttgaggtcaggagttcgagactagcctggccaacatggtgaaaccccctgtctactaaaaaaatacaaaaattacctgggcacagtggtgggtgcctgtaatcccagctacttgggatgctgagggtggagaattgcttgaacctgggaggcagaagttgcagtaagccaagatcatgccactggactccagcctcagtgacagagcaaaactctgtctccaaaaaaattgttttttttttttttttttcaaatcatcacactacagccaaggcctggccacttacttttgtaaataaagttttattggagccagtggaccagtgaggccgaatcttgcaggtgtaagatcacagtctatccttgaaaattttgatattttgttcattgggtggtttttcattaatttaaattttaaaaaataacatattaaaggctggtgtggaggtgcacgcctgcagtcctagctactcccagaggctgaggcgggagacttgcttgagcccaagagttgaagtccagcctgggcaacatagcgagacccccatctctaaaaataaaaataatgcattagaatattattggattcctgggcagggcacagtggctcacacctgtaatcccagcactttgggaggctgaggtgggtggatcacctgaggtcaggagtttgagaccagcctggccaacatggtgaaaccccgtctctactaaaaatacaaaaattagccaggcgtggtggcaggtgcctgtaatcccagctactcgggaggctgaagcacgagaatcgcttgaatccaggaggcggaggttgcagtgagctgagattgcgccattgcactccagcctggaggacaagagtgaaactccattcccctctgcaaagaaaaggaatattatcagattcctaagctttttggctccccctttagtttgggggctggggtggtgagtgtctgacctggcctcactgtcctccctggatgtgatgagacccaggtgtgggtcaggatgtcattcgtttgtccaccagagggcgcccaaactgctttgagctgctgggaaatggtgctcctagacttttagcaaacaaacaaaaaaaaatggcacatcggcaaatttcagaccattctttttttttttttttttggttccagagtagctgaaatctttgttcagttacaagcaggataaaatggaaactgcctgggagaggctgagaaaccttcttgcttgggggaggtggggcactgctagaattaatcgcttcacagaccagcccatccaggactcctcaaatttggcaaaaaagccattcattcattcattcatttatgtagagacgagggggatctggctatattgcctagattggtctcaaattcctggcctcaagtgatcctcctgccttggtctactaatgtgctgcgattacaggcatgagccaccgtgcctagctctagtggacttgaaatgttgccttgcccagggcccttatgttgaatggcccaggtccacttgtatggttctgtaccaaggttaaccccatcccataatgcctgggacagttgatgcaggacaatcagcttctgtgccattcaacctcaggactgagcatgctgggcattgtggggtccgaaggtggctcccctgtccccttcaaaataccctctttttcttttcttcttttttttttttttttttttttgagacgaagtcttgctctgttgccccagctagagtgcagtggtgcgatctcagctccccgcaacctctgcttcccgggttcaggcgattctcctgcctcagcctcctgagtagctgggattacaggtgcccaccgccacagctggctaatttttgtatttttagtagagacagggtttcaccgtgttggccaggctggtcttgaactcctgacctcaggcaacctgcccacctcagcctcccaaagtgctgggattacaggtttgagccactgggcctggccttttttttttttttttgagagggagtctcactctgttgcccaggctggagtgcaatggcgcgatcttgactcactgcaactccatttcccgggttcaagtgattctcctccctcagcctcccaagtagctgggattacaggtgcatgccaccacggccagctaattttgtatttttagtagagacagggtttcactatgttgatcatgctggtctcaaactcctgaccttaggtgatctgcccgccttagcctcccaaagtgttgggattacaggtgtgagccaccgcgcccagaccaaaatatgctcattttaataaaatgcacaagtaggttgacaagaatttcacctgcaaccttgtcaaccacctagaataaaagcctctgcagccctcccctaaagactcatcaatgtgaggctcaagaaccttcttaggctgggctcggtggctcatttctgtaatccctgcactttggaaggctgaggcaggaggatctcttgaggccaggagttcaagacaagcctgggcaacatagccagacctctgtttctatcccccacaaaaagaaccttcttaaaccggaattgagtcctacaacctcgataactcacaaataagcccgtgtggcctctcacagacttgggaagttctccaagtgtccagggagatgtgccaggcgctttcctgccgtgaccaccgtcctctgcctgctccatttcttggtggccttcctttagacctgggcctcactcttgcttctctcctgcagctctgggcgacgttgctggcagaggaaatgagaagaagcccagtagcgtgagggctctgtccattgtcctccccatcggtaagcgcgggccggtcccccagcgtcccccaggtcacagcctcccgctatgtgacctcgtgcctggctggttgggcctgttcactttttctcctggacagggaacagccccactggtgtcctttatcacccccacggcctctcctggcttggggctgacagtgacaagatcagacagctaaggggtcagatggaggatgtggagctgggtcccgtgctgtggaatagcctcaccgagatttgagtgccttctggggaactggttcccttgcagggggctgtgtggagaggcgcgctctccctgcctcacccatgctcatcctaactcggttaccatcacatctcttttttctttttttcttaaattttaagaaaaaagaaatttaatttttttgagagacagagtcttgctctgtcacccaggctggagtgcagtggcaccatcatgcctcgctgcagcctcaatgtctgggctcaagcgatcctcccacctcagcctcctgagtagctggtgcaagccactataccccacttcctatttcttaaaaagtcacagccctgtgtgtggctaatcctggacagaaatctagaagaagtcagctacttctggggcgtggctcacccagtgggcttcaggttagatatttcttatacttatgaggctgggtgtggtggcttatgcctgtaatcccagcactttgggaggctgaagtgggtggattgcttgggctcaggagttcgagaccaacctgggcaacatggcgaaaccctgtttctagaaaaggtacaaaaattagctgggcaggtggcacgtgcctgtggtaccagctacttgagggcctgaggcaggaggatcgcttgaacctgggaggtcgaggttgcagtgaactgagatcatgtcactgcactccagcctggtgacagagcaagaccccgtctcaaaaaaaaaaaaagaaagaaaaaaattcttatgcatagatttgcctcttttctgtttgtttgttttgagatggagtctcgctctgtcgcccaggctggagtacagtggctcaacctcggctcactgcaacctctgcctcccgggttcaagcaattctcctgcctcagcctcctgagtagctgggactacaggcgcccgccaccatgcccagctaatttttgtatttttagtagagactgactgggtttcatcatgttggccaggctggtctcgaactcttgacctcatgatccgcccgcctcagcctcccaaaatgctgggattacaggcgtgagccaccaggcccaggccgcaaggcgatctctaaacaaacataaaagaccaggagtcaaggttatggtacgatgcccgtgttttcactccagccacggagctgggtctctggtctcgggggcagctgtgtgacagagcgtgcctctccctacagtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcggtgagtgaccctctctagaaagccagagcccatggcggccccctcccagctggaggcatatgatcctcaagggaccaggccgaggcttccccagccctccagatcgaggacagcattaggtgaatgcttctgtgcgctcattcagaatgtcagcggacaatggccttggtggtgtagaggaatgttggataagcaaatagagagctccatcagatggtgacagggcaaagaaagtcaaaaggagttcagaggccgggcgcggtggctcatgcctgtaatcccaggactttgggaggccgaggctggcggatcacctgaagtcaggagtttgagaccagcttggccatcatgacaaaaccccgtctctattaaaaatacaaaaaattagccaggcgtgggagtgggcgcctgtaatcccagctactcgggaggccgaggtagaaaaatcgcttgaacctaggaggcagaggttgcagtgagccgagatcgcgccactgcattccagcccgggaggcaagagcaaaactccatctcaaaaaaaaaaaaaaaaggagttcagaggcccggcatggtggttcacacatgtgatcccagaacttggggaggttgaggcaggagaatcacctgagctcagagttcaagaccagcctgggcagcacagcaagaccccatctctgcaaaaaataaaaatttagcccagtgtggtgatgagcgcctagttccagctactagggaggctaaggcaggaggattgcttgaggctaaggtaggagattgagactgcagtgacttgtgattgcgtcactgcgctccagcctgggtgacagagcaagcccttgtctcttaaaaaaaaaaaaaaattcaaagaagggtttccagagggccaggagggaggaagggagaggaggtgttttatttttttgcttttattttttattttgagacagagtctctctctgtcacccaggttggagtgcagtgctgtgatcttggctcactgcaacttctgcctcctgggttcaagcaattcttatgcctcagcctcagcctcctgagtagctgggattacaacactatgcccgggtaatttttgtatttttagtagagacgaggtttcgccatgttgcccagactggtctcgaactcctgacctcaagtgatccacccgccttggcctccccacgtgctgggattgcaggcgtgagccactgcgcccgccttgatctttacacaaggggtttagggtaggtagccttctctgaaccaggagaacagcctgtgcgaaggccctgaggctggaccgtgcctgttgggtttgaggccgttgtagctggagcaaacagagagaggggtaaaaaggcaggaggctaccaggcaggttgtgcagagccttgtgggccactggggaggactttggcttttgccctgagagcggtgggaagtgactgaatccggtactcaccgtctccctctggcggctcctgggggaacatgcttggggatcaggctgggggaggctgccaggcccaggaggtgagaagtaggtggcctccagccgtgtttcctgaatgctggactgatagtttccgctgtttaccatttgttggcagagacagatggtcagtctggaggatgacgtggcgtgaacatctgcctggagtcccgtccctgcccagaacccttcctgagacctcgccggccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaaggggaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccgaatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcactggtcacccttaatatttattaagtgcctgagacacccggttaccttggccgtgaggacacgtggcctgcacccaggtgtggctgtcaggacaccagcctggtgcccatcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggctcacgcctgtaatcccagcactttgggaggccgaggcgggtggatcatgaggtcaggagatcgagaccatcctggctaacacgtgaaaccccgtctctactaaaaatacaaaaaattagccgggcgtggtggcgggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaaaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtacggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaatgaaccaatttgtctaaactcgatgcacgttcttctgttcgcgcgcttctttttgtttttttttttttcctgagatggagcctggctctgtcacccctggctggagtgcagtggcatgatctcggcttactgcaagctccgcctcccaggttcaagcaattctcctgcctcagcctccctagtagctaggattacaggtgagtgccaccacgcctggccaattttttttttttttttttttttgagacagagtctcgctctgtcacccaggctggagtgcagtggtgtgatctcggctcactgcaagctctgcctcccaggttaatgccattctcctgtctcagcctcctgagtagctggggccacaggcgcctgccaccacgcccggctaatttttttttgtacttcttttagtacagacggggtttcaccatgttagccaggatggtctcgatctcctgaccttgtgatccacctgcttcggcctcccaaagtgctgagattacaggcgtgagccaccgcgggtggccaacgctaatttttttgtttttttagatggagtcttgctctgtcgcccaggctggagtgcagtggcgtgatctctgcctactgcaagctccgcctcccgggttcatgccattctcctgcctcagcctcctgagtaactgggactacaggcacccgccaccacgcccggctaattttttgtatttttagtagagacagggtttcaccgtgttagccaggatggtcttgatctcctgaccttgtgatccacccgtctcggcctcccaaagtgctgggattagaggtgtgagccaccacacctggcctagcctggctaatttttgtatttttggtagagacggggtttcaccatgttggtcaggctggtcttgaacttctgacctcaggtaatctgcctgcctcagtctcccaaagtgctgggattacaggtgtgagccaccgcgcctggcctcacttccttctgtcatctgtttgtggattggactccccaggagaaggacccagaaggggaagactcccagaactccgggcaagatgcaatctccgtgggctgccacagtgcctggcaggtgctgtgatggctgagctggtgattgtgttctctgctgtcgcttctctgagttggagattttgtcaagtcccctgctcatccattcatacactcgacaaatatctgttgagtgctaagtgcgaaccatgctctgccgtaggcttgtgggacactacaggggatataagaaatgaaagccgggtgtggtggctcacacctgtaatcctagcagtttgggaggccgaggcgggcagatcatgaggtcaggagatcgagaccatcctagctaacacagtgaaaccccatctctactaaaaatacaaaaaattagccaggcgtggtggtgggcgcctgtagtcccagctgcttgggaggctgaggcaggagaatagcgtgaacctgggagttggagcttgcagtgagccgagatcgcaccactgcactctagcctgggcaacagagcaagactccatctacaaaaaaaaaaaaaagaaatgaagtcttgatacggtggctcatgcctgtaatcccagcactttgggaggccaaggcaggcggatcacgagctcaggagatcgagaccatcctggccaacgtggcgaaacccagtctctactaaagatacaaaaaattagccaggcatggtggcggg

SEQ ID NO: 2 - RNA, 1200 nt, Homo sapiens
gggaccaacgaaugcuuggacaacaacggcggcuguucccacgucugcaaugaccuuaagaucggcuacgagugccugugccccgacggcuuccagcugguggcccagcgaagaugcgaagauaucgaugagugucaggaucccgacaccugcagccagcucugcgugaaccuggaggguggcuacaagugccagugugaggaaggcuuccagcuggacccccacacgaagrccugcaaggcugugggcuccaucgccuaccucuucuucaccaaccggcacgaggucaggaagaugacgcuggaccggagcgaguacaccagccucauccccaaccugaggaacguggucgcucuggacacggagguggccagcaauagaaucuacuggucugaccugucccagagaaugaucugcagcacccagcuugacagagcccacggcgucucuuccuaugacaccgucaucagcagrgacauccaggcccccgacgggcuggcuguggacuggauccacagcaacaucuacuggaccgacucuguccugggcacugucucuguugcggauaccaagggcgugaagaggaaaacguuauucagggagaacggcuccaagccaagggccaucgugguggauccuguucauggcuucauguacuggacugacuggggaacuccygccaagaucaagaaagggggccugaaugguguggacaucuacucgcuggugacugaaaacauucaguggcccaauggcaucacccuagaucuccucaguggccgccuyuacuggguugacuccaaacuucacuccaucucaagcaucgaugucaaygggggcaaccggaagaccaucuuggaggaugaaaagaggcuggcccaccccuucuccuuggccgucuuugaggacaaaguauuuuggacagauaucaucaacgaagccauuuucagugccaaccgccucacagguuccgaugucaacuuguuggcugaaaaccuacuguccccagaggauaugguycucuuccacaaccucacccagccaagaggagugaacuggugugagaggaccacccugagcaauggcggcugccaguaucugugccucccugccccgcagaucaacccccacucgcccaaguuuaccugcgccugcccggacggcaugcugcuggccagggacaugaggagcugccucaca

SEQ ID NO: 3 - DNA, 49020 bp, Homo sapiens
tccgcctcctgggttcatgccattctcctgcctctgcctcatgagtaactgagactacaggcgcccaccaccacgcccggctaatttttttgtatttttttagtagagatggggtttcaccttgttagccaggatggtctcgatctcctgacctcgtgatccacccgtctcggcctcccaaaatgctggcattacaggcgtgagccaccgcacccagccttaaatttttttttaagggaaatcaaacccagtgatattgggccagtacagtggctcacacctgtaattccaccactttgggaggctgaggcaggtgaatcacctgaggtcaggagttcgagaccagcccggcaaacatggcgaaaccccgtctctactaaaaataagaaaattagccgggcgtagtggcatgcacctgtaatctcagctactcgggaagctgaggcatgagaatcgcttgaacctgggagcaggatgttgcagtgaaccgatatcacaccactgcactccagcctgggtgacagagcaagactctgtctcaaaaaaaaaaagaaaaaaaaatccagtgatacttactttttaaatttttatttacttattttttgctttaagttgaatctttaaacttatctttatttttgagacacagtctcactctgtcgcccaggctggagtgcagtggtacaaccacagctcagtgcagcgttgacctcctgggctcaagccatcctcccgcctcagcctcccgagtagctgggactacaggcgcacacaaccatgtccagcttatttttgtattttttgtagagacagggtcccactgtgttgccctggcttgttctgaactcctaggctcaagtgatccccccgcctcaccctcccaaagtgctgggattacaggcatgagccaccacatccagacttcacttttttgtttaatgtcgcaaatggcataaggaatgggattcaatggggacacatttataaacgttgcagcagctcctagaacttgcctatccttgtaaacttctctaggtgattgctaattacttctttttttttttttttttttgagacggagtctcactctgtcgcccaggctggagtacagtggcgcaatctcgtctcactgcaaactccacctcccgggttcacgccattctcctgcctcagcctcccgagtagctgggactacaggcacccgccaccacgcccggctaattttttgtatttttttttagtagaggtggggtttcactgtgttatccaggatggtcttgatctcctgacctcgtgatccacctgcctcagcctcccaaagtgctgggattacaggcgtgagccaccatgcccagcccgctaattatttcaatttgaccttgacactgagcctgccaagtaggttcaagcattttgatggcccctttacaggttgggaaagctaatttatctgtccaaggccgaattctgaaactgagtcttaactgccaaaaattcttatcatcaatttcttcttctgggttgggcacagtggctcatgcctgtaaagccagcaatttgagaggcatcatgatgcaagaggaagaggattgagtgaagctaggagtttgggaccagcctgggcaacatagtgagaccccatctataaaaaaaaattaaaaattagttgggcatggtggtgcactcctgtggtcctagctattcaggaggctgaggtgggaggattccttgagcccagggttgacgctgcagagagctgtgatcacgccactgcagtccagcctgagtgacagctggaaataatgataaataaataataaataattatttaaaaaattataataaaaataattaaaaaattattttccctgattaatctttttttttgtccttctgagagttcaatttgtcccttttctgcctggtctcctaggtttccctaaaatcctgctgagaggttagcactgcctgccaaagtcagtttgcaaaatcccagagaaatccagcttattcctgggggaaccgccaagactgcccagccctgtgtggggttcaggcaagtttctcacatgtgcctttttggcaagaggcctctggcaaccccatgagtccccaaagagactcaattctaaaagttggtctccaccagctctctgtggcttaggggttcaagttcaactgtgaaagccctgttttgttttgattttgctttgagggagaggaaaccgcccttctgtttgttcaactccttctcctaaggggagaaatcaatatttacgtccagactccaggtatccgtacaattgatttttcagatgtttatactcagccaaaggcgggatcccacaaaacaaaaaatatttttttggctgtacttttgtgaagattttatttaaattcctgattgatcagtgtctattaggtgatttggaataacaatgtaaaaacaatatacaacgaaaggaagctaaaaatctatacacaattcctagaaaggaaaaggcaaatatagaaagtggcggaagttcccaacatttttagtgttttccttttgaggcagagaggacaatggcattaggctattggaggatcttgaaaggctgttgttatccttctgtggacaacaacagcaaaatgttaacagttaaacatcgagaaatttcaggaggatctttcagaagatgcgtttccaattttgagggggcgtcagctcttcaccggagacccaaatacaacaaatcaagtcgcctgccctggcgacactttcgaaggactggagtgggaatcagagcttcacgggttaaaaagccgatgtcacatcggccgttcgaaactcctcctcttgcagtgaggtgaagacatttgaaaatcaccccactgcaaactcctccccctgctagaaacctcacattgaaatgctgtaaatgacgtgggccccgagtgcaatcgcgggaagccagggtttccagctaggacacagcaggtcgtgatccgggtcgggacactgcctggcagaggctgcgagcatggggccctggggctggaaattgcgctggaccgtcgccttgctcctcgccgcggcggggactgcaggtaaggcttgctccaggcgccagaataggttgagagggagcccccggggggcccttgggaatttatttttttgggtacaaataatcactccatccctgggagacttgtggggtaatggcacggggtccttcccaaacggctggagggggcgctggaggggggcgctgaggggagcgcgagggtcgggaggagtctgagggatttaagggaaacggggcaccgctgtcccccaagtctccacagggtgagggaccgcatcttctttgagacggagtctagctctgtcgcccaggatggagtgcagtggcacgatctcagctcactgcaacctccgcctcccgggtttaagcgagtctcctctctcagcctcccgaatagctgggattacaggcgcccaaccaccacgcccgcctaatttttgtatttttagtagagacgggttttcaccattttggccaggctggtctcgaaccccgacctcaggtgatctgcccaaaagtgctgggattacaggcgtcagccaccgcgcccggccgggaccctctcttctaactcggagctgggtgtggggacctccagtcctaaaacaagggatcactcccacccccgccttaagtccttctgggggcgagggcgactggagacccggatgtccagcctggaggtcaccgcgggctcaggggtcccgatccgctttgcgcgaccccagggcgccactgccatcctgagttgggtgcagtcccgggattccgccgcgtgctccgggacgggggccaccccctcccgcccctgcccccgcccctttggcccgccccccgaattccattgggtgtagtccaacaggccaccctcgagccactccccttgtccaatgtgaggcggtggaggcggaggcgggcgtcgggaggacggggcttgtgtacgagcggggcggggctggcgcggaagtctgagcctcaccttgtccggggcgaggcggatgcaggggaggcctggcgttcctccgcggttcctgtcacaaaggcgacgacaagtcccgggtccccggagccgcctccgcgacatacacgagtcgccctccgttatcctgggccctcctggcgaagtccccggtttccgctgtgctctgtggcgacacctccgtccccaccttgtcctggggggcgccctcgccccaccagccccgatcaagttcacagaggggcccccggccaccctcaaggcctcggttccttacgaggttgaaacgttgcctcagaatctccccgcccctccttggtctgcagccgagatcttcagccacggtggggcagctatcccccgggaccgaccccctggggtggcctcgcttcttcagaggctgtgaatggcttcggttcagctgtccaagcggcgatttttcctctgggtgaaatggattagattttagatttccacaagaggctggttagtgcatgatcctgagttagagctttttaggtggctttaaattagttgcagagagacagcctcgccctagacaacagctacatggccctttccctcctgagaaccagcctagcctagaaaaggattgggattgcctgatgaacacaaggattgcaggaaactttttttttaattggcaagggggttggctttgactggatggagagctttgaactgccttgaaattcacgctgtaactaacacaccagtttcctctgggaggccagagagggagggagggtgtaatgaaatacggatgattgttcttttatttttatttacttatttattttttaactttttgtagagatgaggtctcgcttggttgctcaggctggtcttgaactcctggcctcaagcgatcctcctacctcagcctcccaaagtgttgggattacaggagtgagccaccgcgccccaccggggatgatgatgattgcaaacattctgccactcagttttacaaaagaaagagaggcactggattaatgtgtatctcactcaccaatcaacctcttccttaagagaaaatgttaaggaagtcttaggcaaggccttgtttgttcatcactttagtttctctctcccgggatggctgagaatgtgatgtttcctctgttgtcaaggagactacacccctgatgttttcctccagacttctgagagctggtgtgtgtttctagcactttctagctgcaccacctcacgctgtagctggcttcaaggcatatccaggggggagtttcttgtccatttcctttacaaagggaagttgttggaatctgaaccgcaagccttcacttagaccaaaatcaggcaacagcggtgagcgcagctccaaacgtgtcaatgactcacccaaatttgagtaagggagttggctgctttaacgagccgcagggtgattcccttgtcatttccggaaatacctatcttccagggaacactgggaaaaaacagggagacctttgttgagacagaaaacctgtaggggaattctgttcctcattcctgctcttatctgtagacttcctccctgataagatccaattctagatgggtcggttgctccttgctttgatgggtgctttgatgggctttattattattattattattattattattattttgatgggctttttgatgtcccttttccttccacactctgtcccaactgtcaagcaaatagccttttgttgctaagagactgcagatgtaaccgaccagcagcaaacagtgagtcaggctctctcttccggaagcaaaatcaattgctgagatcactctggggaaaatacccaccttatttggaaagaagcactgatcaattgatgtctattttttttttttttgagttggagtctcgccctgtcacccaggctggagtgcaatggcataatctcgcctcactgcaatccccgcctcccgggttccagcaattctcctgcctcagcctcctgagtagctggaattataggcgcctgccacaacacccggctaatttttgtatttgtagtagagatggggtttcaccacgttggccaggctggtctcgaactcctgacctcgtgatccacccgcctcagcctcccaaagtccaaggattgcaggcgtgacccactgtgccagccaatcaattgatttctcattcattttcagctggctctgttcccttaagccaggggattttcgtttgtttgtttccccttcaaggaaatgattctagctacagttttgatttccttgtacaactgttttcagtagcacagggaaagaaaacatcgaaagcattcaccacctcatttgtgtgctgggggaaaaagcagaaatgtgtattctctttttttgtttcgatgaccttgttcctgacttgttactcgtgacttgagagatcagagggctagaggactagaatttatagaggtgttttttttgtttgtttatttttgttcgagttgcccaggctggagtgcagtggcgcaatctcggctcactgcaacctctgcctcccaggttcaagcgattcttcggcctcagcctcctgagtagctggaactacaggcgcccgccaccacacccagctaatttttgtatttttcagtagagatgggatttcaccatattggtcaagctggcctcgaactcctgacctcgtgatccacccgcctcagtttcccaaagtgctgggagtacaggcgtgagccgccgtgcccggcctttttgtgtttttgtgtttttgagaggagctcattgctttttaggcttccctagcgtgagaaaatctggggatccatgctctagtttacttcctttttttttttttttttgagatggagtctcgcttagattgcctaatctcagctcattgcaacttctgcctccggggttcaagggattctcgtgtctcagcctcctgggtagctaggatacgggcacccgctaccatgcctggctaattttgtacttttagtagagacagggtttcgccacgttggccaggctggtctcgaactcctgacctcaggtgagccgcctgccttggcctcccaaagtgctgagattacaggcgtgagccaccgcgcttggcctaatttgcttttcctgaaattcaaatggtctaatatgaaaaacgccaaccttgcttgaaagaataagaaagaggtgcggtttcgttgggccgttgatgtttggaacaggactggttttgtccccttgctcggaaagggcagcaactgtgaggacagctccctgacgtgctctcactcagcactgttccgttcctgagcactgtccccactagctaggccaagggagctcatttggcaggcaactgctgtctggctgcgcctgtggcagtaaaatctgcctttattttttggaggcagggtcttgccctgtcgctcaggctgaagtgtgcagttatagctcactgcagcctccagcttctgtactcaactgatcctcctctctcagcctcctgagtagctgggactatacgcacgtgttaccactcccacctcagtttgtttgtttatttatttatttatttatttattgagatggagttttgctcttgctgcccaggctggagtgcaatggcgcgatctcggctcaccgcaacctccacctcctggttcaagcgattctcctgcctcagcctcctgagtagctgggattacaggcatgcaccaccacgcccggctaattttgtatttttcgtagagatggggtttctccacattggttcaggctgttctcgaactcccaacctcaggtgatccacccgcctcagcctcccaaagtgctgggattataggcgtgagcccccgaacccggccactcccagctaagtttaaattttttgtttgtttgttcgtttgtttttattttttgagacagagtctcccgcccaggctggagcgcagatcactgcatccttgacctcccaggcttaagccatcctccccactcagcctcccaagtagctgggattacaggtgtgtgccactatgcttggctaagttgtgtattttttgtagagatggggttcaagggattctcgctttgttgcctcggttggtctcaaactcctgggctcaagcagtcctccctcctcagcctcccaaggtgctggggaaatccacttttgaaacattgtctggagagttgcccaggtggtagatcacagaaataggtcatcgtggggtccttcccatgggtgcagtcttgagccacctgtggccagcaaatatttggagaataatagtcaggggagagcttgaggtccagggaaaggttttgtttttcttcagggaaaggtttttattgttctttatccctccttaaaggaccttcaggtgttactgacattcccggtctacccagtggcacatttagtttgtaagctgggccctcgtacagaggtagggaggtgagagcattggattagtggtcaccaaagctgcggtcacctagtggggtgatcagaggctcctcccttaagatcttgattgccaacgcctctggcccaactttcctttttatttatcgcaagcctcctggaatctcaattgctttttgcccacccggtgtgtcagcacaagaaatgagtcatttcctcctttaagcacagttgaaattgagctgtgagtcagtgaggtgtgtacgatattgtcaaagcggggtgtgtacagtattgacagatctgtagttgggcaagagaattatcagagtttgtgaccacagcagattccaaagctcgactcattttcttctctcttccttcccttttttcttttctttttttttttttttttgacagagtctcgctctgttgcccaggctggagtgcagtggcacaatctgggctcactgcagcccctgcctcctgggttcaaatgattctcatgtttcagcctcccgagtagctgcaattacaggcattcgggttcaagtgattctcctgcctcagccacctgagcagctgggattacaggcgcccgccaccacgcccggctaatttttgtatttttagtagagacggggtttcaccatgttggccaggctggtctcgaactcctgaactcaggtgatccgcccacttcggcctcccaaagtgctgagattacagacgtgagtcaccgcgcccagcctgttctgttctttaattctcaaaacaccctctaggaagtagagactgccattctcccccattttacagatcaggaaactgagtcccagaaggatttagtcagttacccaagttgttctagttaaatggcctggaaagccagtgaagcccaggattgtctatctaacccccttactactctaactttcagggaatccacatgaatgtgctgggtcaaccatcaaagttgaaatggataaagggggctggatgcggtggctgatgcctgtaatcctagcactttgggaggccgagatgggtgggtggattgcttgagcccaagagtttgagaccagcctgggcaacatagtgagacacctgtctctgcaaaaaataaataaaaagttagctgagtgtgatggtgcacccctctagtcacagctgttgagttaggcttaggcaggaggatcgcatgaacctgggaggtggaggcggccgtgagcctcagtcatgccactgcactccaacctgggcaacagagtgaaagccggtgtccgaaagagaaagaaaaaaagacatagatacatcttttaaagttaggttgtatgttaattacctacaactcagtttcaactgtgcttaaaggaggaaatgactcatttcttgctacatatcaaattagcccaaaatgtagtggcttaaaacaacacatttatgatttctcagtttttgcgtgtcaggaatttggaagcagcacagctagacggttccagctcagggtctctcatgaagttgcaatcaaaatattggcaggagagaaaaacatattttcagaagctgcaggcataggaagacttggctggggttgaaggatccacttccaagatggcgcactcagtggctcttggctggaggcctcagttccctgctgcgtggagctctccctccagctgcttgagtggactcatgacatgcagctggcctcccctggagcagtcgatccaacaatgagcatggccatgaactaggctcagaagccactccctgtcgtctctacattttcctatcagaagcaagtcattaaaagtccagtgccactccaggggagacgaattaggctctgccttctgaaaggattatcacagaagatgcggtcctatattctttttttaaaattattcttttttttattttgtagagatggggtcttggtatgttgcctaggccagtctggaattcctgggctcaaacaatcctgtctctgcctcccaaagtgttgggattacaggcatgagccactgcacctggtcatgtggtcatattttctttttcttttttttttttttttgagacagagtctctgtcgcccaggctggagtatggtggcgtgatctcagttcactgcagcctccgcctcccgggttcaagcgattctcctgcctcagcctcctgagtagctgggattacaggcgcccgccaacatgcccagctaatttttttagtagagatggggtttcaccatgttagccaggatggtctcgatctcctgatttggtgatccgcccaccttggcctcccaaagtttcaaccatcgatcagaacttattgatgtacttatgtagctaggcacggtggcgcgtgcctgtaatcccagctacttggaagggttaaggcaggagaatcgcttgaacctgggaggcagaggttacagtgagtcaagatcataccattgcactccagtctgggcaacagaatgagactctgtctcaaaaacaaaaaacaaacccttgtatgtgattttcctggatagcatctgttacatcttcacaaagataaaaagtcagacttggctgggcatggtggctcacacctgtaatcccagcactgagaggctgaggcaggcagatcacttgaggtcaggaatttgagaccaggctgggcagcatggtgaaaccccgtctctacaaaaaatacaaaaattagccgggtgtggtgtcacgcacctgtattcccaagctactcaggaagctaaggcaggagaatcacttgaacccagaggtggaggtttgcagtgagttgagattgtgccattgcactccagcctgggcgacagagtgagactctgtgtcaaaaataaaataaaataaaattttaaaaaaggcagatttttttttcttcttggtattgttaccttattatagtaataataagtgcatagtgcatgctgagataagcaatcataatttgttattgcggccgggcatggtggctccagcctataatcccagcactttggtcaggagttcaaggccagcctggccaatatagtgaaactccatctctactaaaatacaagaaattacctgggcatggtggcagttgctggtgatccccagctacttgggaggctgaggcaggagaatcgcttgaacctgggaagcagaggttgcagtgagccaagattgcaccactgcactccagcctgggtgacagagtgagactctgtctgaaaataataataataataatttgttattgcttttattgccttagtttacatagggaatcaaagtttatactttgatttataaaagttgctttgattctagttcacagaaccagaatctttcatataaaggtattagagggcccagtgtggtggctcatgcctgtaatcccagcatattgggaggctgaggagggaggatcactttaggagtttgaggccagcctaggcaacatagtgagaccttgtctctacaaaaaattccaacattagctgggcatggtggcatgtgcctgtagtcccatttatttggggggctgaggcaggaggatcacttgagcccacgaggttcaatccaggttgcagtaagccatgatcctgccactgcactccagtttgggtaacagagcgaagctatgtctcaaaaaaagaaaaaaaaagtattctaaatccaaatttaatatataaaactaaatgcaggccaagtgtggtggcatatacctataatcacaacactttgggaggctgaggtgggaggattgcttgagcccaagagttcaagaccagcctaggtaacacagtaagaccccatctctacaaaaagtagaaaaattagcctggcatggtggtgagtgcttttaatcccaactacttagggggctgagatgggaagattgcttgagcctcagagtttgaggctgcagtgggccgtgatcgctccactgatcgctctaaagtgagaccctgtctcaaaaaaaaagaaaatagaagaaaactaaatacattcaataagactttgatctcttttccaaggtgtaaatatattttgggaaattttccagttactttgttctcattttaatgtaataatctaagtcttggttttctaaggaaaagttttctcttattatatcttttgttaatgtttctctcccatttcttttgatctgatcttcagatacatgattatcttcactgctaaatttgtgttctctggcctctacatttataatttctcataattctttatctaagtatttcttccctacctactgaagaaaactcaagttttcttccaccttaatgattatgctgtgtctgtgagttttcttcatgactctttacagtacaagttttttgtttttgtttttttaatggtcagatggatagaacaacacaggttttgtttgttttgttttaacttttaaaaaaattataatagataaagggtctcactacgttgtccaggctgatctcatactcctgggctcaagcaatccacccacctctgcctcccaaagtgctgggattacagtcatgagccaacatgcctgggcagtacaggttttttttgagacggagttttgttcttgttgccgaggctggagtgcaatggcacaatcttggctcaccacaaagtctgcctcccaggttcaagtgattctcctgcctcagcctcctgagtagctgggattacaggcatgtgccaccacgcccagctaattttgtatttttagtagagacggggtttcaccatgttggccaggctggtttcgaactgctgacctcaggtgatctgcccacctcggcctcccaaagtgctgggattacaggcatgagccaccatgcccagctgtagtacaggttttaatatgctaaatactcttcctttctttattaatgtgcatggaagttctaatatttttttcccataccccagagagtccatattttggaatcaacaacactagcctttgttgacaagtgtctctcttgggttccttctttgtgtcctccactgaattttggggttcataaaatttcatttgttgtgcttgcttaattccctgggaatcagactgttcctgatcggatgacatttctggttaattctttagttggcaggaaatagacacaggaaacgtggtcagtttctgattctggcgttgagagaccctttctccttttcctctctctcagtgggcgacagatgygaaagaaacgagttccagtgccaagacgggaaatgcatctcctacaagtgggtctgcgatggcagcgctgagtgccaggatggctctgatgagtcccaggagacgtgctgtgagtcccctttgggcatgatatgcatttatttttgtaatagagacagggtctcrccatgttggccaggctggtcttgaatttctggtctcaagtgatccgctggcctcggcctcccaaagtgctgggattacaggcaccacgcctggcctgtgacacgattcttaacccctttttgatgatggcggctggaaaagtggccagtggattttgatgtattcaatcatgaattaggaggtggggagagaatgaattattggagctttccttaaagccattaaatggctctattgttttttcaattgatgtgaatttcacataacatgaaattaaccagctcagtggcattaatacatctgcaatgctgtgtggccaccacctctatcttgttccaaaactttgcataacctaatgtctttttttttttttttttttgagacggagtctcgttccatcacccaggctggagtgcagtggtgtgatctcagctcactgcaacctccgcctcccaggttcacgccatcctcctgcctcagcctcccgagtagctgggactacaggcaccctccaccacatccggctaattttttgtatctttagtagagatggggtttcaccatgttagccgggatggtctcgatctcctgacctcgtgatccacctgcctccgcctcccaaagtgctggcattacaggcgtgagccaccatgcccggcctatttttttttttaagagatggagtctaattctgttgcccaggctggagtccagtggtaccatcatacttcactgcagccttgacctcttgggctcaagtgattctcttgcctcgaactcccaaagtattgggattacaggtgtgagccaccgcactcagcctaatgtccagtttttaacaagctccatttaaatgccctccgttttgacccataaaggggtaggcttggccgggcacaatggcttgtgtctgtagtcccagctacttgggaggctgaggcagaaaggcagaaagattgctttataaagcccaggagtttgagggccacctgggtggcatagctagacctcatctctaaaaaataagtaataaataaatatttgtttttgtttttttctttttcttttcttttttttttttttttgagacggagtcttgctctgttgcccaggctggagtgcagtggcgcgatctcagctcactgcaagctgtgcctcctgggttcatgccattctcctgcctcagcctcccgagtagctgggactacaggcgcccactaccacgcccagctaattttttgtatttttagtagagatggggtttcaccacgttagccaggatggtctcaatctcctgacctcgtgatccgccagctttggcctcccaaagtgttgggattacaggcgtgagccactgagcccgccccatatgtatgtatatatatatttttttaaaatgggagaccaggcatggtggctcatgcctagaatcccagcactttgggaagctgaggtaggcggatcacttgaggccatgagtttgagaccagcctgctcaacatgatgaaacttctatctctactaaaaaaaaaagtgggattaggtcaggcacggtggctcacacctgtaatcccagcactttcagaggccgaggcaggaggatcatgaggtcaggagatcgagaccatcctggctaacacggtgaaaccccgtctctactaaaaaaatacaaaaaattagccaggcgtggtggcgggtgcctgtagtcccagctactcaggaggctgaggcaggagaatggcgtgaacccgggaggcggagcttgcagtgagccaagatcgtgccactgtactccagcctgggcgacagagcaagactctgtctcaaaaaaaaaaaaaaaagtgggattgacattctcttcaaagttctggggttttcctttgcaaagacaggattggcaaggccagtgggtcttttttgtgtgtgtgtgtgtgacggagtctcactctgccacccaggctggagtgcaatggcaggatctcggctcaccgcaacctcctcctcccaggttaaagtgattctcctgcctcagcctcccgagtagctgggactacaggtgcccgccaccacacccaactaatttttgtatttttagtagagacagggtttcactatattggccaggctggtcttgaacccctgacctcacgtgatccacccgccttggcctcccaaagtgctgggattacaggcgtgagccactgtgctcggcctcagtgggtctttcctttgagtgacagttcaatcctgtctcttctgtagtgtctgtcacctgcaaatccggggacttcagctgtgggggccgtgtcaaccgctgcattcctcagttctggaggtgcgatggccaagtggactgcgacaacggctcagacgagcaaggctgtcgtaagtgtggccctgcctttgctattgagcctatctgagtcctggggagtggtctgactttgtctctacggggtcctgctcgagctgcaaggcagctgccccgaactgggctccatctcttgggggctcataccaagcctcttccgcccttcaaatccccccttgaccaggaggcattacaaagtggggatggtgctacctcttcgggtttgtcacgcacagtcagggaggctgtccctgccgagggctagccacctggcacacacactggcaagccgctgtgattcccgctggtcgtgatccccgtgatcctgtgatccccgccccgtgaggctgaacacatagtgacgcttgctagccaagcctcaatgacccacgtaacatgaagggggaaaagccagaaagttctgccaaggagcaaggccaagaatcccgaagggaaatggactttgaagctgggcgtcttcttggctgtcttaatacaagtggcacatccaaatccaaaaccccgaaattcaaagtcttgagcacccgaaattctgaaacgtcttgagcactgacctttagaaggaaatgcttattggagcattttggatttcggatttttaccactgagtgtggagtcctaattaggaaaaaaaccaggctgaccgaaccaaaggaaagcaataaaagaaggcagatagggtcaggcacggtggctcacccctgtaatcccagccttttgagaggctgaggcgggtggatcacttgaggtcaggagttcgagagcagcctggccaacacggtgaaaccccatctctactgaaaatacaaaaactagccaggtatggtggcgtctgcctgtaatcccagctactcgggaggctgagacaggagaatcacttgaacctgggaggcagaggttgcagtgagccaatatcacgccattgcactccagcctgggggacaagagcgaaattctgtctcaaaaaaaaagaagaagaaggccgacaaactatgtaactctgcctttctccatggtccagaacacacagccctcctgcgtaaataactccttatcttcctgctcccagctatcatcagacacctcggctgatagaaaattgcaagttagctcactgcaacctcggcattataagtactgcacaaagccctcttcagcgcacagcacaagcaccattctataaaatctccagcaagcggccaggtgcagtggctcatacctgtaatcccagcattttgggagactgaggcgggcggatcacctgaggtcaggagtttgagaccagcctggccaacatggtgaaaccccgtctctattaaaaatacaaaaaaattagccaggcgtggtggcaggtgcctgtaatcccagctacttggaaggctgaggcaggagaatcgcttgaacccgggaggtggaagttgcagtgagccgagatcttgccatcgcactccagcctgggggacaagagtgagacttcgtctcaaaaaaaaaaaaaaaaattcccagcaagcctttgtcttctggcagtcagctcctctcttgctgacctgctcattgctttcttgcaaggtattttcctacctactttctggaataaatctgtctttctgtacttacaactaccttttttaaaatttctttcttttttgagatggagtctcactctgtttgcccaggctggagttcagtggtgcaatctcagctcactgcaacctctacctactgggttcaagcgattctcctgcctcagcttcccgagtagctgggattacaggcgtgcaccagcacgcaggctaatttttgtatttttagtagagacggggtttcaccatgttggccaaggtggtcttgaactcctgacctcaagtgatcctcccacctcagcctcccaaagcgctaggattacggccatgagccactgaggccggctgcacctacaactgtcttgataaattcttacccccacaccactggtccagatagtcagtgctcacccacaacattaaggatattccaaatttgaaacattccaaaatcagaaaaatattccaactctgaaaatattccaaaatccaaaaaaattcaaaatccaaaacacttctggtcccaagcattttagagaagggatactcaacccaaaataaggacagcaattctataaattgtgctaccatcttgcaggtctcagtttaacagctttacacctattagcgcaccagtgctcatagcagtgctgggaaatgtgtacagatgaggaaactgaggcaccgagagggcagtggttcagagtccatggcccctgactgctccccagcccgcctttccaggggcctggcctcactgcggcagcgtccccggctatagaatgggctggtgttgggagacttcacacggtgatggtggtctcggcccatccatccctgcagcccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcggtatgggcggggccagggtgggggcggggcgtcctatcacctgtccctgggctcccccaggtgtgggacatgcagtgatttaggtgccgaagtggatttccaacaacatgccaagaaagtattcccatttcatgtttgtttcttttttttcttttctttctttattttgtttttgagatggagtctcactctgtgatttttttcatctctaaatttcctacatccatatggccaccatgaggccccaggctggccgatggttgctgttagcttattgggaaatcactgtttggaaggtgctggttgttttttgttgtttgttgtttttgtttttgtttttgttttgagacggagtctcgctctgtcgccagggtggagtgcagtggcgcgatcagctcactgcaacctccgcttcctgggttcaagccattctcctgcctcagcctcccaagtagcgcggattacaggcatgtgccaccacctccggctatttttttttctatttagtagagatggggtttcaccatgttagtcaggctggtcatgaactcttgacctcaggtgatccacccgcctcggcctcccaaagtgctgggattacaggcgtgcactgctgcacccagcctttttttgtttttttgagacagggtcttgctgtcacccaggttgaagtaaggtggcacgattatggctcactgcggccttgatctccttggctcaagcgatcctctcacttcagcctctcaagcagttggaaccacaggctgtaccaccaagcctggccaatttttttgtacagacacaggctggtcttgaactcctgggctcaagcaatcctcctgccttggcctcccaaagtgctgggattccaggcatgagccgctgcacccggcaaaaggccctgcttctttttctctggttgtctcttcttgagaaaatcaacacactctgtcctgttttccagctgtggccacctgtcgccctgacgaattccagtgctctgatggaaactgcatccatggcagccggcagtgtgaccgggaatatgactgcaaggacatgagcgatgaagttggctgcgttaatggtgagcgctggccatctggttttccatcccccattctctgtgccttgctgcttgcaaatgatttgtgaagccagagggcgcttccctggtcagctctgcaccagctgtgcgtctgtgggcaagtgacttgacttctcagagcctcacttccttttgttttgagacggagtctcgctctgacacccaggctggagtgctgtggcacaatcacagctcacggcagcctctgcctctgatgtccagtgattctcctgcctcagcctcccgagtagctgagattaaaggcgtataccaccacgcccggctaattttttgtatttttattagagacagggtttctccatgttggccaggctggtcttgaactcctggtctcaggtgatccacccgcctcggcctcccaaagtgctaggattacaggtgtgagccactgcgccaggcctaatttttttgtatttttagtagagatgcggttttgccatattgcccaggctggtctcgaactcctgggctcaagcgatctgcctgccttggcctcccaaagtgctgggattacaggcacaaaccaccgtgcccgacgcgttttcttaatgaatccatttgcatgcgttcttatgtgaataaactattatatgaatgagtgccaagcaaactgaggctcagacacacctgaccttcctccttcctctctctggctctcacagtgacactctgcgagggacccaacaagttcaagtgtcacagcggcgaatgcatcaccctggacaaagtctgcaacatggctagagactgccgggactggtcagatgaacccatcaaagagtgcggtgagtctcggtgcaggcggcttgcagagtttgtggggagccaggaaagggactgagacatgagtgctgtagggttttgggaactccactctgcccaccctgtgcaaagggctccttttttcattttgagacagtctcgcacggtcgcccaggctggagcgcaatggcgcgatctcggctcactgcaacctctgcctcccaggttcaagtgattctcctgcctcagcctcctgagtagctgggattacaggcgcccaccaccaagcccgggtaattttttgtatgtttagtagagatggggtttcactatgttggccaggctggtgttgaactcctgacctcatgatccgcccacctcggcctcccaaagtgctgggattacaggcgtgacccaccccatgaaaaaaaattaaaaaatgaagcgatgctgggcgcggtggatcacgcctgtaatcccagcactttgggaagctgaggcaggcagatcacgagggcaggagattgagaccatcctggctaatacggtgaaaccccatctctactaaaactacaaaaaattagccgggtgtggtggcaggcacctgtgatcccagctactcaggaggctgaggcaggagaatcgcttgaacccaggaggtggaggttgcagtgagccgggatcacaccattgcactccagcctgggtgacagagtgagactctgtctcaaaaaaaaaaaaaaaaaaaaaagcgaattctgaaatacatgaattcttttccttagatgcctgcttctgtcttgaggtttgttgttgttatttcgaaacagagtcttgctctgtcgctcaggctggagtgcagtggcatgatcttggctcaccacaacctccggctcccaggttcaagcgattcttctgcctcagcctcctgagtagctgggattacagctgaatgccaccttgctgggctaatttttgtatttttagtagagatggggtttcaccatgttggccaggctggcctcgaactcctgacctcgagtgatctgcccgcctcctgaagtgctgggattacaggcgtgagccacctcgtcctggtgagggtttttttttttccccaaccctctgtggtggatactgaaagaccatattaggataactgtacagtatagagaaggcagtggcaagttttctctgtcatataccagagtgggcttgggcatggtggcatactcctgtagtctcagctaatcaggaggctgaggaaggaggatcgcttgggcccaggagttggagactgtagtgagctgtgatcacaccaccacacttcaatctgggcaacagagcaagagaccctatctctaaaaaaaagtaagtatttcggacactgtgggccatacggtctctggtgcagtttctcaacatggctgttgggtgaacacaaccacgcacagaacgcaaaccaatacacgtggctgtgggcccagaaaatgttatttatggacacaaaaattggaatttcatataactgttttgtgtcatgaaaatgatttccctttttatttttatttttcttctcaagtatttaaatatgtaaaagccatttttaggcctggcaggatggttcacagctgtaatcccagcactttgggaggtcgaggcgggaggatcacgaggtcaggagatcgagaccatcctggccaacacagtgaaaccccgtctctactaaaaatacaaaaaattaaccaggcttggtggcgcgcgtctgtagtcccagctgctcaggaggctgaggcaggagaatcgcttgaatgcaggaggcggaggttgtagtgagccgaggttgcaccactgcactccagcctgagcgacagagtgagagtccgcctcaaacaaaaaaatgtttgcccatgctggtcttgaactcctgggctcaagctatctgcctgccttggtctcccaaagttctgggattacaggcatgagctacagcgcccggacttttgttgttttatatctatatatctatatataacttgttttatgtatatatataacttgttttatatatatacataaactgcagtaaaaaacatgtaacataaaatttaccttctcaaaccttattaagtgcacagttctgtgccattagcaaattcacactgttgtacaacatcacaaccaccatctccagaacttttttttttttttttattctttttgagacagagtctcactcgtcgcacgggctggagtgcagtggtgcgatctcggttcactgcaacctccacctaccaggttcaagcaattctcctgcctcagccccctcagtagctgggattacaggtgcccgtcctaccacgcccagctaatttttgtattttcagtagagactgactgggtttcaccatgttggccaggctggtctcgaactcctgacctcaagtgatcctcccacctcagcctcccaaagtgctgggaatacaggcatgagccactgcgcccggccccagaactcttttatcttcccaaactgaagctctgtccccatgaaacactcactctccatcccctccccaactcctggcacccaccattctactttctgtccctatgaatgtgatggctctagggacctcctctgagtggaatcagacagcattttccttttttgactggcttatttcactgagccaagtgcggtggcacacgcctgtaatcccaaaactttgggagaccgaggcgggcgcatcacctgaggtcaggagttcgagaccagcccggccaacatggtgaaaccccatctctagtaaaaatacaaaaaattagcctgtcatggtcgtgggtgcctgtaatcccagctaagtgggaggctgaggcaggagaatcgcttgtacccaggaggcggaggtcgcagtgagccgagatcgtgccattacactccagcctgggcaacaagagtgaaactccgtctctcctaaaaatacaaaaaaattagctgggcatggtggcacatgcctgtagtcccagctacttgggaggctgaggcaggagaatcacttgaacccgggaggtggaggttgtaatgagccaaggttggcggcgaagggatgggtaggggcccgagagtgaccagtctgcatcccctggccctgcgcagggaccaacgaatgcttggacaacaacggcggctgttcccacgtctgcaatgaccttaagatcggctacgagtgcctgtgccccgacggcttccagctggtggcccagcgaagatgcgaaggtgatttccsggtgggactgagccctgggccccctctgcgcttcctgacatggcaaccaaacccctcatgcctcagtttccccatctgttaagtgtgcttgaaagcagttaggagggtttcatgagattccacctgcatggaaaactatcattggctggccagagtttcttgcctctggggattagtaattaagaaatttcaggccgggtgcgtaatccctgtaatcccaacaccttgggacgccgaggcgggcagatcacctgaggtcgggagttccagaccagcctgaccaacatggagaaaccccgtctctactaaaaatacaaaattagccgggcttggtggtgcatgcctataatcccagctactcaggaggctgaggcaggagaatcacttgaacctgggaggtggaggttgtggtgagccaagatcgtgccattgcactccagcctgggcaacaagagtgaaactccatccaaaaaaaaaagaaaagaaaagaaaaaaaagaaaagaaatttcagctgacacagcttcacactcttggttgggttcccgtggtgaatgatgaggtcaggtgatgactggggatgacacctggctgtttccttgattacatctcccgagaggctgggctgtctcctggctgccttcgaaggtgtgggttttggcctgggccccatcgctccgtctctagccattggggaagagcctccccaccaagcctctttctctctcttccagatatcgatgagtgtcaggatcccgacacctgcagccagctctgcgtgaacctggagggtggctacaagtgccagtgtgaggaaggcttccagctggacccccacacgaagrcctgcaaggctgtgggtgagcacgggaaggcggcgggtgggggcggcctcaccccttgcaggcagcagtggtgggggagtttcatcctctgaactttgcacagactcatatcccctgaccgggaggctgtttgctcctgagggctctggcaggggagtctgccgccctgttaggacttgggcttgccagggggatgcctgcatatgtcctagtttttgggaatatccagttaacggaaccctcagccctactggtggaacaggaaccggctttcctttcagggacaacctggggagtgacttcaaggggttaaagaaaaaaaattagctgggcatggtgccacacacctgtggtcccagctactcagaaggctgaggcgggaggattgcttgagggcaggaggattggttgatcctcccacctcagcctccggagtagctgggacctcaggtgcatgccactatgcctggctaattttcttttttctttttttttttttttcgagacggagtctcgctctgttgcccaggctggagtgcagtggcaggatctcggctcactgcaagctccgcctcccgggttcacgccattctcctgcctcagcctccccagtagctgggactacaggagcccgccactgcaccaggccaatttttttgtatttttagtagagacggggtttcactgtgttagccaggatggtctcgatctcctgacttcgtgatccgcccacctcggccttccaaagtgctcggattacaggcgtgagccactgcgcccagccgctaattttcatatttttagtaaaaacagggtttcaccatgttggccaggctagtcttgaactcctgaacccaagtgatcctcctgccttggcctcccaaagtgctgggattacagacaccacacctggctattattattttttagagacagggtgctgctctatcttccagcctgtagtgcagtgcagcctccatcatagctcgctgcagccttgacctcctgggttcacgtgatcgtcccgcctaagcctctggaggagctgggagtactggcatgtgccaccatgcctggttaatttttttttttttttttttgagacagagtctcattctgtcacccaggctggagtgcggtggtgcgatcttggcttactgaaacctccacctcccaggttccagcaattctcctgcctcacccttctgagtagctgggattacaggttccggctaccaaacctggctagtttttgtatgtttagtagagacagggtttcaccatgttggtgaggctggtctcgattctcccgcctcagcctcccaaagtgctgggattacaggcttgagccaccgtgcctggcttttttttttttttttttttttgtggcaataaggtctcattgtcttgcccaggctagccttatgctcctagcctcaagtgatcctcctccctcagcctcccaaagtgctgggattacaggtgggcgccactgtgcctgttcccgttgggaggtcttttccaccctctttttctgggtgcctcctctggctcagccgcaccctgcaggatgacacaaggggatggggaggcactcttggttccatcgacgggtcccctctgaccccctgacctcgctccccggacccccaggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcaggtgagcgtcgcccctgcctgcagccttggcccgcaggtgagatgagggctcctggygctgatgcccttctctcctcctgcctcagcacccagcttgacagagcccacggcgtctcttcctatgacaccgtcatcagcagrgacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatgggtgcgtatccacgacgctgagggctgcagagggaatggagggagcaggaaggagcttcaggaactggttagtgggctgggcatggtggctcaaagcacctgtaatcccagcactttgggaggccaaggtgggtggatcatcaagaccagcctgaccaacatggtgaaacctcgtctctactaaaaatacaaaaattagccgggtgtggtggtgggcacctgtaatcccagctgctcgggaggctgaggcaggagaatcacttgaacctgggagatggaggttgcagtgagccaagacagccccactgcactccagcctgggtgacagagtgagactccgtctcaaaaaaaaaaaaaaaaactaaacaaaaaactggttagtggctagacaacaggatggtatcttccaagcccatggctgactcagcagctcctgggtcaagacactgtgacctgtgtcccctggcaggaagcatcgcccctgccacctgcccggtgtactctgtacctgtcaggtgacatctgctacctaagcacgtgagaggtggcatttcacagtttcagtgtggtgctgacaacccgggacgcacactgtccttgcagctacaatcaggaggtgaatgttgggtttccagcagagaacactggagaaggcacacttggtgtctggaagggaaaagcagggaagagagcatcatcagatgcctgcgggtgaaggtgggcccgctatggccagcgtccctttttatttttatttatttatttatttgagatggaatctcgctctgtcgcccagactgtagtgcagtggtgcgatcacggctcactgcaagctccgcctcacaggttcacgccattctcctgcctcagcctcccgagtagctgggactacaggcacccgccaccacgcccggttaattttttgcatttttattagagacggggtttcaccgcgttagccaggatggtctaaatctcctgaccctgtgatccacccgcctcggcctccctaagtgcttggattacaagcgtgagccaccacgcccggccccctttttattttttattttttgagacggagtctcgctctgtcgcccaggctagattgcagtggcgtgatctcggctcactgcagcctccgcctcccaggttcaagtgattctcctgcctcaacctcccaactaattaggattacaagcatgtaccaccatgcctgactaattttttgtatttttagtagagactgggtttcaccatgttggctaggctggtctcgaacccttagcctcaagtaatctgcctgcctcagcctcccaaacagcggggattacaggcatgagccactgtgcccaacccaaccctggatctcttttaaacaagacaatgctcgctgttgccacagaacaatgggtggggtacatgtggcccagtgtgtttggccacataactgccaggccagagggaaagagactctcagactgtctccactcagatacaaatgtgtgtgttgtgtgcgtgtgttctggtctcatatttgtttgttttgagacagggtgtcgctctgtcactgagtctggagtgcagtggcgcaatcagagttcactgcagcctcaaactcttgggctcagttgattctcccacttcagcctcccaagtagctggaactacaggtgaacaccactgtgcccagctaatttattttatttttagtagagatgaggtctcactatgttgcccaggctggtcttgacctcctagcctcaagcaatcctcctgccttggtctcccaaagtgctgggattacacgtgcgagccattgcgcatggcttgtgttcttgtgtttcttcctttttctttcgagatggcgtctcagtctgccacccaggctggagtgcagtggtgtgatcatagctcactgtagcctcaacttcctgggctcaagcaatcctcttgatttcagcctcccgggcctggccagcatggtgaaaccccgtctctactaaaaatacaaaaatgtagccaggcgtggtggtgggcgcctgtaatcccagctacaccagaggctgaggcaggagaatcgcttgagcctggaaggtggaggttgcagcaagccaagatcgtgccactgcactccagcctgggcaacagagacagactctgtctcaaaaaaaaaaaaaaaaaacccaaacaagccacatttggagtttggggttcccagcaggactatttcccaagcctgagcctggctgtttcttccagaattcgttgcacgcattggctgggatcctcccccgccctccagcctcacagctattctctgtcctcccaccagcttcatgtactggactgactggggaactccygccaagatcaagaaagggggcctgaatggtgtggacatctactcgctggtgactgaaaacattcagtggcccaatggcatcaccctaggtatgttcgcaggacagccgtcccagccagggccgggcacaggctggaggacagaygggggttgccaggtggctctgggacaagcccaagctgctccctgaaggtttccctctttcttttctttgttttttctttttttgagatgaggtcttggtctgtcacccaggctggagtgcactggcgcaatcgtagctcactgcagcctccacctcccaggctcaagtgatcctcctgcctcaccctcctgagtagctgagattacagacacgtgccaccacggcagactaattttattttatttttgggaagagacaaagtcttgttatgttggcctggctggtctcaaactcagggtgcaagcgatcctcccgcctcagccttccaaactgctgggattacaggcgtgggccaccgtacccagcctccttgaagtttttctgacctgcaactcccctacctgcccattggagagggcgtcacaggggaggggttcaggctcacatgtggttggagctgcctctccaggtgcttttctgctaggtccctggcagggggtcttcctgcccggagcagcgtgkccaggccctcaggmccctctgggactggcatcagcacgtgacctctccttatccacttgtgtgtctagatctcctcagtggccgcctytactgggttgactccaaacttcactccatctcaagcatcgatgtcaaygggggcaaccggaagaccatcttggaggatgaaaagaggctggcccaccccttctccttggccgtctttgaggtgtggcttacgtacgagatgcaagcacttaggtggcggatagacacagactatagatcactcaagccaagatgaacgcagaaaactggttgtgactaggaggaggtcttagacctgagttatttctattttcttctttctttttttttttttttttgagacagagttttgctctcgtttcccaggctggagggcaatggcatgatctcggctcaccgcaacctccacctcccaggttcaagtgattctcctgtctcaggctccccagtagctgggattacaggcatgcaccaccaccatgcccggctaattttgtatttttagtagagacggagtttctccatgttggtcaggctggtctcgaactcccgacctcaggtgatctgcctgcctcggcctcccaaagtgctgggattacagacttgagccaccgcgcccagctatttctgttttctttctttcttcttcttctttttttttttctaagagacaggatctcactctgtccccaggcaggagtgcagtgctgtgatcatagctcactgcagccttaacctcctgggctcaagtgatcttcccacctcagcctcccaagtagctggaactacaggtgcacaccaccatgcccagctcatttttgtattttttttttttttgagacagtctcgttctgtcaccccggctggagtgcagtggtacaatcttggctcactgcaacctctgcctcccaggttcaagcgattctcctgcctcagcctcctgagtagttgagattacaggcatgtgtgccatcatacctggctgatttttgtatttttttttagagatggggtctcagtatgttgaccaggcttgtcttaaactcccggcctcaagtgatcctcccacttcagtctcccaaagtgctgggattacaggcatgagccactgcggccggtttgttttcttttttttttcgttttttggagacggaatttcacctttgttgcccaggatggagtgcaatggcacgatatcgcctcaccacaacctctgcctcctgggttcaaaccattttcctgcctcagccttcttagtagctgggattacaagcatgtgccaccacgcccggctgattttgtatttttagtagagatggggtttctccatgttggccaggctggtctcgaactcctgacctcaggtcattcgcccacctctgcctcccaaagtgctgggattacaggcgtgagccaccgtgcccggtggtttgtattctttttactgagagtcgtgaaaggcagtgatcctctgtcacatgtgatcttggctctcaggggacatttggcaatttctagagattttttggttgtcacaagtcaatggggaagactgttggcatttagtgggtagaggctggtgacgctgctgaacacccagaacagggaagtagcaggccctagatagagccatcgtggggaaaccctgctctaaggaaatggcgctattttataaccccacgttcctggcatgattaccaacagccaaaagtggagtccccccaagtgtgttcgtccatttgcattgcagtaaaggaatagctgaggccgggtaatttataaagaaaagagatttaaactgggtatggcagtttatgcctataatcccagaactttgggaggctgaggcaggaggatcgcttgagtccaggagtgtgagaccgagaccagcctggccaacatgacgaaactctgtctctacaaaaaatacaaaaagtaggccaggcacggtggttcacgcctgtaatcccagcactttgggaggccgaggcgggcggatcacgaggtcaggagatcgagaccatcctggctaacacggtgaaaccccgtctctactaaaaatacaaaaacaaaattagccgggtgtggtggcaggcgcctgtagtcccagctactcgggaggctgaggcgggagaatggcgtgaacccgggaggcggagcttgcagtgagccaagatcgcgccactgcactccagcctgggtgaccgagttgagactccgtctcaaaaaaaaaaaaaaaaaaaaaaatacaaaaagtagccaggtgtggtggcaggcacctgtaatcctgggttctcgagaccgaggcatgagaattgcctgaccccaggaggtggaggctgcagtgagccaagatcatgccactgcactccagcctgggcgacagagtgggactctgtctcaaaaaacaacaaaaaaaaagttctggaaatggatggtggtgatggtgatacttccacaacagcgtgaatctgcttaaggccaccgaactgtgcactcacaaatagtcgagatggtacattttatgttatgtgtatttcaccacaattaaaaactagttgtgggccaggtgtggtggttcatgcctgtaatcccagcactttgggaggtcagagggaggtggatcatgaggtcagcagttcgagaccagccaggccaacatggtgaaaccccatctctactaaaaatacaaaaattagccaggcgtggtggcacatgcctgtagtcccagctacttgagaggctgaagcaggagaatcgcttgaacctgggaggctaagattgcagtgagccgagatcgtgccactgcactccagcctggacgacagagtgagacttcgtctcaaaaaaaaaaccaaaaaaaaaattagctgtgggtcaggcactgtggctcacgcctgtaatcccagcactttgggagaccgaggtaggtggatggcctgaggtcaggagttcgaatccagcctggccaacatggtgaaagcccgtctctactaaaaatacaaaaaattagtcaggtatgttggcacacctgtaatcccagctactcgggaggctgaagcaagagaatcgtttgaacccaggaggtggacgttgcagtgagccgagattgggccactgtactccagcctgggcaacaaaagtgaaactctgtctgaaacaaacaaacaaacaaacaaacagacaaacaaaaaaactagttgtggagagagggtggcctgtgtctcatcccagtgtttaacgggatttgtcatcttccttgctgcctgtttaggacaaagtattttggacagatatcatcaacgaagccattttcagtgccaaccgcctcacaggttccgatgtcaacttgttggctgaaaacctactgtccccagaggatatggtyctcttccacaacctcacccagccaagaggtaagggtgggtcagccccacccccccaaccttgaaacctccttgtggaaactctggaatgttctggaaatttctggaatcttctggtatagctgatgatctcgttcctgccctgactccgcttcttctgccccaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacaggtgtggcacacgccttgtttctgcgtcctgtgtcctccaactgccccctcctgagcctctctctgctcatctgtcaaatgggtacctcaaggtcgttgtaaggactcatgagtcgggataaccatacttttcttggatggacacatcagcaccgggcttgacatttacccagttcccctttgatgcctggtttcctctttcccggccccctgaagaggtgatctgatttctgacaggagccctgagggaggaaatggtcccctttgttgacttttctttttctttatttttttcttttgagatttgctgtcacccagcctggaatgcagtggtgccatcttggctcactgctacctctcccactgggttcaagcaattctcctgcctcagcctcccaagtagctgggattacaagcatgcgccaccatgcctggctaagttttgtatttttagtacagacagggtttctccatggtggccaggctggtcttgaactcctgacctcaggtgatcctcccacctctgcctcccgaagtgctacgattacaggcatgagccaccgcgcccatccccctttgttgacttttctcatcctctgagaaagtctcagttgaggccagcacctccctcaagtgaattgaatctcccttttgaacaacaacaaataacaatatgacccagacgtggtggctcacacctgtggtcccagctactcgggaggctgaggtgtgaggattgcttgagcccaggaggtcaaggctacagagagctataatcacaccacttcactccagcctgggggacaaagtgaaaccctgtctgaaaaaaacaaaaaaagaaaaaggaaaaagaaacaatacgatcacaaagtagatattcatagtgtttattttcagtactcttttttttttttttttttttttttgagacggagtcttgctctgttgcccaggctggagtgcagtggcacgatcttggctcactgcagcctctgcctcccaggttcaagcgcttggctcactgcaacctccgcctcctgggttcaagcgcttcttctgcctcagcctccccagtagctgggactataggcacgtcccactacgcccagctaattttttgtattttttagtagagatggggtttcactatgttagccaggatggtctcgatctcctgacctcgtgatctgcctgccttgggctcccaaagtgttgggattatgggcatgagccactgcacctggccttttttttttttttttttgagatggagtttcgctcttgttgcccaggctggagtgcaatggtgtgatctcggctcactgcaacctctgcctcctgggttcaagcaattctcctgcctcagcctcccgagtagctgggattacaggcacctgccaccacgcctggctaatttttgtacttttagtagagacggggtttctccatgttggtcaggctggtctcaaactcctgacctcaggtgatccacccacctcggcctcccaaagttctgggattacagacatgagccaccgcgcctggccgtgtctggccttttttagttatttcttttttttttttttttttttttgagacagagtcttactccgtcgcccaggctggagtgcagcggtgcgatgtctgcgcactgcaagctccgccccctgggttcatgccattctcctgcctcagccttctgagtagctgggactgcaggcgcctgccactacgcccggctacttttttgtatatttagtagagatggagtttcactgtgttagccaggatggtctcgatctcctgactttgtgatccgcccgcctcggcctcccaaagtgctgggattacaggcgtgagccaccatgccaggcttttttttttttttttttttttgagacggagtcttgctctgtcgcccaggctggagtgcagtgccatgatctcagctcactgcaagctccacttcccaggctcacgccattctccagcctcagcctcccaagtagctgagactacaggggcccgccaccacactcggctaatttttttgtatttttagtagagacggggtttcaccatgttagccaggctggtcttgaactcctaacctcaggcgattcacctgcctcggcctcccaaagtgctgggattaaaggtatgagccacctcgcctggtgtgagccacctcgcccagcctgagccacctcacccagcctaagccactgtgcctggcctgattttggactttttaaaaattttattaataattatttttgggtttcttttttttgagacagggtcttactctgtcatccaggccatcctgtctgtctgtcatcccagtgatgggatcataccttgctgcagcctctacctcctgggctcaagcgatcctcccccctcagcctcctgagtagctgggagtacaggtgtgcaccaccacacctggctaatttttttttttttttttgtatatagagatggtattttgccatgttgaccaggctagtcttaaactcctggactcactcaagagatcctcctgccttggcctcccaaggtcatttgagactttcgtcattaggcgcacacctatgagaagggcctgcaggcacgtggcactcagaagacgtttatttattctttcagaggctgaggctgcagtggccacccaggagacatccaccgtcaggctaaaggtcagctccacagccgtaaggacacagcacacaaccacccgrcctgttcccgacacctcccggctgcctggggccacccctgggctcaccacggtggagatagtgacaatgtctcaccaaggtaaagactgggccctccctaggcccctcttcacccagagacgggtcccttcagtggccacgaacattttggtcacgagatggagtccaggtgtcgtcctcactcccttgctgaccttctctcacttgggccgtgtgtctctgggccctcagtttccctatctgtaaagtgggtctaataacagttcttgccctctttgcaaggattaaatgggccaaatcatatgaggggccaggtccttcaggctcctggttcccaaagtcagccacgcaccgtgtgggtcccaaaattttatcaaggcacattcgttgcctcagcttcaggcatctgcccaaaaaggccaggactaaggcaaggagagggagggattcctcagtactcagcttttcacagaggctccaaaaggctaaggaatccagtaacgttttaacacaattttacaatttttttttttgagacggagttttgctcttgttgcccaggctggagtgcagtggcacgatctcggctcactgcaacctctggctcccgggttcaagcgattctcctgcctcagtctcccgagtagctgggattacaggcatgcgccaccacgctcggctaattttgtatttttagtacagaaggggcttctctgttggtcaggctggtcgtgaactctcaacctcaggtgagccacccgcctgagcctcccaaagtgctgggattacaggtgtgagccaccacgcctggccttttttttgagacagagtctcgctctcgcccatgctgtactgcagtgacgcagtctgggctcactgtaacctccgcttcccaggttcaagtgattcttctgccgcagcctcccatgtagagtagctgggattacaggcacccgccaccatgcctggctaattcttgcatttttagtagagatggggtttcacagtgttggccaggctggtctcaaacttctgacctcaagtcatctgcctgccttggccctgccaaagtgctgggattatagatgtgagccaccgcgcctggcctacagtttattctttggtggctcacacctgtaatctcagcactttgggaggccaaggtgggagaatggcttgagcccaggagttcaagtccagcctgggcaacatagcaagaccctatctctactacaaaataaataataaataaactaattttttttcttttaaaacccaactattcaacatggcaatgcaatatattaaaaaaattttttttttctttgaaacggagtctctcactgtcacccgggctggagtgcagtgtcgccatcttggctcactgcaacctccgcctcccaggtccaagtgattctcctgcttcagcctcccgagtagctgggattacaggcacccaccaccatacccagctaatatttttgtatttttagtagagatggggtttcactatgttgggcaggctggtctggaactcctgacctcgtgatctgcccgaggatcggcggcctcccaaagtgctggggattgcaggcatgagccaccgtgcccagccaaaacttttttatttttatttttttgggacacggtctcactgtgtaccccagactggagtgatagagtgctgtcatggctcactgcagcctcaacctccctgggctcaggtgatcttcctgcttcagtctcccaggtagctgggactacaggcatgagccaccacacccagctaatttttgaatttttttgtagagacagggtttcaccttgtggcccagacttgtctctaactccagggctcaagcgatctgcccaccttggcctcccaaagtgctgagattaatgcaatttaaaaaattttttggccaggcctggtggctcatgcctgtattcacaacaccttgggaggcaaaggtgggcagatcacttgaggtcaggagttcgagactagcctggccaacatggtgaaaccccctgtctactaaaaaaatacaaaaattacctgggcacagtggtgggtgcctgtaatcccagctacttgggatgctgagggtggagaattgcttgaacctgggaggcagaagttgcagtaagccaagatcatgccactggactccagcctcagtgacagagcaaaactctgtctccaaaaaaattgttttttttttttttttttcaaatcatcacactacagccaaggcctggccacttacttttgtaaataaagttttattggagccagtggaccagtgaggccgaatcttgcaggtgtaagatcacagtctatccttgaaaattttgatattttgttcattgggtggtttttcattaatttaaattttaaaaaataacatattaaaggctggtgtggaggtgcacgcctgcagtcctagctactcccagaggctgaggcgggagacttgcttgagcccaagagttgaagtccagcctgggcaacatagcgagacccccatctctaaaaataaaaataatgcattagaatattattggattcctgggcagggcacagtggctcacacctgtaatcccagcactttgggaggctgaggtgggtggatcacctgaggtcaggagtttgagaccagcctggccaacatggtgaaaccccgtctctactaaaaatacaaaaattagccaggcgtggtggcaggtgcctgtaatcccagctactcgggaggctgaagcacgagaatcgcttgaatccaggaggcggaggttgcagtgagctgagattgcgccattgcactccagcctggaggacaagagtgaaactccattcccctctgcaaagaaaaggaatattatcagattcctaagctttttggctccccctttagtttgggggctggggtggtgagtgtctgacctggcctcactgtcctccctggatgtgatgagacccaggtgtgggtcaggatgtcattcgtttgtccaccagagggcgcccaaactgctttgagctgctgggaaatggtgctcctagacttttagcaaacaaacaaaaaaaaatggcacatcggcaaatttcagaccattctttttttttttttttttggttccagagtagctgaaatctttgttcagttacaagcaggataaaatggaaactgcctgggagaggctgagaaaccttcttgcttgggggaggtggggcactgctagaattaatcgcttcacagaccagcccatccaggactcctcaaatttggcaaaaaagccattcattcattcattcatttatgtagagacgagggggatctggctatattgcctagattggtctcaaattcctggcctcaagtgatcctcctgccttggtctactaatgtgctgcgattacaggcatgagccaccgtgcctagctctagtggacttgaaatgttgccttgcccagggcccttatgttgaatggcccaggtccacttgtatggttctgtaccaaggttaaccccatcccataatgcctgggacagttgatgcaggacaatcagcttctgtgccattcaacctcaggactgagcatgctgggcattgtggggtccgaaggtggctcccctgtccccttcaaaataccctctttttcttttcttcttttttttttttttttttttttgagacgaagtcttgctctgttgccccagctagagtgcagtggtgcgatctcagctccccgcaacctctgcttcccgggttcaggcgattctcctgcctcagcctcctgagtagctgggattacaggtgcccaccgccacagctggctaatttttgtatttttagtagagacagggtttcaccgtgttggccaggctggtcttgaactcctgacctcaggcaacctgcccacctcagcctcccaaagtgctgggattacaggtttgagccactgggcctggccttttttttttttttttgagagggagtctcactctgttgcccaggctggagtgcaatggcgcgatcttgactcactgcaactccatttcccgggttcaagtgattctcctccctcagcctcccaagtagctgggattacaggtgcatgccaccacggccagctaattttgtatttttagtagagacagggtttcactatgttgatcatgctggtctcaaactcctgaccttaggtgatctgcccgccttagcctcccaaagtgttgggattacaggtgtgagccaccgcgcccagaccaaaatatgctcattttaataaaatgcacaagtaggttgacaagaatttcacctgcaaccttgtcaaccacctagaataaaagcctctgcagccctcccctaaagactcatcaatgtgaggctcaagaaccttcttaggctgggctcggtggctcatttctgtaatccctgcactttggaaggctgaggcaggaggatctcttgaggccaggagttcaagacaagcctgggcaacatagccagacctctgtttctatcccccacaaaaagaaccttcttaaaccggaattgagtcctacaacctcgataactcacaaataagcccgtgtggcctctcrcagacttgggaagttctccaagtgtccagggagatgtgccaggcgctttcctgccgtgaccaccgtcctctgcctgctccatttcttggtggccttcctttagacctgggcctcactcttgcttctctcctgcagctctgggcgacgttgctggcagaggaaatgagaagaagcccagtagcgtgagggctctgtccattgtcctccccatcggtaagcgcgggccggtcccccagcgtcccccaggtcacagcctccygctatgtgacctcgtgcctggctggttgggcctgttcactttttctcctggacagggaacagccccactggtgtcctttatcacccccacggcctctcctggcttggggctgacagtgacaagatcagacagctaaggggtcagatggaggatgtggagctgggtcccgtgctgtggaatagcctcaccgagatttgagtgccttctggggaactggttcccttgcagggggctgtgtggagaggcgcgctctccctgcctcacccatgctcatcctaactcggttaccatcacatctcttttttctttttttcttaaattttaagaaaaaagaaatttaatttttttgagagacagagtcttgctctgtcacccaggctggagtgcagtggcaccatcatgcctcgctgcagcctcaatgtctgggctcaagcgatcctcccacctcagcctcctgagtagctggtgcaagccactataccccacttcctatttcttaaaaagtcacagccctgtgtgtggctaatcctggacagaaatctagaagaagtcagctacttctggggcgtggctcacccagtgggcttcaggttagatatttcttatacttatgaggctgggtgtggtggcttatgcctgtaatcccagcactttgggaggctgaagtgggtggattgcttgggctcaggagttcgagaccaacctgggcaacatggcgaaaccctgtttctagaaaaggtacaaaaattagctgggcaggtggcacgtgcctgtggtaccagctacttgagggcctgaggcaggaggatcgcttgaacctgggaggtcgaggttgcagtgaactgagatcatgtcactgcactccagcctggtgacagagcaagaccccgtctcaaaaaaaaaaaaagaaagaaaaaaattcttatgcatagatttgcctcttttctgtttgtttgttttgagatggagtctcgctctgtcgcccaggctggagtacagtggctcaacctcggctcactgcaacctctgcctcccgggttcaagcaattctcctgcctcagcctcctgagtagctgggactacaggcgcccgccaccatgcccagctaatttttgtatttttagtagagactgactgggtttcatcatgttggccaggctggtctcgaactcttgacctcatgatccgcccgcctcagcctcccaaaatgctgggattacaggcgtgagccaccaggcccaggccgcaaggcgatctctaaacaaacataaaagaccaggagtcaaggttatggtacgatgcccgtgttttcactccagccacggagctgggtctctggtctcgggggcagctgtgtgacagagcgtgcctctccctacagtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcggtgagtgaccctctctagaaagccagagcccatggcggccccctcccagctggaggcatatgatcctcaagggaccaggccgaggcttccccagccctccagatcgaggacagcattaggtgaatgcttctgtgcgctcattcagaatgtcagcggacaatggccttggtggtgtagaggaatgttggataagcaaatagagagctccatcagatggtgacagggcaaagaaagtcaaaaggagttcagaggccgggcgcggtggctcatgcctgtaatcccaggactttgggaggccgaggctggcggatcacctgaagtcaggagtttgagaccagcttggccatcatgacaaaaccccgtctctattaaaaatacaaaaaattagccaggcgtgggagtgggcgcctgtaatcccagctactcgggaggccgaggtagaaaaatcgcttgaacctaggaggcagaggttgcagtgagccgagatcgcgccactgcattccagcccgggaggcaagagcaaaactccatctcaaaaaaaaaaaaaaaaggagttcagaggcccggcatggtggttcacacatgtgatcccagaacttggggaggttgaggcaggagaatcacctgagctcagagttcaagaccagcctgggcagcacagcaagaccccatctctgcaaaaaataaaaatttagcccagtgtggtgatgagcgcctagttccagctactagggaggctaaggcaggaggattgcttgaggctaaggtaggagattgagactgcagtgacttgtgattgcgtcactgcgctccagcctgggtgacagagcaagcccttgtctcttaaaaaaaaaaaaaaattcaaagaagggtttccagagggccaggagggaggaagggagaggaggtgttttatttttttgcttttattttttattttgagacagagtctctctctgtcacccaggttggagtgcagtgctgtgatcttggctcactgcaacttctgcctcctgggttcaagcaattcttatgcctcagcctcagcctcctgagtagctgggattacaacactatgcccgggtaatttttgtatttttagtagagacgaggtttcgccatgttgcccagactggtctcgaactcctgacctcaagtgatccacccgccttggcctccccacgtgctgggattgcaggcgtgagccactgcgcccgccttgatctttacacaaggggtttagggtaggtagccttctctgaaccaggagaacagcctgtgcgaaggccctgaggctggaccgtgcctgttgggtttgaggccgttgtagctggagcaaacagagagaggggtaaaaaggcaggaggctaccaggcaggttgtgcagagccttgtgggccactggggaggactttggcttttgccctgagagcggtgggaagtgactgaatccggtactcaccgtctccctctggcggctcctgggggaacatgcttggggatcaggctgggggaggctgccaggcccagraggtgagaagtaggtggcctccagccgtgtttcctgartgctggactgatagtttccgctgtttaccatttgttggcagagacagatggtcagtctggaggatgacgtggcgtgaacatctgcctggagtcccgtccctgcccagaacccttcctgagacctcgccrgccttgttttattcaaagacagagaagaccaaagcattgcctgccagagctttgttttatatatttattcatctgggaggcagaacaggcttcggacagtgcccatgcaatggcttgggttgggattttggtttcttcctttcctcgtgaaggataagagaaacaggcccggggggaccaggatgacacctccatttctctccaggaagttttgagtttctctccaccgtgacacaatcctcaaacatggaagatgaaagggsaggggatgtcaggcccagagaagcaagtggctttcaacacacaacagcagatggcaccaacgggaccccctggccctgcctcatccaccaatctctaagccaaacccctaaactcaggagtcaacgtgtttacctcttctatgcaagccttgctagacagccaggttagcctttgccctgtcacccccraatcatgacccacccagtgtctttcgaggtgggtttgtaccttccttaagccaggaaagggattcatggcgtcggaaatgatctggctgaatccgtggtggcaccgagaccaaactcattcaccaaatgatgccacttcccagaggcagagcctgagtcacyggtcacccttaatatttattaagtgcctgagacacccggttaccttggccgtgaggacacgtggcctgcacccaggtgtggctgtcaggacaccagcctggtgcccrtcctcccgacccctacccacttccattcccgtggtctccttgcactttctcagttcagagttgtacactgtgtacatttggcatttgtgttattattttgcactgttttctgtcgtgtgtgttgggatgggatcccaggccagggaaagcccgtgtcaatgaatgccggggacagagaggggcaggttgaccgggacttcaaagccgtgatcgtgaatatcgagaactgccattgtcgtctttatgtccgcccacctagtgcttccacttctatgcaaatgcctccaagccattcacttccccaatcttgtcgttgatgggtatgtgtttaaaacatgcacggtgaggccgggcgcagtggctcacgcctgtaatcccagcactttgggaggccgaggcgggtggatcatgaggtcaggagatcgagaccatcctggctaacamgtgaaaccccgtctctactaaaaatacaaaaaattagccgggcgyggtggygggcacctgtagtcccagctactcgggaggctgaggcaggagaatggtgtgaacccgggaagcggagcttgcagtgagccgagattgcgccactgcagtccgcagtctggcctgggcgacagagcgagactccgtctcaaaaaaaaaaaacaaaaaaaaaccatgcatggtgcatcagcagcccatggcctctggccaggcatggcgaggctgaggtgggaggatggtttgagctcaggcatttgaggctgtcgtgagctatgattatgccactgctttccagcctgggcaacatagtaagaccccatctcttaaaaaatgaatttggccagacacaggtgcctcacgcctgtaatcccagcactttgggaggctgagctggatcacttgagttcaggagttggagaccaggcctgagcaacaaagcgagatcccatctctacaaaaaccaaaaagttaaaaatcagctgggtacggtggcacgtgcctgtgatcccagctacttgggaggctgaggcaggaggatcgcctgagcccaggaggtggaggttgcagtgagccatgatcgagccactgcactccagcctgggcaacagatgaagaccctatttcagaaatacaactataaaaaaataaataaatcctccagtctggatcgtttgacgggacttcaggttctttctgaaatcgccgtgttactgttgcactgatgtccggagagacagtgacagcctccgtcagactcccgcgtgaagatgtcacaagggattggcaattgtccccagggacaaaacactgtgtcccccccagtgcagggaaccgtgataagcctttctggtttcggagcacgtaaatgcgtccctgtacagatagtggggattttttgttatgtttgcactttgtatattggttgaaactgttatcacttatatatatatatatacacacatatatataaaatctatttatttttgcaaaccctggttgctgtatttgttcagtgactattctcggggccctgtgtagggggttattgcctctgaaatgcctcttctttatgtacaaagattatttgcacgaactggactgtgtgcaacgctttttgggagaatgatgtccccgttgtatgtatgagtggcttctgggagatgggtgtcactttttaaaccactgtatagaaggtttttgtagcctgaatgtcttactgtgatcaattaaatttcttaaatgaaccaatttgtctaaactcgatgcacgttcttctgttcgcgcgcttctttttgtttttttttttttcctgagatggagcctggctctgtcacccctggctggagtgcagtggcatgatctcggcttactgcaagctccgcctcccaggttcaagcaattctcctgcctcagcctccctagtagctaggattacaggtgagtgccaccacgcctggccaattttttttttttttttttttttgagacagagtctcgctctgtcacccaggctggagtgcagtggtgtgatctcggctcactgcaagctctgcctcccaggttaatgccattctcctgtctcagcctcctgagtagctggggccacaggcgcctgccaccacgcccggctaatttttttttgtacttcttttagtacagacggggtttcaccatgttagccaggatggtctcgatctcctgaccttgtgatccacctgcttcggcctcccaaagtgctgagattacaggcgtgagccaccgcgggtggccaacgctaatttttttgtttttttagatggagtcttgctctgtcgcccaggctggagtgcagtggcgtgatctctgcctactgcaagctccgcctcccgggttcatgccattctcctgcctcagcctcctgagtaactgggactacaggcacccgccaccacgcccggctaattttttgtatttttagtagagacagggtttcaccgtgttagccaggatggtcttgatctcctgaccttgtgatccacccgtctcggcctcccaaagtgctgggattagaggtgtgagccaccacacctggcctagcctggctaatttttgtatttttggtagagacggggtttcaccatgttggtcaggctggtcttgaacttctgacctcaggtaatctgcctgcctcagtctcccaaagtgctgggattacaggtgtgagccaccgcgcctggcctcacttccttctgtcatctgtttgtggattggactccccaggagaaggacccagaaggggaagactcccagaactccgggcaagatgcaatctccgtgggctgccacagtgcctggcaggtgctgtgatggctgagctggtgattgtgttctctgctgtcgcttctctgagttggagattttgtcaagtcccctgctcatccattcatacactcgacaaatatctgttgagtgctaagtgcgaaccatgctctgccgtaggcttgtgggacactacaggggatataagaaatgaaagccgggtgtggtggctcacacctgtaatcctagcagtttgggaggccgaggcgggcagatcatgaggtcaggagatcgagaccatcctagctaacacagtgaaaccccatctctactaaaaatacaaaaaattagccaggcgtggtggtgggcgcctgtagtcccagctgcttgggaggctgaggcaggagaatagcgtgaacctgggagttggagcttgcagtgagccgagatcgcaccactgcactctagcctgggcaacagagcaagactccatctacaaaaaaaaaaaaaagaaatgaagtcttgatacggtggctcatgcctgtaatcccagcactttgggaggccaaggcaggcggatcacgagctcaggagatcgagaccatcctggccaacgtggcgaaacccagtctctactaaagatacaaaaaattagccaggcatggtggcggg

SEQ ID NO: 4 - RNA, 5175 nt, Homo sapiens
gccccgagugcaaucgcgggaagccaggguuuccagcuaggacacagcaggucgugauccgggucgggacacugccuggcagaggcugcgagcauggggcccuggggcuggaaauugcgcuggaccgucgccuugcuccucgccgcggcggggacugcagugggcgacagaugygaaagaaacgaguuccagugccaagacgggaaaugcaucuccuacaagugggucugcgauggcagcgcugagugccaggauggcucugaugagucccaggagacgugcuugucugucaccugcaaauccggggacuucagcugugggggccgugucaaccgcugcauuccucaguucuggaggugcgauggccaaguggacugcgacaacggcucagacgagcaaggcuguccccccaagacgugcucccaggacgaguuucgcugccacgaugggaagugcaucucucggcaguucgucugugacucagaccgggacugcuuggacggcucagacgaggccuccugcccggugcucaccuguggucccgccagcuuccagugcaacagcuccaccugcaucccccagcugugggccugcgacaacgaccccgacugcgaagauggcucggaugaguggccgcagcgcuguaggggucuuuacguguuccaaggggacaguagccccugcucggccuucgaguuccacugccuaaguggcgagugcauccacuccagcuggcgcugugaugguggccccgacugcaaggacaaaucugacgaggaaaacugcgcuguggccaccugucgcccugacgaauuccagugcucugauggaaacugcauccauggcagccggcagugugaccgggaauaugacugcaaggacaugagcgaugaaguuggcugcguuaaugugacacucugcgagggacccaacaaguucaagugucacagcggcgaaugcaucacccuggacaaagucugcaacauggcuagagacugccgggacuggucagaugaacccaucaaagagugcgggaccaacgaaugcuuggacaacaacggcggcuguucccacgucugcaaugaccuuaagaucggcuacgagugccugugccccgacggcuuccagcugguggcccagcgaagaugcgaagauaucgaugagugucaggaucccgacaccugcagccagcucugcgugaaccuggaggguggcuacaagugccagugugaggaaggcuuccagcuggacccccacacgaagrccugcaaggcugugggcuccaucgccuaccucuucuucaccaaccggcacgaggucaggaagaugacgcuggaccggagcgaguacaccagccucauccccaaccugaggaacguggucgcucuggacacggagguggccagcaauagaaucuacuggucugaccugucccagagaaugaucugcagcacccagcuugacagagcccacggcgucucuuccuaugacaccgucaucagcagrgacauccaggcccccgacgggcuggcuguggacuggauccacagcaacaucuacuggaccgacucuguccugggcacugucucuguugcggauaccaagggcgugaagaggaaaacguuauucagggagaacggcuccaagccaagggccaucgugguggauccuguucauggcuucauguacuggacugacuggggaacuccygccaagaucaagaaagggggccugaaugguguggacaucuacucgcuggugacugaaaacauucaguggcccaauggcaucacccuagaucuccucaguggccgccuyuacuggguugacuccaaacuucacuccaucucaagcaucgaugucaaygggggcaaccggaagaccaucuuggaggaugaaaagaggcuggcccaccccuucuccuuggccgucuuugaggacaaaguauuuuggacagauaucaucaacgaagccauuuucagugccaaccgccucacagguuccgaugucaacuuguuggcugaaaaccuacuguccccagaggauaugguycucuuccacaaccucacccagccaagaggagugaacuggugugagaggaccacccugagcaauggcggcugccaguaucugugccucccugccccgcagaucaacccccacucgcccaaguuuaccugcgccugcccggacggcaugcugcuggccagggacaugaggagcugccucacagaggcugaggcugcaguggccacccaggagacauccaccgucaggcuaaaggucagcuccacagccguaaggacacagcacacaaccacccgrccuguucccgacaccucccggcugccuggggccaccccugggcucaccacgguggagauagugacaaugucucaccaagcucugggcgacguugcuggcagaggaaaugagaagaagcccaguagcgugagggcucuguccauuguccuccccaucgugcuccucgucuuccuuugccugggggucuuccuucuauggaagaacuggcggcuuaagaacaucaacagcaucaacuuugacaaccccgucuaucagaagaccacagaggaugagguccacauuugccacaaccaggacggcuacagcuaccccucgagacagauggucagucuggaggaugacguggcgugaacaucugccuggagucccgccccugcccagaacccuuccugagaccucgccrgccuuguuuuauucaaagacagagaagaccaaagcauugccugccagagcuuuguuuuauauauuuauucaucugggaggcagaacaggcuucggacagugcccaugcaauggcuuggguugggauuuugguuucuuccuuuccugugaaggauaagagaaacaggcccggggggaccaggaugacaccuccauuucucuccaggaaguuuugaguuucucuccaccgugacacaauccucaaacauggaagaugaaagggsaggggaugucaggcccagagaagcaaguggcuuucaacacacaacagcagauggcaccaacgggacccccuggcccugccucauccaccaaucucuaagccaaaccccuaaacucaggagucaacguguuuaccucuucuaugcaagccuugcuagacagccagguuagccuuugcccugucacccccraaucaugacccacccagugucuuucgagguggguuuguaccuuccuuaagccaggaaagggauucauggcgucggaaaugaucuggcugaauccgugguggcaccgagaccaaacucauucaccaaaugaugccacuucccagaggcagagccugagucacyggucacccuuaauauuuauuaagugccugagacacccgguuaccuuggccgugaggacacguggccugcacccagguguggcugucaggacaccagccuggugcccruccucccgaccccuacccacuuccauucccguggucuccuugcacuuucucaguucagaguuguacacuguguacauuuggcauuuguguuauuauuuugcacuguuuucugucguguguguugggaugggaucccaggccagggaaagcccgugucaaugaaugccggggacagagaggggcagguugaccgggacuucaaagccgugaucgugaauaucgagaacugccauugucgucuuuauguccgcccaccuagugcuuccacuucuaugcaaaugccuccaagccauucacuuccccaaucuugucguugauggguauguguuuaaaacaugcacggugaggccgggcgcaguggccucacgccuguaaucccagcacuuugggaggccgaggcggguggaucaugaggucaggagaucgagaccauccuggcuaacamggugaaaccccgucucuacuaaaaauacaaaaaauuagccgggcgygguggygggcaccuguagucccagcuacucgggaggcugaggcaggagaauggugugaacccgggaagcggagcuugcagugagccgagauugcgccacugcaguccgcagucuggccugggcgacagagcgagacuccgucucaaaaaaaacaaaacaaaaaaaaaccaugcauggugcaucagcagcccauggccucuggccaggcauggcgaggcugaggugggaggaugguuugagcucaggcauuugaggcugucgugagcuaugauuaugccacugcuuuccagccugggcaacauaguaagaccccaucucuuaaaaaaugaauuuggccagacacaggugccucacgccuguaaucccagcacuuugggaggcugagcuggaucacuugaguucaggaguuggagaccaggccugagcaacaaagcgagaucccaucucuacaaaaaccaaaaaguuaaaaaucagcuggguaygguggcacgugccugugaucccagcuacuugggaggcugaggcaggaggaucgccugagcccaggagguggagguugcagugagccaugaucgagccacugcacuccagccugggcaacagaugaagacccuauuucagaaauacaacuauaaaaaaaauaaauaaauccuccagucuggaucguuugacgggacuucagguucuuucugaaaucgccguguuacuguugcacugauguccggagagacagugacagccuccgucagacucccgcgugaagaugucacaagggauuggcaauuguccccagggacaaaacacugugucccccccagugcagggaaccgugauaagccuuucugguuucggagcacguaaaugcgucccuguacagauaguggggauuuuuuguuauguuugcacuuuguauauugguugaaacuguuaucacuuauauauauauauacacacauauauauaaaaucuauuuauuuuugcaaacccugguugcuguauuuguucagugacuauucucggggcccuguguaggggguuauugccucugaaaugccucuucuuuauguacaaagauuauuugcacgaacuggacugugugcaacgcuuuuugggagaaugauguccccguuguauguaugaguggcuucugggagaugggugucacuuuuuaaaccacuguauagaagguuuuuguagccugaaugucuuacugugaucaauuaaauuucuuaaaug

SEQ ID NO: 5 - DNA, 121 bp, Homo sapiens
tctgattctggcgttgagagaccctttctccttttcctctctctcagtgggcgacagatgygaaagaaacgagttccagtgccaagacgggaaatgcatctcctacaagtgggtctgcgat

SEQ ID NO: 6 - DNA, 121 bp, Homo sapiens
gtgctgtgagtcccctttgggcatgatatgcatttatttttgtaatagagacagggtctcrccatgttggccaggctggtcttgaatttctggtctcaagtgatccgctggcctcggcctc

SEQ ID NO: 7 - DNA, 121 bp, Homo sapiens
agtgcctgtgccccgacggcttccagctggtggcccagcgaagatgcgaaggtgatttccsggtgggactgagccctgggccccctctgcgcttcctgacatggcaaccaaacccctcatg

SEQ ID NO: 8 - DNA, 121 bp, Homo sapiens
ctggagggtggctacaagtgccagtgtgaggaaggcttccagctggacccccacacgaagrcctgcaaggctgtgggtgagcacgggaaggcggcgggtgggggcggcctcaccccttgca

SEQ ID NO: 9 - DNA, 121 bp, Homo sapiens
tgcaggtgagcgtcgcccctgcctgcagccttggcccgcaggtgagatgagggctcctggygctgatgcccttctctcctcctgcctcagcacccagcttgacagagcccacggcgtctct

SEQ ID NO: 10 - protein, 400 aa, Homo sapiens
  VARIANT (78)..(78): Xaa is Ala or Thr
GTNECLDNNGGCSHVCNDLKIGYECLCPDGFQLVAQRRCEDIDECQDPDTCSQLCVNLEGGYKCQCEEGFQLDPHTKXCKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLT

SEQ ID NO: 11 - protein, 749 aa, Homo sapiens
  VARIANT (370)..(370): Xaa is Ala or Thr
AVGDRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKXCKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQ

SEQ ID NO: 12 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYNNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPETDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVVENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDVLEHVKHFVINLIGDFEVAEKINAFRAKVHELIERYEVDQQIQVLMDKLVELTHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMQNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPHFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVAKTTKAEIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFITPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTLAHRDFSAEYEEDGKFEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQKFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFNDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 13 - protein, 317 aa, Homo sapiens [sequence omitted]

SEQ ID NO: 14 - DNA, 121 bp, Homo sapiens
cctcagcacccagcttgacagagcccacggcgtctcttcctatgacaccgtcatcagcagrgacatccaggcccccgacgggctggctgtggactggatccacagcaacatctactggacc

SEQ ID NO: 15 - DNA, 121 bp, Homo sapiens
cctcacagctattctctgtcctcccaccagcttcatgtactggactgactggggaactccygccaagatcaagaaagggggcctgaatggtgtggacatctactcgctggtgactgaaaac

SEQ ID NO: 16 - DNA, 121 bp, Homo sapiens
cctaggtatgttcgcaggacagccgtcccagccagggccgggcacaggctggaggacagaygggggttgccaggtggctctgggacaagcccaagctgctccctgaaggtttccctctttc

SEQ ID NO: 17 - DNA, 121 bp, Homo sapiens
ctctccaggtgcttttctgctaggtccctggcagggggtcttcctgcccggagcagcgtgkccaggccctcaggaccctctgggactggcatcagcacgtgacctctccttatccacttgt

SEQ ID NO: 18 - DNA, 121 bp, Homo sapiens
ttctgctaggtccctggcagggggtcttcctgcccggagcagcgtggccaggccctcaggmccctctgggactggcatcagcacgtgacctctccttatccacttgtgtgtctagatctcc

SEQ ID NO: 19 - DNA, 121 bp, Homo sapiens
gcatcagcacgtgacctctccttatccacttgtgtgtctagatctcctcagtggccgcctytactgggttgactccaaacttcactccatctcaagcatcgatgtcaacgggggcaaccgg

SEQ ID NO: 20 - protein, 860 aa, Homo sapiens
  VARIANT (391)..(391): Xaa is Ala or Thr
MGPWGWKLRWTVALLLAAAGTAVGDRCERNEFQCQDGKCISYKWVCDGSAECQDGSDESQETCLSVTCKSGDFSCGGRVNRCIPQFWRCDGQVDCDNGSDEQGCPPKTCSQDEFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKXCKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIVTMSHQALGDVAGRGNEKKPSSVRALSIVLPIVLLVFLCLGVFLLWKNWRLKNINSINFDNPVYQKTTEDEVHICHNQDGYSYPSRQMVSLEDDVA

SEQ ID NO: 21 - DNA, 20 bp, Artificial Sequence
  Amplicon 2 Primer A
attccctgggaatcagactg

SEQ ID NO: 22 - DNA, 20 bp, Artificial Sequence
  Amplicon 2 Primer B
taagaatcgtgtcacaggcc

SEQ ID NO: 23 - DNA, 20 bp, Artificial Sequence
  Amplicon 7 Primer A
ggcaggagaatcacttgaac

SEQ ID NO: 24 - DNA, 20 bp, Artificial Sequence
  Amplicon 7 Primer B
ttccatgcaggtggaatctc

SEQ ID NO: 25 - DNA, 20 bp, Artificial Sequence
  Amplicon 8 Primer A
attacatctcccgagaggct

SEQ ID NO: 26 - DNA, 20 bp, Artificial Sequence
  Amplicon 8 Primer B
gttcagaggatgaaactccc

SEQ ID NO: 27 - DNA, 18 bp, Artificial Sequence
  Amplicon 23 Primer A
ggggaggcactcttggtt

SEQ ID NO: 28 - DNA, 18 bp, Artificial Sequence
  Amplicon 23 Primer B
gctccctccattccctct

SEQ ID NO: 29 - DNA, 20 bp, Artificial Sequence
  Amplicon 10 Primer A
gcaggactatttcccaagcc

SEQ ID NO: 30 - DNA, 20 bp, Artificial Sequence
  Amplicon 10 Primer B
tgagctacgattgcgccagt

SEQ ID NO: 31 - DNA, 20 bp, Artificial Sequence
  Amplicon 11 Primer A
ttcaggctcacatgtggttg

SEQ ID NO: 32 - DNA, 20 bp, Artificial Sequence
  Amplicon 11 Primer B
gcgttcatcttggcttgagt

SEQ ID NO: 33 - DNA, 20 bp, Artificial Sequence
  Amplicon 12 Primer A
tactccagcctgggcaacaa

SEQ ID NO: 34 - DNA, 20 bp, Artificial Sequence
  Amplicon 12 Primer B
cccgactcatgagtccttac

SEQ ID NO: 35 - DNA, 20 bp, Artificial Sequence
  Amplicon 13 Primer A
catgttgaccaggctagtct

SEQ ID NO: 36 - DNA, 20 bp, Artificial Sequence
  Amplicon 13 Primer B
gactccatctcgtgaccaaa

SEQ ID NO: 37 - DNA, 20 bp, Artificial Sequence
  Amplicon 15 Primer A
gaccaggagtcaaggttatg

SEQ ID NO: 38 - DNA, 19 bp, Artificial Sequence
  Amplicon 15 Primer B
cattcacctaatgctgtcc

SEQ ID NO: 39 - DNA, 20 bp, Artificial Sequence
  Amplicon 16 Primer A
tgaatccggtactcaccgtc

SEQ ID NO: 40 - DNA, 20 bp, Artificial Sequence
  Amplicon 16 Primer B
agccagatcatttccgacgc

SEQ ID NO: 41 - DNA, 20 bp, Artificial Sequence
  Amplicon 17 Primer A
gagtttctctccaccgtgac

SEQ ID NO: 42 - DNA, 20 bp, Artificial Sequence
  Amplicon 17 Primer B
gacgacaatggcagttctcg

SEQ ID NO: 43 - DNA, 20 bp, Artificial Sequence
  Amplicon 18 Primer A
ccgtggtctccttgcacttt

SEQ ID NO: 44 - DNA, 20 bp, Artificial Sequence
  Amplicon 18 Primer B
tgcctgagctcaaaccatcc

SEQ ID NO: 45 - DNA, 121 bp, Homo sapiens
cagtggccgcctctactgggttgactccaaacttcactccatctcaagcatcgatgtcaaygggggcaaccggaagaccatcttggaggatgaaaagaggctggcccaccccttctccttg

SEQ ID NO: 46 - DNA, 121 bp, Homo sapiens
cctcacaggttccgatgtcaacttgttggctgaaaacctactgtccccagaggatatggtyctcttccacaacctcacccagccaagaggtaagggtgggtcagccccacccccccaacct

SEQ ID NO: 47 - DNA, 121 bp, Homo sapiens
atccaccgtcaggctaaaggtcagctccacagccgtaaggacacagcacacaaccacccgrcctgttcccgacacctcccggctgcctggggccacccctgggctcaccacggtggagata

SEQ ID NO: 48 - DNA, 121 bp, Homo sapiens
ttaaaccggaattgagtcctacaacctcgataactcacaaataagcccgtgtggcctctcrcagacttgggaagttctccaagtgtccagggagatgtgccaggcgctttcctgccgtgac

SEQ ID NO: 49 - DNA, 121 bp, Homo sapiens
ttgtcctccccatcggtaagcgcgggccggtcccccagcgtcccccaggtcacagcctccygctatgtgacctcgtgcctggctggttgggcctgttcactttttctcctggacagggaac

SEQ ID NO: 50 - DNA, 121 bp, Homo sapiens
tctggcggctcctgggggaacatgcttggggatcaggctgggggaggctgccaggcccagraggtgagaagtaggtggcctccagccgtgtttcctgaatgctggactgatagtttccgct

SEQ ID NO: 51 - DNA, 121 bp, Homo sapiens
tgggggaggctgccaggcccaggaggtgagaagtaggtggcctccagccgtgtttcctgartgctggactgatagtttccgctgtttaccatttgttggcagagacagatggtcagtctgg

SEQ ID NO: 52 - DNA, 121 bp, Homo sapiens
gtggcgtgaacatctgcctggagtcccgtccctgcccagaacccttcctgagacctcgccrgccttgttttattcaaagacagagaagaccaaagcattgcctgccagagctttgttttat

SEQ ID NO: 53 - DNA, 121 bp, Homo sapiens
caggaagttttgagtttctctccaccgtgacacaatcctcaaacatggaagatgaaagggsaggggatgtcaggcccagagaagcaagtggctttcaacacacaacagcagatggcaccaa

SEQ ID NO: 54 - DNA, 121 bp, Homo sapiens
tttacctcttctatgcaagccttgctagacagccaggttagcctttgccctgtcacccccraatcatgacccacccagtgtctttcgaggtgggtttgtaccttccttaagccaggaaagg

SEQ ID NO: 55 - DNA, 121 bp, Homo sapiens
caccgagaccaaactcattcaccaaatgatgccacttcccagaggcagagcctgagtcacyggtcacccttaatatttattaagtgcctgagacacccggttaccttggccgtgaggacac

SEQ ID NO: 56 - DNA, 121 bp, Homo sapiens
ggccgtgaggacacgtggcctgcacccaggtgtggctgtcaggacaccagcctggtgcccrtcctcccgacccctacccacttccattcccgtggtctccttgcactttctcagttcagag

SEQ ID NO: 57 - DNA, 121 bp, Homo sapiens
tttgggaggccgaggcgggtggatcatgaggtcaggagatcgagaccatcctggctaacamgtgaaaccccgtctctactaaaaatacaaaaaattagccgggcgtggtggcgggcacctg

SEQ ID NO: 58 - DNA, 120 bp, Homo sapiens
tgggaggccgaggcgggtggatcatgaggtcaggagatcgagaccatcctggctaacacggaaaccccgtctctactaaaaatacaaaaaattagccgggcgtggtggcgggcacctgta

SEQ ID NO: 59 - DNA, 120 bp, Homo sapiens
gtggatcatgaggtcaggagatcgagaccatcctggctaacacgtgaaaccccgtctctaaaaatacaaaaaattagccgggcgtggtggcgggcacctgtagtcccagctactcgggag

SEQ ID NO: 60 - DNA, 121 bp, Homo sapiens
ccatcctggctaacacgtgaaaccccgtctctactaaaaatacaaaaaattagccgggcgyggtggcgggcacctgtagtcccagctactcgggaggctgaggcaggagaatggtgtgaac

SEQ ID NO: 61 - DNA, 121 bp, Homo sapiens
tggctaacacgtgaaaccccgtctctactaaaaatacaaaaaattagccgggcgtggtggygggcacctgtagtcccagctactcgggaggctgaggcaggagaatggtgtgaacccggga

SEQ ID NO: 62 - DNA, 119 bp, Homo sapiens
tgggaggccgaggcgggtggatcatgaggtcaggagatcgagaccatcctgctaacacggaaaccccgtctctactaaaaatacaaaaaattagccgggcgtggtggcgggcacctgta

SEQ ID NO: 63 - DNA, 123 bp, Homo sapiens
gtggatcatgaggtcaggagatcgagaccatcctggctaacacgtgaaaccccgtctctactaaaaatacaaaaaattagccgggcgtggtggcgggcacctgtagtcccagctactcgggag